(19) United States
(12) Patent Application Publication
(10) Pub. No.: US 2025/0170097 A1
(43) Pub. Date: May 29, 2025

(54) CATHEPSIN INHIBITORS AND USE THEREOF IN A METHOD FOR DETECTING AND TREATING RESISTANCE TO IMMUNOTHERAPY

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL); The Cleveland Clinic Foundation, Cleveland, OH (US)

(72) Inventors: Galia BLUM, Jerusalem (IL); Brian GASTMAN, Cleveland, OH (US); Ihab ABD-ELRAHMAN, Abu Gosh (IL); Noha KHAIRI, Jerusalem (IL); Dina BEN YEHUDA, Mevaseret Zion (IL); Miri GUTMAN, Jerusalem (IL); Riki PERLMAN, Beit-Zayit (IL); Reut SINAI-TURYANSKY, Jerusalem (IL)

(73) Assignees: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL); The Cleveland Clinic Foundation, Cleveland, OH (US)

(21) Appl. No.: 19/034,106

(22) Filed: Jan. 22, 2025

Related U.S. Application Data
(63) Continuation of application No. PCT/IL2023/050766, filed on Jul. 23, 2023.
(60) Provisional application No. 63/369,131, filed on Jul. 22, 2022.
(60) Provisional application No. 63/369,689, filed on Jul. 28, 2022.

(51) Int. Cl. A61K 31/4015; A61K 47/68; A61P 35/02; C07D 207/448; C07K 16/40

(52) U.S. Cl. CPC A61K 31/4015; A61K 47/6871; A61P 35/02; C07D 207/448; C07K 16/40

(57) ABSTRACT

The present invention is directed to cathepsin inhibitor conjugates, precursors and compositions comprising thereof. Further, methods of use such as for the treatment and prevention of a disorder associated with abnormal cathepsin activity in a subject in need thereof are also provided.

### CROSS REFERENCE TO RELATED APPLICATIONS

This application is a Bypass Continuation of PCT Patent Application No. PCT/IL2023/050766 having International filing date of Jul. 23, 2023, which claims the benefit of priority to U.S. Provisional Patent Application No. 63/369,131 filed on Jul. 22, 2022, entitled “METHOD FOR DETECTING AND TREATING RESISTANCE TO IMMUNOTHERAPY”, and U.S. Provisional Patent Application No. 63/369,689 filed on Jul. 28, 2022, entitled “TREATING DISEASES BY CATHEPSIN INHIBITORS” all of which are hereby incorporated by reference in their entirety.

### FIELD OF THE INVENTION

The present invention relates to novel compounds for the treatment of medical disorders associated with elevated cathepsin activity. Further, the present invention relates to the use of a cathepsin inhibitor-based probe in a method for determining suitability of a subject being a candidate for anti-cancer immunotherapy.

### BACKGROUND OF THE INVENTION

Macrophages constitute a prominent set of immune cells that are phagocytic in nature and are present in almost all tissues. They undergo a polarization process where they express different surface markers and functional programs in response to microenvironment stimuli such as cytokines and other signaling mediators. Classically activated macrophages (M1) produce pro-inflammatory cytokines and reactive oxygen/nitrogen species, which are crucial for host defenses and tumor cell killing. Alternatively activated macrophages (M2) produce anti-inflammatory cytokines and are involved in the resolution of inflammation. Like other suppressor/regulatory immune cells, they not only suppress the destructive immunity against tumor cells, but also promote angiogenesis and matrix remodeling, making the tumor microenvironment conducive to cancer progression and metastasis. There is now strong interest in preventing the various immune suppressor populations to improve cancer outcomes, including M2 macrophages. Several reports show that combining strategies to inhibit M2 cells and checkpoint inhibition are highly synergistic. More recent data indicate that M2 cells have a direct role in anti-PD-1 based therapy resistance in melanoma. So, although anti-PD-1/PDL-1 strategies can convert some M2 cells to M1, there is still a need to overcome the full breadth of M2 activity to enhance immunotherapy effectiveness.

The M2 macrophages promotion of tumor progression is greatly reliant on high activity of cathepsin proteases that facilitate M2's roles in tumor progression including angiogenesis, degradation of extracellular matrix, vascular basement membrane and activation of angiogenic growth factors. In accordance with their central role in macrophage function, we have shown that inhibiting cathepsin activity leads to dramatic tumor shrinkage and specific Tumor Associated Macrophage (TAM) cell death.

Probes that report on protease activity, specifically cathepsin activity, have been widely developed over the last decades for both research and diagnostic uses. Cathepsin activity-based probes (ABPs) covalently bind the protease as a result of the protease activity. The covalent nature of ABPs leads to their accumulation in cells, when administered intravenously. Cathepsin ABPs highly accumulate specifically in TAMs because of their high cathepsin activity and phagocytic potency. Our published gold nanoparticulate (GNP) cathepsin ABPs highly accumulate in vivo in tumors enabling detection of small tumors by X-ray computer tomography (CT) and the cathepsin activity within the tumors (see Tsvirkun, D., et al. (2018), “CT Imaging of Enzymatic Activity in Cancer Using Covalent Probes Reveal a Size-Dependent Pattern.” J Am Chem Soc 140 (38): 12010-12020, herein incorporated by reference in its entirety).

There is a very great need to quickly detect immunotherapy resistant patients and thereby enable alternative therapies.

The recognition that M2 macrophages are a major target to improve immunotherapy is now a well-accepted fact. Most groups are focusing on a receptor, Colony-stimulating factor 1 (CSF1) a key regulator of monocyte/macrophage differentiation that sustains the pro-tumorigenic functions of TAMs/M2 cells. Monotherapy and combinations of immunotherapy with antibodies targeting the CSF-1 receptor, like AMGEN's AMG 820 or Five Prime Therapeutics' drug FPA008/cabiralizumab (see clinicaltrials.gov-NCT02526017 and NCT03502330 for example) are completed or in progress. Antibody drugs have many disadvantages, such as their production cost, stability, and immunogenicity. Even in the case of anti-PD-1 agents these factors have led to the development of small-molecule inhibitors of PD-1 and its ligand PD-L1. European Patent EP3884948 refers to a cathepsin inhibitor, especially cathepsin S inhibitor administered together with anti-cancer immunotherapy for use in the prevention and/or treatment of a cancer.

Thus, there is an unmet need for reliable methods suitable for detecting IT resistant patients to enable substitute therapies. This disclosure addresses this as well as other needs.

### SUMMARY OF THE INVENTION

The present disclosure provides a conjugate comprising a cysteine cathepsin inhibitor covalently bound to a targeting group (hereinafter “T”). In some embodiments, “T” comprises a macromolecule. In some embodiments, “T” is capable of binding an extracellular domain within a target cell. In some embodiments, the extracellular domain is capable of inducing cellular internalization of the conjugate upon binding thereto. Uses of these conjugates in the treatment of medical disorders, for example cancer and viral infections, are also provided.

The present invention further provides methods for determining whether a subject, being a candidate for anti-cancer immunotherapy, has a high likelihood of being non-responsive (refractory) to the treatment the method comprising determining in the subject or in a sample obtained from the subject the level of cathepsin expression product or the level of cathepsin activity, wherein a level higher than a predetermined threshold level indicates the subject has a high likelihood of being non-responsive to the anti-cancer immunotherapy.

In one aspect of the invention, there is provided A conjugate including any salt thereof and any enantiomer thereof, wherein said conjugate is represented by Formula I:

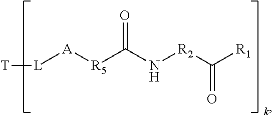

wherein R1 comprises any one of: chloromethyl ketone, acyloxymethyl ketone, a Michael acceptor, phosphonate, cyano group, or

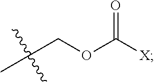

wherein X is selected from a substituted or unsubstituted alkyl; a substituted or unsubstituted alkenyl, a substituted or unsubstituted aryl, a substituted or unsubstituted heteroaryl; R2 comprises a substituted or unsubstituted alkylamine, or —[C(D′)₂]ₙ—, wherein each D′ is independently H, a substituent, or an optionally substituted C1-C20 alkylamine; and wherein at least one D′ is the optionally substituted C1-C20 alkylamine;
R5 comprises

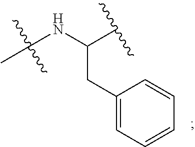 [SMILES: *NC(*)Cc1ccccc1]

A represents at least one amino acid residue; n is an integer ranging between 1 and 3; k is an integer ranging between 1 and 30; L represents a spacer; and T comprises a small molecule comprising a thiol or an amino and having a binding affinity to an extracellular domain, or a macromolecule comprising a polyamino acid, a glycoprotein, or a polynucleic acid, including any salt, any conjugate or any combination thereof.

In another aspect, there is provided a compound represented by Formula VI:

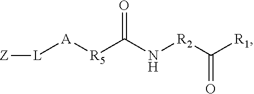

wherein R1 comprises any one of: chloromethyl ketone, acyloxymethyl ketone, a Michael acceptor, phosphonate, cyano group, or

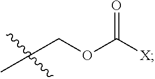

wherein X is selected from a substituted or unsubstituted alkyl; a substituted or unsubstituted alkenyl, a substituted or unsubstituted aryl, a substituted or unsubstituted heteroaryl; R2 comprises a substituted or unsubstituted alkylamine, or —[C(D′)₂]ₙ—; wherein each D′ is independently H, a substituent, or an optionally substituted C1-C20 alkylamine; and wherein at least one D′ is the optionally substituted C1-C20 alkylamine; R5 comprises

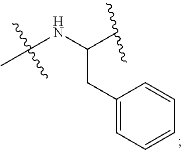 [SMILES: *NC(*)Cc1ccccc1]

A represents at least one amino acid residue; n is an integer ranging between 1 and 3; L represents a spacer; and Z is an electrophilic functionality having a reactivity to a thiol group, an amine group, or both.

In another aspect, there is provided a kit comprising the compound of the invention and optionally a macromolecule or a small molecule, wherein each of the macromolecule and the small molecule comprises a thio group, an amino group or both.

In another aspect, there is provided a pharmaceutical composition comprising the compound of the invention including any pharmaceutically acceptable salt thereof, and a pharmaceutically acceptable carrier.

In another aspect, there is provided a method for preventing or treating a disease or a disorder associated with a cathepsin activity in a subject in need thereof, comprising administering to said subject a therapeutically effective amount of the compound of the invention, or the pharmaceutical composition of the invention, thereby preventing or treating said disease or said disorder associated with said cathepsin activity in the subject.

In another aspect, there is provided a method of producing a conjugate, the method comprising: providing the compound of the invention and a reactant comprising a thiol group, an amino group or both; and contacting said compound with said reactant, thereby producing said conjugate. In one embodiment, contacting is under suitable conditions comprising a molar excess of the compound of the invention, optionally wherein the contacting step is performed in a suitable solvent (e.g. an aqueous solvent and/or a water-miscible organic solvent, or a water-immiscible organic solvent).

In another aspect, there is provided a method for determining whether a subject, being a candidate for anti-cancer immunotherapy treatment, is unlikely to respond to the treatment, the method comprising: determining in the subject or in a sample obtained from the subject the level of cathepsin activity; wherein a level higher than a predetermined threshold level indicates the subject unlikely to respond to the anti-cancer immunotherapy; thereby determining whether a subject is unlikely to respond to anti-cancer immunotherapy treatment.

In one embodiment, cathepsin-activity based probe is represented by Formula 1:

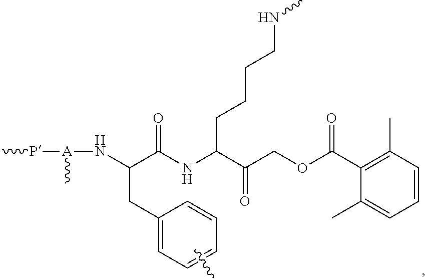

wherein: P′ is an amine protecting group; A is a bond or an amino acid residue; a wavy bond is absent or represents an attachment point to H, or to an imaging moiety, wherein at least one wavy bond is the attachment point to the imaging moiety. said at least one cathepsin inhibitor is the conjugate of the invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C4 are bar graphs and gel images representing the immune cell abundance and cathepsin activity in surgically removed human tissues from indicated metastatic melanoma patients. (1A) FACS analysis of the immune cell in the samples. Macrophage population and M2 macrophages found in tumor infiltrated lymphocyte (TIL) are significantly higher in refractory samples compared to Naïve samples (N=5). MΦ, *P=0.0484, and M2 MΦ, *P=0.0396. (1B) fluorescent SDS-PAGE of GB123 (Cy-5 linked GB111) labeled tissue lysates detecting the activity of cathepsins B, L and S, normal skin and inhibitor pretreatment (GB111-NH2) were used as controls. (1C1-1C4) quantification of the fluorescent SDS-PAGE bands of (1C1) cathepsins B, L and S; (1C2) cathepsin B; (1C3) cathepsins S; and (1C4) cathepsin L. *P value<0.05, **P value<0.005, ***P value<0.0005. Total cathepsin: Refractory VS GB111-NH2 ***P=0.0007, Naïve VS GB111-NH2 *P=0.0103, Naïve VS Refractory *P=0.0433, Skin VS Refractory **P=0.0043, Skin VS Naive *P=0.0346. Cathepsin B: Refractory VS GB111-NH2 **P=0.0023, Naïve VS GB111-NH2 **P=0.0047, Skin VS Refractory *P=0.0132, Skin VS Naive *P=0.0262. Cathepsin S: Refractory VS GB111-NH2 *P=0.0122, Naïve VS Refractory * P=0.0181. Cathepsin L: Refractory VS GB111-NH2 **P=0.0026, Naïve VS Refractory *** P=0.0004, Skin VS Refractory *P=0.0161.

FIGS. 2A-2B are bar graphs fluorescent microscopy images representing the cathepsin enzyme activity from patient tissue section. (2A) signal quantification, mean±SEM * P=0.0427. Scale bar=50 M; and (2B) fluorescent images of DAPI (blue), f GB123 (red) reflecting cathepsin activity, at 20× magnification.

FIGS. 3A-3D are bar graphs fluorescent microscopy images representing the cathepsin activity and co-localization with MΦ. (3A) The quantification of cathepsin enzyme activity detected by GB123 probe in patient samples. (MC is MΦ and CTS is cathepsin enzyme). **P=0.0031, ***P=0.005, *P=0.0242; (3B) fluorescence microscopy images detecting MΦ, CD68 (Green) cathepsin activity, GB123 (red), and DAPI (Blue), at a 20× magnification, scale bar=50 μM; (3C) Percentage of MΦ co-localized with active cathepsin labeled with GB123 in refractory and naïve samples detected in (c) *P=0.014; (3D) fluorescence microscopy images of refractory and naïve patient samples stained with CD68 (macrophages) GB123 (cathepsin activity), and DAPI (Blue), GB123 probe (red), CD68 (green), overlay of green and red (orange), at a 20× magnification, scale bar=50 μM.

FIGS. 4A1-4F3. are graphs and images representing the cathepsin activity in tumors after anti-PD-1 treatments in D4M or B16-F10 tumor bearing mice. (4A) the tumor size [cm³] over time in (4A1) control, (4A2) D4M and (4A3) B16-F10; (4B-4C) arc FACS analysis showing the percent of F4/80 and GB123 positive tumor macrophages by treatment in D4M and B16-F10 tumors, respectively; (4D1-4D2) SDS-PAGE of tumor lysates gels, band intensity reflects cathepsin activity in D4M and B16-F10 tumors lysates, respectively; (4E1-4E2) Quantification of the band intensity presented in 4D reflecting the (4E1) cathepsin B activity; (4E2) cathepsin S activity; and (4E3) cathepsin L activity, in D4M tumors lysates; (4F1-4F3) Quantification of the band intensity presented in 4D reflecting the (4F1) cathepsin B activity; (4F2) cathepsin S activity; and (4F3) cathepsin L activity, in D4M and B16-F10 tumors lysates. In FIGS. 4E and 4F the data presents the mean±SEM. P values lower than 0.05 were considered significant, *P<0.05, **P<0.01.

FIGS. 5A-5F. are bar graphs and fluorescence microscopy images representing microscopy analysis of D4M and B16-F10 tumor tissues of mice treated with IT compared to a control. (5A) fluorescence microscopy images detecting M, Cy3 (Green) cathepsin activity, GB123 (red), and DAPI (Blue) in D4M tumors; (5B) Quantifications of the microscopic images of D4M tumors, relative to cell number and the GB 123 signal intensity; (5C). Quantifications of the microscopic images of D4M tumors, relative to cell number and the Cy3 signal intensity, scale bar=50 μm; (5D). fluorescence microscopy images detecting MΦ, Cy3 (Green) cathepsin activity, GB123 (red), and DAPI (Blue) in B16-F10 tumors; (5E) Quantifications of the microscopic images of B16-F10 tumors, relative to cell number and the GB123 signal intensity, scale bar=50 μm; (5F). Quantifications of the microscopic images of B16-F10 tumors, relative to cell number and the Cy3 signal intensity. In FIGS. 5B, 5C, 5E, and 5F, the scale bar is 50 μm and the data is presented as mean±SEM. P values lower than 0.05 were considered significant, *P<0.05, **P<0.01.

FIGS. 6A-6B are graphs representing the effect a combined treatment has on B16-P10 tumors and the survival B16-F10 melanoma tumor bearing mice. (6A) B16-P10 tumor volume (cm³) over time; (6B) The percentage of mice that survived till day 18.

FIGS. 7A-B are images and bar graphs representing the optimal antibody drug ratio (ACE:MGB). (7A) SDS-PAGE of recombinant cathepsin L and B inhibition; and (7B) quantification of the gel bands.

FIGS. 8A1-8D2 are gel images and graphs presenting the inhibition of recombinant cathepsin B and recombinant cathepsin L by MGB (an exemplary compound of the invention) and GB111-NH2; (8A1-8A2) SDS-PAGE of recombinant cathepsin B, by MGB and GB111-NH2, respectively; (8B1-8B2) SDS-PAGE of recombinant cathepsin L, by MGB and GB111-NH2, respectively; (8C1-8C2) IC50 of recombinant cathepsin B and L inhibition, respectively, by MGB; and (8D1-8D2) IC50 of recombinant cathepsin B and L inhibition, respectively, by GB111-NH2.

FIGS. 9A1-9B2 are gel images and graphs presenting the inhibition of cathepsin L/B by MGB and GB111-NH2 in intact A549 cells. (9A1-9A2) SDS-PAGE of cathepsin, by MGB and GB111-NH2, respectively; and (9B1-9B2) IC50 of cathepsin inhibition by MGB and GB111-NH2, respectively.

FIGS. 10A-10B are graphs presenting the IC50 curves of cathepsin inhibition of (10A) MGB; and (10B) of an exemplary conjugate of the invention ACE2-ab-MGB comprising ACE-2 antibody covalently bound to MGB.

FIGS. 11A-11B are gel images presenting a comparison of the inhibition of cathepsins in three different cell lines, U87, HepG2, and A549. (11A) inhibition of cathepsins by ACE2-ab-MGB, as compared to un-bound ACE2-ab, MGB, and GB111-NH2; (11B) immunoprecipitation with anti-cathepsin L antibody.

FIGS. 12A-12D are bar graphs representing cell viability after treatment with ACE2-ab-MGB, MGB, or GB111-NH2 in the following cell lines (12A) A549; (12B) HepG2; (12C) Hacat; and (12D) U87.

FIGS. 13A-13C are gel images presenting B cathepsins activity in hematological malignancies patients' cells. (13A) B Cathepsins activity in hematological malignancies cell lines; (13B) Crude detergent lysates from cells that were pretreated with either cathepsin inhibitor GB111-NH2 or with control DMSO, bands of active cathepsins are marked with an arrow; (13C) Immunoprecipitation of specific cathepsins (CTS) L/S, detection using a probe for cathepsin activity. S-supernatant, not IP′ed, E-elution from beads (IP′ed).

FIGS. 14A-14D are bar graphs and images presenting the inhibition of cathepsins induces apoptosis in OCI-Ly19 cells. (14A) Caspase-3 activation; (14B) FACS analysis of stained cells with anti-Annexin V; (14C) Mononuclear cells from CLL patients were isolated by FICOL, treated with 5 uM GB111-NH₂ for 1 hour and stained with anti-Annexin V; and (14D) labeled crude detergent lysates.

FIGS. 15A-15D are bar graphs and images presenting (15A) LPS induced p65 (NF-κB subunit) activity in OCI-Ly19 cells and GB111-NH₂ negatively regulates NF-κB activity; (15B) gel image of the labeled crude lysate from (15A) FACS analysis of CD74 positive cells treated with GB111-NH2, LPS or both; (15C); and (15D) Western blot analysis of CD74 protein in DLBCL cell lines treated as indicated, loading control by GAPDH.

FIG. 16 is a bar graph presenting the percentage of cell apoptosis in OCI-Ly treated with GB111-NH2.

FIGS. 17A-17B are images presenting the activity of the linked-cathepsin inhibitor (MGB). (17A) Inhibition of purified recombinant cathepsins B and S by the control GB111-NH2 and IAE-GB (MNG); and (17B) inhibition of endogenous cathepsin activity within intact OCI-Ly19 cell.

FIG. 18A-18F are bar graphs and images presenting the relative quantification of cathepsin GB123 labeling intensity in lymphoma cells after different types of treatments (18A) in OCI-Ly3 cells treated with and without Rituximab; (18B) quantification of cathepsin labelled presented in 18A; (18C) FACS analysis of stained OCI-Ly3 after different types of treatment; (18D) crude detergent lysates; isolated mononuclear cells of (18E) chronic lymphocytic leukemia and (18F) marginal zone lymphoma taken from patients and treated with IAE-GB (MNG) or with a conjugated IAE-GB (R-IAE-GB).

### DETAILED DESCRIPTION OF THE INVENTION

The present invention, in some embodiments, provides a conjugate comprising a cysteine cathepsin inhibitor covalently bound to a targeting group T. Uses of these conjugates in the treatment of medical disorders, for example cancer and viral infections, and for improving immunotherapy are also provided. Methods for determining whether a subject, being a candidate for anti-cancer immunotherapy treatment, is unlikely to respond to the treatment are also provided.

The invention is based, at least in part, on the surprising finding of a new cathepsin inhibitor conjugate which effectively kills virally infected cells and can treat cancer and improve immunotherapy. The invention is further based on the surprising finding that M2 macrophage detection by GNP-ABP can be translated to the clinic to detect immunotherapy resistance. Specifically, the cathepsin activity-based probe, GB123, can be used as an agent to detect high cathepsin activity in TAMs and thereby the subset of patients with reduced response to immunotherapy. In mice, resistance to immunotherapy was detected by the presence of increased cathepsin activity, while lower and constant activity was detected in tumors sensitive to the treatment. Similarly, human resected melanoma's refractory to immunotherapy exhibited significantly higher cathepsin activity, which correlated with increased M2 Macrophages (MP). A combination of cathepsin inhibition and immunotherapy in mice models resulted in significantly smaller tumors demonstrating the effectiveness of the combination treatment. Further, the discovery of a new selective small molecule agent that targets cathepsin enzymes which are highly upregulated in M2/TAMs offers a major alternative to anti-CSF-1 based therapies without the negative aspects of an antibody.

### Conjugates

In one aspect of the invention, there is provided a conjugate obtained by a spontaneous reaction (e.g., a click reaction) between a compound and a targeting group; wherein the compound is represented by Formula A:

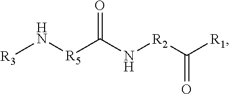

wherein: R1 is an electrophilic functionality selected to allow binding to a protease (or selected to be reactive towards a protease); R2 is a functionality selected from amine, ammonium, amino acid(s), or any functionality bearing an amine or an ammonium functionality; R3 is or comprises an electrophilic side chain, and R5 is a spacer moiety. In some embodiments, the targeting group has a reactivity towards R3. In some embodiments, R3 is capable of spontaneously reacting with a nucleophilic group of the targeting group. In some embodiments, the targeting group has a reactivity towards R3, wherein the reactivity is via the nucleophilic group. In some embodiments, the nucleophilic group is an amino acid side chain. In some embodiments, the nucleophilic group comprises a thio group, or an amino group. In some embodiments, the nucleophilic group is located on an amino acid side chain. In some embodiments, the nucleophilic group is a thio group of the cysteine side chain.

In some embodiments, the target associating group is encompassed by the definition of variable Z (e.g., of Formulae VI-VII), as disclosed hereinbelow.

In some embodiments, R2 is an amino acid having an amine sidechain (e.g., lysine, or a non-natural amino acid such as ornithine). In some embodiments, R2 is an amino acid residue, or a binding moiety selected to interact with a protease binding pocket. In some embodiments, in the compound of formula (A), the moiety —HN-R5-—C(═O)—NH—R2-C(═O)— constitutes an amino acid sequence comprising 3 to 10 amino acids. The compound may be thus designated R3-AA1---AA10-R1, wherein the expression “AA1---AA10” designates a three to ten amino acid sequence. In some embodiments, the amino acid sequence comprising 3 to 10 amino acids comprises at least one phenyl alanine and at least one lysine.

In some embodiments, R3 may be of a structure AA-L-R4, wherein AA is an amino acid or derivative or non-natural amino acid, R4 is a target associating group (i.e., a moiety obtained upon reaction of the compound with the targeting group, such as a click reaction product) and L is a spacer, as disclosed herein.

In some embodiments, a variable T of any one of Formulae I-V and B-C, as disclosed hereinbelow encompasses the targeting group (or a derivative thereof obtained upon reaction of the targeting group with the compound). In some embodiments, the targeting group comprises a small molecule, a macromolecule comprising a polyamino acid, a glycoprotein, or a polynucleic acid, including any salt, any conjugate, or any combination thereof. In some embodiments, the targeting group comprises a small molecule having a binding affinity to an extracellular domain and comprising a nucleophile capable of undergoing a spontaneous reaction with the target associating group of the compound. In some embodiments, the targeting group comprises a small molecule comprising at least one of a thio group, and an amino group.

The terms “extracellular domain” and “extracellular target” are used herein interchangeably.

In some embodiments, the conjugate of the invention is represented by Formula B:

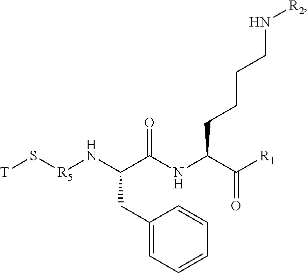

wherein each of R1 and R2 are as defined herein (e.g., R2 is alkyl, H or a fluorophore such as Cy-5), R5 is derived from an electrophilic functionality, T is the targeting group; S is optionally a sulfur atom of a cysteine amino acid present in the targeting group and wherein in a compound of formula (B) atom S is covalently associated to a carbon atom in group R5. In some embodiments, R5 is derived from an electrophilic functionality, wherein the term “derived” encompasses a reaction product of the target associating group and the targeting group). In some embodiments, R5 is a click reaction product. In some embodiments, R5 is derived from maleimide. In some embodiments, S—R5 is a succinimide-thioether. In some embodiments, R5 is

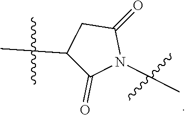 [SMILES: *C1CC(=O)N(*)C1=O]

In some embodiments, the conjugate of the invention is represented by Formula C:

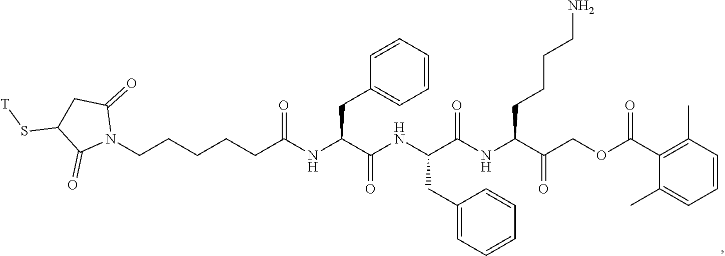

wherein S is a sulfur atom, e.g., of a cysteine present on T the targeting group. In some embodiments, T the targeting group is a functionality capable of associating to a molecule present on a membranal surface that enables entering of a cathepsin inhibitor of the invention to the target cell. In some embodiments, “T” is capable of associating to a cell type (hepatic cells, endothelial cells, neural cells), to a desired tissue even if the target tissue is composed of several types of cells (liver, lung, CNS) or to cells in a specific target cell state (cancer cells, ischemic cells, senescence cells etc.).

In some embodiments, T (the targeting group) is selected amongst antibodies, antigen binding fragment of an antibody, ligands to a membranal receptor or a receptor binding fragment of this ligand (the ligand being (that may be natural or synthetic, peptides, proteins, glycoproteins, hormones, drugs, small molecules), receptors, receptor fragments, or any thiol containing molecule, or nanoparticle.

In some embodiments, the term “targeting” refers to selective or preferential binding to a target entity on a membranal surface that enables entering of the active agent (here the cathepsin inhibitor) to the target cell. The binding may be preferential—i.e., not exclusive binding to the target but significantly higher binding to the target than to non-target. In some embodiments, the “binding” refers to a binding affinity and/or selectivity of the targeting group to the target. In some embodiments, the “selectivity” refers to a selective binding to the target, wherein selective encompasses at least 10 fold, at least 100 fold, at least 1000 fold greater selectivity constant to the target including any range between, as compared to a non-target (e.g., a mammalian cell devoid of the target entity, optionally a cell population which doesn't express the target entity). In some embodiments, the binding affinity of the targeting group is characterized by a dissociation constant (Kd) below 5 μM, below 1 μM, below 500 nM, below 100 nM, or between 10 nM and 1 μM, between 100 nM and 1 μM, including any range therebetween.

The target may be a cell of interest such as but not limited to: any cancerous cell, a hepatic cell, an endothelial cell, a neural cell, or a cell fragment. In some embodiments, the target is a tissue. In some embodiment a target tissue is composed of a variety of cell types.

The binding group may be any cysteine-containing molecule, or group of molecules capable of targeting as defined above and is typically an antibody, an antigen binding fragment of an antibody, a ligand to a membranal receptor or a receptor binding fragment of this ligand, a receptor, or a receptor fragment, or any thiol containing molecule, or nanoparticle.

In another aspect, there is provided a conjugate including any salt thereof and any stereoisomer thereof, wherein the conjugate is represented by Formula I:

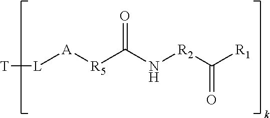

wherein R1:
R1 comprises any one of: chloromethyl ketone, acyloxymethyl ketone, a Michael acceptor, phosphonate, cyano group, or

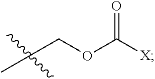

wherein X is selected from a substituted or unsubstituted alkyl; a substituted or unsubstituted alkenyl, a substituted or unsubstituted aryl, a substituted or unsubstituted heteroaryl; R2 comprises a substituted or unsubstituted alkylamine, or —[C(D′)₂]ₙ—, wherein each D′ is independently H, an amino acid side chain, a substituent, or an optionally substituted C1-C20 alkylamine; R5 comprises one or more

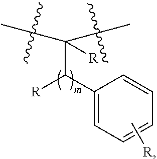

wherein each R is independently H, or represents at least one substituent; A represents at least one amino acid residue; m is an integer ranging between 1 and 5 (e.g., 1, 2, 3, 4, or 5 including any range between); n is an integer ranging between 1 and 3 (e.g., 1, 2 or 3 including any range between); k is an integer ranging between 1 and 30; L represents a spacer; and T comprises a small molecule having a binding affinity to an extracellular domain, or a macromolecule comprising a polyamino acid, a glycoprotein, or a polynucleic acid, including any salt, any conjugate or any combination thereof. In some embodiments, the small molecule has a reactivity to Z, wherein Z is as described herein. In some embodiments, the small molecule having a reactivity to Z comprises an amino group or a thiol group. In some embodiments, the small molecule has a reactivity to Z via an amino group or via a thiol group.

In some embodiments, R2 is —[C(D′)₂]ₙ—, wherein each D′ is independently H, a substituent, or an optionally substituted C1-C20 alkylamine; and wherein at least one D′ is the optionally substituted C1-C20 alkylamine, or the amino acid side chain. In some embodiments, R2 is C1-C20alkyl-NB′B′, wherein B′ is R′ or comprises a fluorophore, wherein R′ is as described herein. In some embodiments, R2 is C1-C20alkyl-NB′B′, wherein each B′ is H or at least one B′ is a fluorophore. In some embodiments, B′ comprises an imaging moiety. In some embodiments, B′ comprises a linker bound to the imaging moiety.

In some embodiments, the linker comprises an optionally substituted C1-C20 alkyl, a heteroatom (e.g., O, N, NH, or S), —C(O) NH—, —C(O)O—, —C(O)—, —C(O)S—, —C(NH) NH—, —C(NH)O—, —C(NH)S—, a C1-C10 aminoalkyl, a C1-C10 alkoxy, a C1-C10 mercaptoalkyl, —S—S—, —S—C(═O), including any combination thereof. In some embodiments, the linker comprises an oligomer (e.g., PEG, a peptide, oligosaccharide, a polyamine, etc.). In some embodiments, the linker is bound to N via an amide bond, and to the fluorophore via a C—C bond.

In some embodiments, the imaging moiety comprises a luminophore, a fluorophore, a CT contrast agent, an MRI contrast agent or a radiolabel, including any combination thereof.

In some embodiments, R5 is:

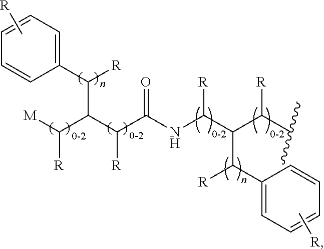

wherein M represents —O—, —S—, —NH—, or an attachment point to L.

In some embodiments, R5 is:

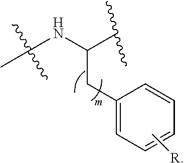

In some embodiments, the conjugate is represented by Formula I, wherein R5 is:

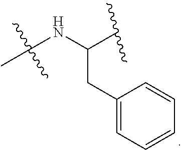 [SMILES: *NC(*)Cc1ccccc1]

In some embodiments, the conjugate is represented by Formula I, wherein R5 is:

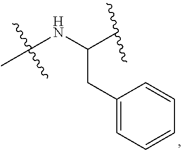 [SMILES: *NC(*)Cc1ccccc1]

and wherein R2 is C1-C20 alkylamine (e.g., C4-alkyl-NH2, C5-alkyl-NH2).

In some embodiments, k is an integer ranging between 1 and 30, between 2 and 30, between 2 and 20, between 2 and 15, between 2 and 10, between 4 and 20, between 3 and 20, between 2 and 8, between 5 and 20, including any range or value between. In some embodiments, k is between 2 and 20, or between 2 and 10. The conjugate of the invention (e.g. a conjugate material, as opposed to a single conjugate molecule) may comprise molecules each having the same or different value of k. In the case the conjugate material contains conjugate molecules with different values of k, the value of “k” as used herein refers to an average value for the entire composition. The average k value can be determined based on mass-spectrometry (e.g. MALDI).

The term “small molecule” encompasses any organic compound having a MW smaller than 1,000 Daltons (Da), or smaller than about 500 Da. The term “small molecule” further encompasses compounds having a binding affinity (e.g., characterized by a Kd below 1 uM) to a target, wherein the target is as disclosed herein (e.g., a surface molecule such as a cell membrane receptor, or a target cell). In some embodiments, the small molecule is a ligand of the surface molecule, wherein the ligand has a binding affinity and/or selectivity to the surface molecule, as disclosed herein. In some embodiments, the small molecule has a MW of 10-50 Da, 10-100 Da, 10-500 Da, 10-1,000 Da, 50-100 Da, 50-500 Da, 50-1,000 Da, 100-300 Da, 100-500 Da, 200-500 Da, 300-500 Da, 100-800 Da, 100-1,000 Da, 500-800 Da, 500-1,000 Da, 800-1,000 Da, or any range between.

The term “macro-molecule” encompasses any molecule having a MW greater than 10,000 Da. Macro-molecules may be a polymer (e.g., biopolymers, such as proteins or polyaminoacids, polynucleic acid, polysaccharides, including any co-polymer thereof).

In some embodiments, the macro molecule has a MW of 10,000-1,000,000 Da, 10,000-300,000 Da, 10,000-500,000 Da, 50,000-100,000 Da, 50,000-500,000 Da, or any range between. In some embodiments, the macro molecule has binding affinity and/or selectivity to a surface molecule, as disclosed herein. In some embodiments, the macromolecule is a natural or a synthetic polymer. In some embodiments, the macromolecule is a natural polymer consisting essentially of naturally occurring monomers (e.g., at least 90% or between 90 and 100% of the entire monomers in the polymer are naturally occurring monomers) such as amino acids, sugars, nucleic acids, carboxylic acids, aromatic or heteroaromatic molecules, etc. In some embodiments, the macromolecule is a synthetic polymer having a chemically modified monomer. In some embodiments, the chemically modified monomer comprises a moiety capable of reacting with Z group, such as by click reaction (e.g., azide, norbornene, tetrazine, maleimide, active ester, etc.). In some embodiments, the macromolecule is an antibody, or an antigen binding fragment of an antibody.

In some embodiments, A comprises between 1 and 3 amino acid residues. In some embodiments, A comprises one or two amino acid residues. In some embodiments, A comprises one or more amino acid residues, each amino acid residue is independently selected from an aromatic amino acid residue, alanine residue, glycine residue, or a branched amino acid residue. In some embodiments, A solely consists of one or more aromatic amino acid residue and/or alanine residue. In some embodiments, A consists of one or more aromatic amino acid residue(s) selected from Phe, Tyr, His and Trp, including any non-natural or modified amino acid residues.

In some embodiments, R1 is

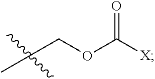

wherein X is selected from a substituted or unsubstituted alkyl; H, NH2, aminocarbonyl, amino, a substituted or unsubstituted alkenyl, a substituted or unsubstituted aryl, a substituted or unsubstituted heteroaryl, a substituted or unsubstituted carbocyclyl optionally comprising a heteroatom, wherein aryl, heteroaryl and carbocyclyl are optionally fused, polycyclic, or bi-cyclic rings. In some embodiments, R1 is

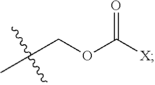

wherein X is a substituted aryl substituted aryl (including 2-nitro, 3-hydroxy benzyl and N-benzyloxycarbonyl [cbz]). In some embodiments, R1 is

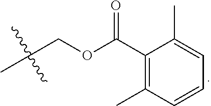 [SMILES: *COC(=O)c1c(C)cccc1C]

In some embodiments, the conjugate is represented by Formula I, wherein the moiety:

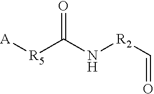

represents a peptide. In some embodiments, the peptide is between 2 and 10, between 2 and 5, between 2 and 7, between 2 and 4, or 2, 3, or 4 amino acid residues long.

In some embodiments, the moiety:

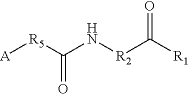

also referred to herein as “cathepsin-binding moiety”, is capable of binding cathepsin (e.g., via a covalent bond), and is further capable of inducing inhibition of cathepsin activity upon binding thereto. In some embodiments, cathepsin-binding moiety has cathepsin inhibitory activity, preferably cysteine cathepsin inhibition activity, more preferably cathepsin B, L and S inhibition activities. In some embodiments, the cathepsin-binding moiety is an irreversible cysteine cathepsin inhibitor. In some embodiments, the cathepsin-binding moiety is an irreversible cysteine cathepsin inhibitor; and is further characterized by selectivity to any of cathepsin B, L and S. The term “selectivity” is as disclosed hereinbelow.

In some embodiments, the conjugate is represented by Formula II:

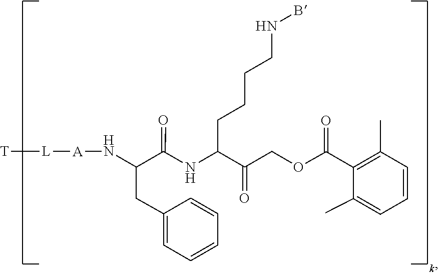

### or by Formula IIa:

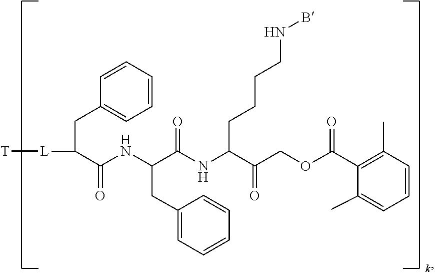

including any salt and any stereoisomer thereof; wherein T, L, A, B′ and k are as disclosed hereinabove.

In some embodiments, the conjugate is as disclosed herein (e.g., of Formulae I-IIa), wherein T and L are covalently bound to each other. In some embodiments, T and L are covalently bound to each other via a bond selected from amide bond, ester bond, S—S bond, and a click reaction product, including any combination thereof. In some embodiments, T and L are covalently bound to each other via a click reaction product. In some embodiments, the term “click reaction” refers to highly efficient and highly selective reaction with a reaction yield of almost 100%, along with negligible by products formation. The click reaction forms a covalent bond (conjugation) between two reactive groups attached to the same molecule or to different molecules. The reactive groups have a superior reactivity to each other and have only a negligible reactivity to any other functional group, thereby resulting in a highly specific/selective reaction. In some embodiments, the click reaction enables a specific conjugation of two different molecules (such as two polymers or a polymer and a small molecule).

In some embodiments, click reactions are well-known in the art and comprise inter alia Michael addition of maleimide and thiol (resulting in the formation of a succinimide-thioether); Michael addition of a Michael acceptor and a thiol; azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photo-click reaction, etc.

In some embodiments, T is bound to L via an amino group or via a thio group. In some embodiments, T is a macromolecule comprising a polyamino acid. In some embodiments, T comprises at least one Lys or Orn side chain. In some embodiments, T comprises at least one Cys side chain. In some embodiments, T comprises at least one reduced Cys side chain (i.e., having an —SH terminal group, as opposed to a disulfide bond formed between 2 Cys side chains). In some embodiments, T comprises k Cys side chains, wherein k is as described hereinabove. In some embodiments, T comprises k reduced Cys side chains. In some embodiments, T is bound to L via a sulfur atom of a cysteine side chain (e.g., via a sulfur atom of a reduced cysteine side chain).

In some embodiments, T is chemically modified and is bound to L via a moiety selected from a thio group, an amino group, 1,3-nitrone, azide, a diene, tetrazine, maleimide, an active ester, an alpha, beta-unsaturated keto acid derivative or any combination thereof, wherein the moiety as disclosed herein refers to T in its unconjugated form (i.e., in the original form before being conjugated to the moiety of the invention). A skilled artisan will appreciate, that in the conjugated form (i.e., within the conjugate of the invention) the thio group, amino group, alpha, beta-unsaturated keto acid derivative, etc. of the polymer generate a covalent bond with the moiety. Accordingly, the abovementioned groups of the polymer in the conjugated form undergo chemical modification, for example: amino group reacts with an active ester to form an amide bond, the thio group and maleimide and undergoes a Michael addition, so as to form a succinimide-thioether.

In some embodiments, succinimide-thioether is:

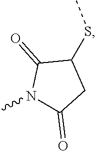

wherein (i) the wavy bond represents attachment point to the cathepsin-binding moiety (e.g., via a linker) and the dashed bond represents attachment point to T; or (ii) the wavy bond represents attachment point to T and the dashed bond represents attachment point to the cathepsin-binding moiety, and wherein S represents a sulfur atom of T.

In some embodiments, T is bound to L via a S—C bond. In some embodiments, T is bound to L via a succinimide-thioether.

In some embodiments, the conjugate is represented by Formula III:

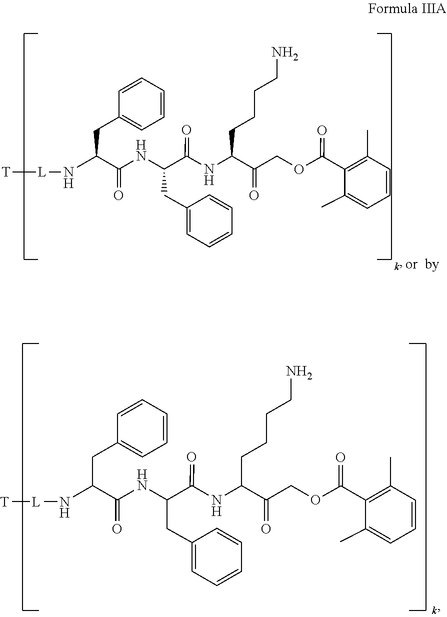

wherein T, L and k are as described hereinabove. In some embodiments, L comprises a small molecule such as a natural and/or unnatural amino acid or an oligomer comprising a natural and/or unnatural amino acid, a C5-C10cycloalkylene, optionally substituted C1-C6alkylene, —C(═O)—C1-C6alkylene, optionally substituted C6-C10arylene, a heteroaromatic ring; a bond (such as an amide bond, an ester bond, a thioester bond, a disulfide bond, —N—C(═O)—, —C(═O)N—, CONR′—, —CNNR′—, —CSNR′—, —NC(═O)O—, —NC(═S)O—, —NC(═S)N—, —SO2-, —SO—, —SR′, —C(═O)—, —OC(═O)—, —OC(═O)O—, —OC(═S)O—, and —OC(═S)N—; —S—C(═O), including any combination thereof), a click reaction product, or any combination thereof.

In some embodiments, L is or comprises a linear or a branched chain. In some embodiments, L comprises a backbone comprising a linear or a branched chain. In some embodiments, L comprises a cyclic backbone.

In some embodiments, the spacer has a MW less than 1,000 Da, less than 900 Da, less than 800 Da, less than 700 Da, less than 600 Da, less than 500 Da, less than 400 Da, less than 300 Da, less than 200 Da, less than 100 Da, or between 100 and 1000 Da.

In some embodiments, the spacer has an MW between 800 and 2,000 Da, between 800 and 1,000 Da, between 800 and 1,500 Da, between 800 and 900 Da, between 900 and 1,000 Da, between 1,000 and 1,100 Da, between 1000 and 1,200 Da, between 800 and 1,200 Da, between 1,000 and 3,000 Da, including any range between. In case where the composition of the invention comprises conjugates containing spacers characterized by different molecular weights, the term “MW” as used herein, refers to an average molecular weight of the spacers within the composition.

In some embodiments, the spacer is between 1 and 50, between 1 and 100, between 2 and 100, between 2 and 80, between 2 and 60, between 5 and 50, between 10 and 50, between 10 and 40, between 2 and 30, between 2 and 20, between 2 and 10, between 1 and 5, between 5 and 10, between 5 and 15, between 5 and 25, between 5 and 50 single C—C bonds long, including any range in between. In some embodiments, the spacer is characterized by an average molecular weight between 300 and 3,000 Da, and is between 2 and 50, between 2 and 10, between 2 and 20, between 5 and 30 C—C bonds long, including any range between.

In some embodiments, the spacer of the invention comprises a polymer or oligomer. In some embodiments, the spacer of the invention comprises a biocompatible and/or biodegradable polymer or oligomer.

In some embodiments, the biocompatible and/or biodegradable polymer or oligomer comprises a polyether (e.g., polyglycol ether), a polyester, a polyamide, or any combination or a co-polymer thereof. In some embodiments, the polyether is represented by a general formula: —(RO)x-, wherein R represents C1-C10 alkyl; and x is an integer ranging between 2 and 1000. In some embodiments, R represents an alkyl comprising between 1 and 10, between 1 and 2, between 2 and 4, between 4 and 10, between 2 and 5, between 1 and 5, between 5 and 10 carbon atoms, including any range between. In some embodiments, the polyether is PEG.

In some embodiments, the biocompatible and/or biodegradable polymer or oligomer is selected from the group consisting of a polyether (e.g., PEG), a polyacrylate or an ester thereof, a polyacrylamide, a polyester (e.g., polylactide, polyglycolic), a polyanhydride, a polyvinyl alcohol, a polysaccharide, a poly(N-vinylpyrrolidone), a polyoxazoline, a poly(amino acid), or any combination or a co-polymer thereof.

In some embodiments, the biocompatible and/or biodegradable polymer or oligomer is hydrophilic (e.g., having a water-solubility above 10 g/L).

In some embodiments, the oligomer comprises between 2 and 15, between 2 and 5, between 2 and 10, between 2 and 7, between 5 and 15, between 3 and 8 repeating units, including any range in between, wherein the repeating unit is as described hereinabove. In some embodiments, the oligomer comprises or consists essentially of repeating units having the same chemical composition. In some embodiments, the oligomer comprises or consists essentially of chemically distinct repeating units (e.g., in a form of a copolymer, a random copolymer, a block-copolymer, etc.).

In some embodiments, the oligomer is characterized by an average molecular weight between 30 and 1000 Da, between 30 and 100 Da, between 30 and 200 Da, between 30 and 300 Da, between 30 and 400 Da, between 30 and 500 Da, between 30 and 600 Da, between 30 and 700 Da, between 30 and 800 Da, between 30 and 900 Da, between 100 and 300 Da, between 50 and 350 Da between 100 and 500 Da between 150 and 500 Da between 100 and 900 Da including any range in between.

In some embodiments, the oligomer is or comprises a plurality of ethylene oxide repeating units, i.e., polyethylene glycol (PEG), or a plurality of amino acid residues (i.e., a peptide, such as a random polyamino acid, or a protein).

In some embodiments, the spacer is or comprises:

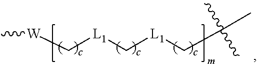

wherein a wavy bond represents an attachment point to the macromolecule; wherein c is an integer independently selected from 0 to 10; m is an integer ranging between 1 and 5 (e.g., 1, 2, 3, 4, or 5 including any range between); each L1 independently represents a bond, or is selected from —N—C(═O)—, —C(═O)N—, —S—S—, —S—, —NR′—, —O—, C(═O), C(═NH), C(═S), Y—(C₀₋₁₀)alkyl-X′—(C₀₋₁₀)alkyl-Y, C1-C10 linear or cyclic alkyl, and a click reaction product, including any combination thereof; wherein each X′ and Y is absent or is independently selected from a heteroatom, an oligomer, the click reaction product, —CONR′—, —CNNR′—, —CSNR′—, —NC(═O)O—, —NC(═S)O—, —NC(═S)N—, —SO2-, —SO—, —SR′, —C(═O)—, —OC(═O)—, —OC(═O)O—, —OC(═S)O—, and —OC(═S)N—; and wherein W represents the click reaction product or a derivative thereof. In some embodiments, the spacer is as disclosed above, wherein W represents a moiety obtained by reacting T with the target associating group). In some embodiments, W is succinimide-thioether, or succinimidyl. A click reaction product derivative encompasses click reaction product without a moiety derived from one of the reactants involved in the click reaction. For example, a succinimide-thioether derivate, as used herein, is succinimidyl. In some embodiments, the spacer is as described above being at least 1 C—C bond long. In some embodiments, the spacer is as described above, wherein at least one c is not 0.

In some embodiments, the spacer is

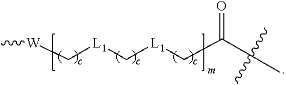

In some embodiments, the spacer is

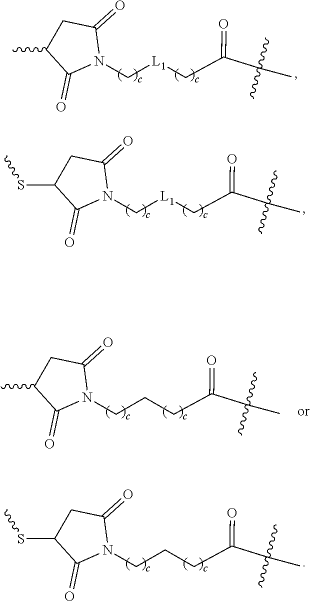

In some embodiments, the conjugate of the invention is represented by Formula I, wherein R2 is C6-alkylamine, R1 is

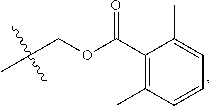 [SMILES: *COC(=O)c1c(C)cccc1C]

### R5 is:

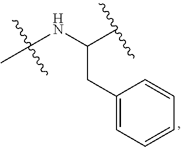 [SMILES: *NC(*)Cc1ccccc1]

and L is or comprises:

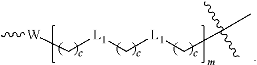

In some embodiments, the conjugate of the invention is represented by Formula IV:

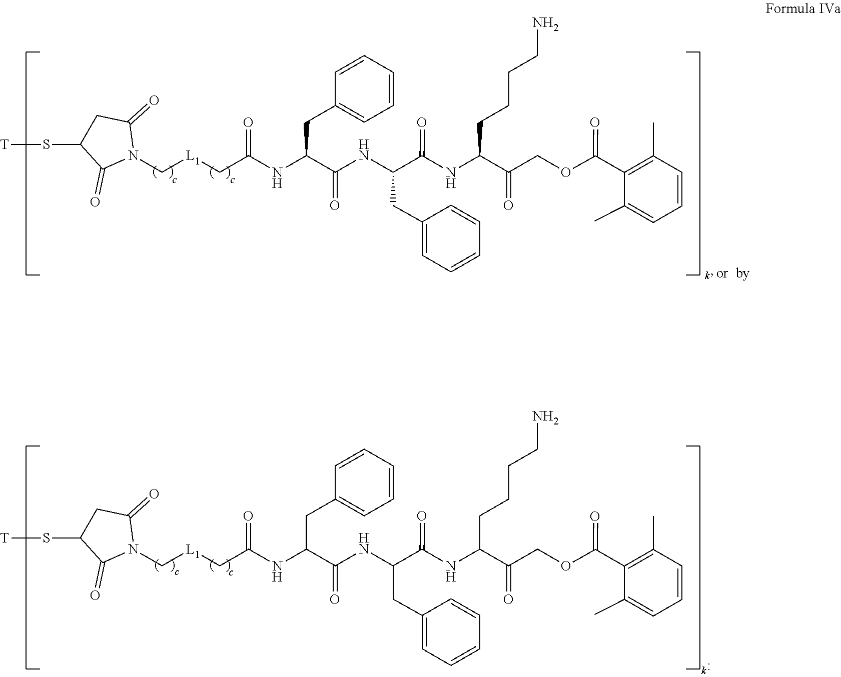

wherein T, c, and L1 are as disclosed above. In some embodiments, the conjugate of the invention is represented by Formula IV, wherein L1 is absent (i.e., is a bond) and wherein at least one c is not 0.

In some embodiments, the conjugate of the invention is represented by Formula V:

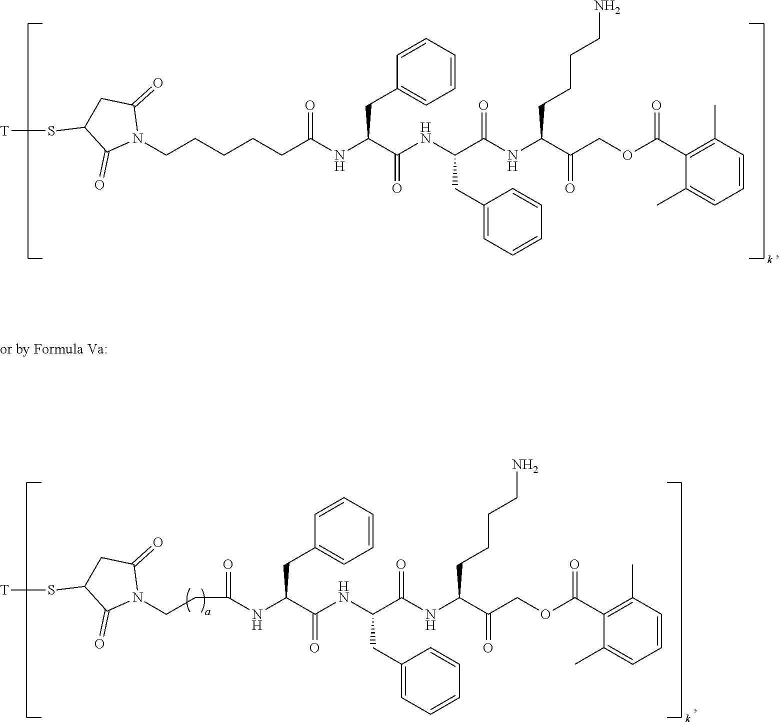

wherein a in integer ranging between 1 and 10.

As used herein, the terms “peptide”, “polyaminoacid”, “polypeptide” and “protein” are used interchangeably and refer to a polymer of amino acid residues.

The terms “peptide”, “polyaminoacid”, “polypeptide” and “protein” as used herein encompass native peptides, peptide derivatives such as beta peptides, peptidomimetics (typically including non-peptide bonds or other synthetic modifications,) and the peptide analogs peptoids and semi-peptoids or any combination thereof. In another embodiment, the terms “peptide”, “polyaminoacid” and “protein” apply to amino acid polymers in which at least one amino acid residue is an artificial chemical analog of a corresponding naturally occurring amino acid.

The term “derivative” or “chemical derivative” includes any chemical derivative of the polypeptide having one or more residues chemically derivatized (or chemically modified) by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine (O) may be substituted for lysine.

In addition, a peptide derivative can differ from the natural sequence of the peptide of the invention by chemical modifications including, but are not limited to, terminal —NH2 acylation, acetylation, or thioglycolic acid amidation, and by amidation of the terminal and/or side-chain carboxy group, e.g., with ammonia, methylamine, and the like. Peptides can be either linear, cyclic, or branched and the like, having any conformation, which can be achieved using methods known in the art.

The term “amino acid” as used herein means an organic compound containing both a basic amino group and an acidic carboxyl group. Included within this term are naturally occurring amino acids, protected amino acids (e.g., comprising one or more protecting groups at the carboxyl, at the amine, and/or at the side chain of the amino acid), unusual, non-naturally occurring amino acids (such as D-amino acids), as well as amino acids which are known to occur biologically in free or combined form but usually do not occur in proteins. Included within this term are modified and unusual amino acids, such as those disclosed in, for example, Roberts and Vellaccio (1983) The Peptides. 5:342-429. Modified, unusual or non-naturally occurring amino acids include, but are not limited to, D-amino acids, hydroxylysine, 4-hydroxyproline, N-Cbz-protected aminovaleric acid (Nva), ornithine (O), aminooctanoic acid (Aoc), 2,4-diaminobutyric acid (Abu), homoarginine, norleucine (Nle), N-methylaminobutyric acid (MeB), 2-naphthylalanine (2Np), aminoheptanoic acid (Ahp), phenylglycine, β-phenylproline, tert-leucine, 4-aminocyclohexylalanine (Cha), N-methyl-norleucine, 3,4-dehydroproline, N,N-dimethylaminoglycine, N-methylaminoglycine, 4-aminopipetdine-4-carboxylic acid, 6-aminocaproic acid, trans-4-(aminomethyl)-cyclohexanecarboxylic acid, 2-, 3-, and 4-(aminomethyl)benzoic acid, 1-aminocyclopentanecarboxylic acid, 1-aminocyclopropanecarboxylic acid, cyano-propionic acid, 2-benzyl-5-aminopentanoic acid, Norvaline (Nva), 4-O-methyl-threonine (TMe), 5-O-methyl-homoserine (hSM), tert-butyl-alanine (tBu), cyclopentyl-alanine (Cpa), 2-amino-isobutyric acid (Aib), N-methyl-glycine (MeG), N-methyl-alanine (MeA), N-methyl-phenylalanine (MeF), 2-thienyl-alanine (2Th), 3-thienyl-alanine (3Th), O-methyl-tyrosine (YMe), 3-Benzothienyl-alanine (Bzt) and D-alanine (DAI).

The term “polyaminoacid” further encompasses random polymers (i.e., devoid of a specific amino acid sequence within the entire composition and include a random population of polymers of different lengths and of different sequences) and polypeptides having a specific amino acid sequence). The terms “peptide sequence” and “amino acid sequence” are used herein interchangeably. In some embodiments, the peptide sequence is or comprises D-amino acid sequence. In some embodiments, at least 70%, at least 80%, at least 90%, at least 95% of the amino acids within the peptide sequence are in D-configuration. In some embodiments, the amino acids within the peptide sequence are in D-configuration.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜ Fc fusions and sc Fv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the antibody is a single chain antibody (ScFv). In some embodiments, the antibody is a single domain antibody. In some embodiments, the antibody is a camelid antibody.

Thus, without being limited to a specific target a composition of the invention can be known to comprise these molecules as the T-moiety. The structure of antibodies is well known and though a skilled artisan may not know to what target an antibody binds merely by its CDR sequences, the general structure of an antibody and its antigen binding region can be recognized by a skilled artisan.

In some embodiments, the antibody binds to an antigen present on the surface of a target cell. In some embodiments, the antigen is a protein. In some embodiments, the antigen is a peptide. In some embodiments, the peptide is a peptide of an infectious agent. In some embodiments, an infectious agent is a pathogen. In some embodiments, the infectious agent is a virus. In some embodiments, the infectious agent is a bacterium. In some embodiments, the virus is SARS-Cov-2. In some embodiments, the protein is a receptor. In some embodiments, the receptor is specific to the target cell. In some embodiments, the receptor characterizes the target cell. In some embodiments, the antigen is a cancer specific antigen. In some embodiments, a cancer specific antigen is a tumor specific antigen. Antibodies that bind to infectious agent peptides, tumor specific antigens and tissue/cell type specific receptors are well known in the art. Any such antibody can be used as the T-moiety.

In some embodiments, the target cell is a disease cell. In some embodiments, the target cell is a cancerous cell. In some embodiments, the target cell is an infected cell. In some embodiments, the target cell is of a tissue or cell type of the disease. In some embodiments, the target cell is treatable by cathepsin inhibition. In some embodiments, the target cell is treatable by a method of the invention.

The term “polynucleic acid” is well known in the art. The terms “polynucleic acid” and “polynucleotide” are used herein interchangeably. A “polynucleic acid” as used herein will generally refer to a polynucleotide and/or a molecule (i.e., a strand and or double strand) comprising DNA, RNA, a synthetic analog of RNA, a mimetic thereof or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The term “polynucleic acid molecule” includes but is not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating polynucleic acids, fragments of genomic DNA or RNA, degraded nucleic acids, ribozymes, viral RNA or DNA, polynucleic acids of infectious origin, amplification products, modified nucleic acids, plasmidical or organellar nucleic acids and artificial nucleic acids such as oligonucleotides.

As used herein, the term “oligonucleotide” refers to a short (e.g., no more than 100 bases), chemically synthesized single-stranded DNA or RNA molecule. In some embodiments, oligonucleotides are attached to the 5′ or 3′ end of a nucleic acid molecule, such as by means of ligation reaction.

In some embodiments, the polynucleotide comprises or consists of RNA. The polynucleotide comprises or consists of a messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The basic components of an mRNA molecule typically include at least one coding region, a 5′ untranslated region (UTR), a 3′ UTR, a 5′ cap and a poly-A tail. Polynucleotides may function as mRNA but can be distinguished from wild-type mRNA in their functional and/or structural design features which serve to overcome existing problems of effective polypeptide expression using nucleic-acid based therapeutics.

In some embodiments, the polynucleic acid has one or more chemical modifications to the backbone or side chains. In some embodiments, the polynucleic acid has at least one locked nucleotide, and/or has a phosphorothioate backbone.

Non-limiting examples of polynucleic acids useful according to the herein disclosed invention include, but are not limited to: antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), antagomirs, peptide nucleic acids (PNAs), ribozymes (catalytic RNA molecules capable to cut other specific sequences of RNA molecules) and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a microRNA (miRNA); a small, temporal RNA (stRNA); or a short, hairpin RNA (shRNA); small RNA-induced gene activation (RNAa); small activating RNAs (saRNAs), or combinations thereof.

In some embodiments, the polynucleotide is chemically modified. In some embodiments, the chemical modification is a modification of a backbone of the polynucleotide. In some embodiments, the chemical modification is a modification of a sugar of the polynucleotide. In some embodiments, the chemical modification is a modification of a nucleobase of the polynucleotide. In some embodiments, the chemical modification increases stability of the polynucleotide in a cell. In some embodiments, the chemical modification increases stability of the polynucleotide in vivo. In some embodiments, the chemical modification increases the stability of the polynucleotide in vitro, such as, in the open air, field, on a surface exposed to air, etc. In some embodiments, the chemical modification increases the polynucleotide's ability to induce silencing of a target gene or sequence, including, but not limited to an RNA molecule derived from a pathogen or an RNA derived from a plant cell, as described herein. In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, a constrained ethyl backbone, an alternating locked nucleic acid backbone, a phosphorothioate backbone, N3′—P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, ligand-conjugated antisense, and a combination thereof.

As used herein, the term “oligonucleotide” refers to a molecule comprising 3-180 bases. In another embodiment, the term “oligonucleotide” refers to a molecule comprising 5-100, 5-200, 5-300, 5-500, 5-700, 5-1000, 20-100, 20-1000, 50-200, 50-500, 50-1000, 50-100, bases, including any range between.

The term “fluorophore” encompasses a fluorescent dye, capable of emitting fluorescent light in the visible and/or NIR range. Exemplary fluorophore is selected from, without being limited thereto, cyanine dye (e.g., non-sulfonated cyanins such as Cy3, Cy5, Cy7, Cy 3.5, Cy 5.5; sulfonated caynins such as sulfo-Cy3, sulfo-Cy5, or sulfo-Cy7) fluorescein, diacetylfluorescein, dipivaloyl Oregon green, tetramethylrhodamine, coumarin-dye, Rhodamine-dye (and Rhodamine silicone derivatives), Alexa Fluor-dyes, and BODIPY-dye.

### Precursor

In another aspect, there is provided a compound represented by Formula D:

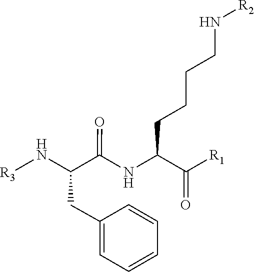

wherein: R1 is an electrophilic functionality selected to permit binding to a protease, R2 is H or a functionality selected from amine and ammonium functionalities, and R3 is an electrophilic side chain.

In some embodiments, R1 is an electrophilic moiety being different from H. In some embodiments, R1 is as disclosed hereinabove. In some embodiments, R1 is α,β-unsaturated carbonyl.

In some embodiments, R2 is a functionality that comprises an amine or an ammonium group. As used herein, the amine is a group of the structure R—NH₂, R—NH—R′ or R—N(R′)—R″, namely a primary or a secondary or a tertiary amine, wherein the N atom is neutral, and wherein each of R, R′ and R″ are same or different and wherein at least one of R, R′ and R″ is a point of connectivity to the NH group to which R2 is substituted. The ammonium group is an amine wherein the amine atom is charged.

The amine or ammonium group may be a cyclic or a non-cyclic functionality. The amine or ammonium may be an end of chain group or an inner chain or inner ring functionality.

In some embodiments, in a compound of formula (D), R2 is H.

In some embodiments, in a compound of formula (D), R2 is an alkyl amine, such as C1-C20NH₂, C1-C20NH—R, C1-C20N(R)R′, or C1-C20NR(R′)(R″), wherein each of R, R′ and R″ may be same or different and may be optionally selected from C1-C10alkyl, C6-C10aryl, Cy5 and others.

In some embodiments, in a compound of formula (D), R2 is Cy5 having the structure:

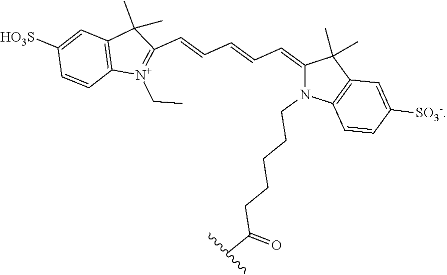

In some embodiments, a compound of formula (D) is a compound of formula

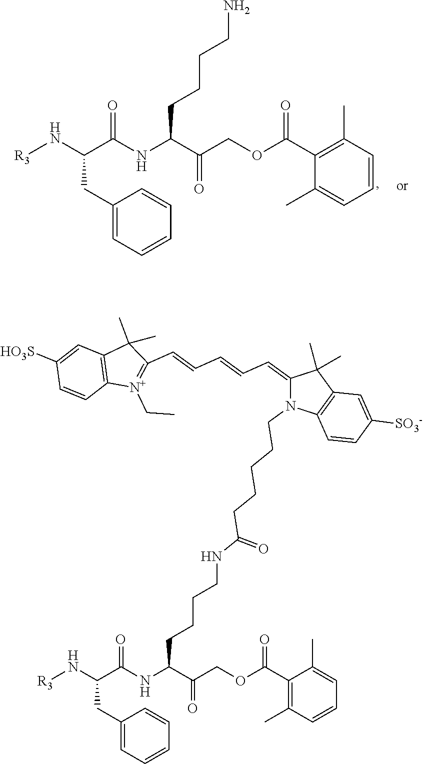

wherein
R3 is as defined herein.

Where any of the functionalities provided in compounds of the invention are charges (namely bearing a positive or negative charge), the charge is typically neutralized by presence or another opposite charge, borne by a counterion or another atom present in the compound.

In each compound of formulae A-D, R3 may be any electrophilic moiety capable of associating, e.g., via covalent bonding, to a molecule or functionality that is capable of associating to a predetermined component in a sample, e.g., cell component, tissue component, enzyme, antibody, drug entity etc. In some embodiments, R3 is or comprises an amino acid or a peptide. In some embodiments, the amino acid is any of the amino acids known in the art, e.g., natural amino acids. In some embodiments, the peptide comprises at least one natural amino acid.

In some embodiments, R3 comprises an aromatic amino acid. In some embodiments, R3 is alanine or phenyl alanine. In some embodiments, R3 is of a structure AA-R4, wherein AA is an amino acid or a peptide and R4 is a target associating group. In some embodiments, R3 is of a structure AA-L-R4, wherein AA is an amino acid or derivative or non-natural amino acid, R4 is a target associating group and L is a spacer, optionally selected from a C1-C6alkylene, C6-C10arylene and other non-labile linker moieties, including polyethylene glycol linkers, e.g., [CH2-CH2-O]n.

In some embodiments, the compound is represented by Formula (E):

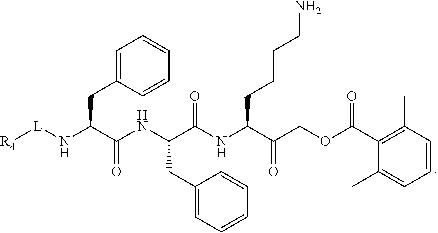

In some embodiments, in the compound of Formula E, R4 is an electrophilic functionality capable of interacting with a chemical functionality such as a thiol, an amine, a hydroxide or a nucleophilic group. The group may be selected from a displacement atom (a leaving group), a carbonyl group, unsaturated carbonyl functionalities (e.g., a double bond), cyano and others.

In some embodiments, spacer L is as disclosed hereinabove. In some embodiments, spacer L is absent.

In some embodiments, R3 is

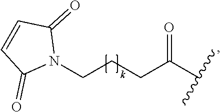

wherein k is between 0 and 4.

In some embodiments, R4 is

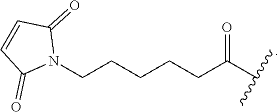 [SMILES: *C(=O)CCCCCN1C(=O)C=CC1=O]

In some embodiments, R3 is

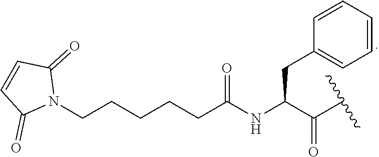 [SMILES: *C(=O)[C@H](Cc1ccccc1)NC(=O)CCCCCN1C(=O)C=CC1=O]

In some embodiments, in compound of formula (A), R1 is

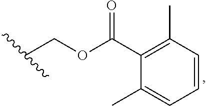 [SMILES: *COC(=O)c1c(C)cccc1C]

### R2 is H and R3 is

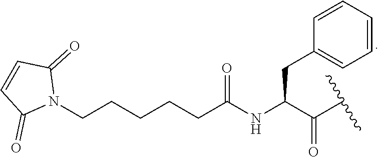 [SMILES: *C(=O)[C@H](Cc1ccccc1)NC(=O)CCCCCN1C(=O)C=CC1=O]

The compounds of formulae D-E have cathepsin inhibition activity, preferably cysteine cathepsin inhibition activity, more preferably cathepsin B, L and S inhibition activities, while being essentially intracellular impermeable.

The compounds of formulae D-E can have two functions in the context of the invention. First, as they are cathepsin inhibitors that do not enter cells and are not toxic to cells, they can be used to inhibit cathepsin, (preferably cysteine cathepsins, more preferably cathepsin B, L and S) in the extracellular matrix space without causing cell death.

This extracellular inhibition may be used to treat diseases wherein a clinical beneficial effect is evident by inhibition of cathepsin activity in the extracellular space. Cathepsins are emerging as important players in the extracellular space, and the paradigm is shifting from the degrading enzymes to the enzymes that can also specifically modify extracellular proteins. In pathological conditions, the activity of cathepsins is often dysregulated, resulting in their overexpression and secretion into the extracellular space. This is typically observed in cancer and inflammation, inflammation that often accompanies different diseases including cancer, arthritis, cardiovascular disease, and bone and joint disorders as a consequence of dysregulated localization, activation or transcription, as well as inhibitor imbalance. Accordingly, cathepsins have been found to be involved in the processing of cytokines and chemokines, thereby representing an important bridge between inflammation and diseases like cancer and psoriasis.

Thus, the present invention further encompasses a pharmaceutical composition comprising a pharmaceutically acceptable carrier and as an active ingredient the compound of formulae D-E, or VI-VII.

The pharmaceutical composition is for the treatment of a disease, disorder or pathological condition wherein a beneficial clinical effect is achieved by the inhibition of cathepsin (in particular cathepsin B, L or S) in the extracellular space. In some embodiments, disease, disorder or pathological condition associated with cathepsin activity in the extracellular space is selected from osteoarthritis (cathepsin B, K, L, S), cancer (cathepsin S), adipogenesis (cathepsin S), intestinal trauma (cathepsin S), osteoporosis (cathepsin K), rheumatized arthritis (cathepsin K), lung fibrosis (B, K, L, S), cardiovascular disease (B, K, L, S), neuropathic pain (cathepsin S).

In another aspect of the invention, there is provided a compound represented by Formula VI:

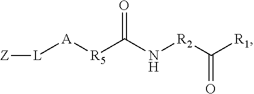

wherein R1, R2, R5 are as disclosed hereinabove (e.g., in Formulae I-V); wherein L is a spacer; and wherein Z is an electrophilic functionality having a reactivity to a thiol group, an amine group, or both.

In some embodiments, the compound of the invention is represented by Formula VI, wherein R1 comprises any one of: chloromethyl ketone, acyloxymethyl ketone, a Michael acceptor, phosphonate, cyano group, or

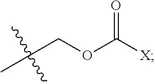

wherein X is selected from a substituted or unsubstituted alkyl; a substituted or unsubstituted alkenyl, a substituted or unsubstituted aryl, a substituted or unsubstituted heteroaryl; R2 comprises a substituted or unsubstituted alkylamine, or —[C(D′)₂]ₙ—; wherein each D′ is independently H, a substituent, or an optionally substituted C1-C20 alkylamine; and wherein at least one D′ is the optionally substituted C1-C20 alkylamine; R5 comprises

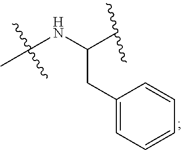 [SMILES: *NC(*)Cc1ccccc1]

A represents at least one amino acid residue; n is an integer ranging between 1 and 3; L represents a spacer; and Z is an electrophilic functionality having a reactivity to a thiol group, an amine group, or both.

In some embodiments, the compound of the invention is represented by Formula VI, wherein Z has a reactivity to a functional group selected from a thio group, an amino group, 1,3-nitrone, azide, a diene, succinimide, alpha, beta unsaturated carbonyl, and an active ester. In some embodiments, Z has a reactivity to a small molecule or to a macromolecule (i.e., variable T) comprising the functional group. In some embodiments, Z has a reactivity to the functional group of an antibody.

In some embodiments, Z is or comprises maleimide; an active ester (e.g., thio-ester, a pentofluorophenyl ester, a N-hydroxysuccinimide ester, hydroxybenzotriazole ester, etc.); alpha,beta unsaturated carbonyl; 1,3-nitrone; thiol; amine; azide; alkyne; diene; alkene; tetrazole, or any combination thereof. In some embodiments, Z is or comprises an acyl halide, a chloroformate, an anhydride, an aldehyde, an epoxide, an isocyanate, an isothiocyanate, a maleimide, a carbonate, a sulfonyl chloride, iodoacetamide, an acyl azide, an imidoester, a vinyl sulfone, ortho-pyridyl-disulfide, or any combination thereof.

The term “active ester” refers to ester with enhanced reactivity (fast kinetics) towards a nucleophilic attack, as compared to a regular alkyl ester. Active esters react with nucleophiles at room temperature, resulting in almost quantitative amide bond formation. The active esters have alcohol component inducing greater electron withdrawing effect, as compared to a regular alkyl ester. Withdrawal of electrons enhances the electrophilic character of the carbonyl carbon and thereby facilitates the formation of the tetrahedral intermediate with the nucleophile. Usually, the alcohol component of an active ester is a better leaving group, as compared to an alcohol of the regular alkyl ester. As used herein, the term “active ester” refers to a storage stable compound.

In some embodiments, Z is maleimide.

In some embodiments, R2 is an optionally substituted C1-C20alkyl-NB′B′, wherein B′ is R′ or a fluorophore; wherein X is

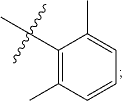 [SMILES: *c1c(C)cccc1C]

and wherein A comprises an aromatic amino acid residue, alanine residue, or both.

In some embodiments, the compound of the invention is represented by Formula VI, wherein R1 is

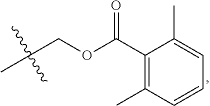 [SMILES: *COC(=O)c1c(C)cccc1C]

wherein R5 is:

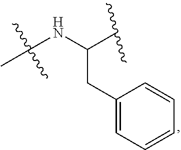 [SMILES: *NC(*)Cc1ccccc1]

and wherein R2 is C1-C20 alkylamine (e.g., C4-alkyl-NH2, C5-alkyl-NH2).

In some embodiments, the compound of the invention is represented by Formula by Formula VII:

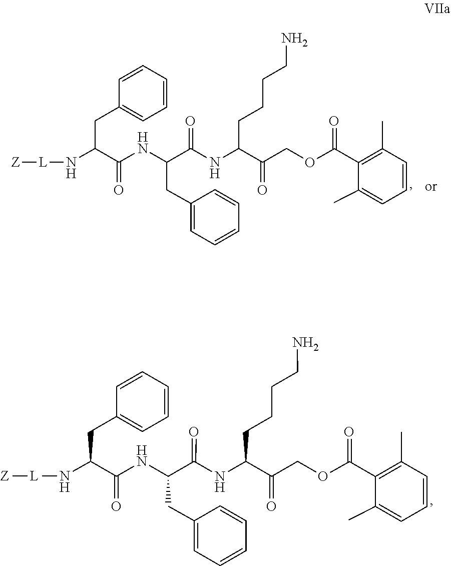

wherein Z and L are as disclosed above. In some embodiments, the compound of the invention is represented by Formula VI or VII, wherein Z is maleimide.

In some embodiments, the compound of the invention is represented by Formula VI or VII, wherein L is or comprises a linear or a branched chain. In some embodiments, L comprises a backbone comprising a linear or a branched chain. In some embodiments, L comprises a cyclic backbone.

In some embodiments, L has a MW less than 1,000 Da, less than 900 Da, less than 800 Da, less than 700 Da, less than 600 Da, less than 500 Da, less than 400 Da, less than 300 Da, less than 200 Da, less than 100 Da, or between 100 and 1000 Da, between 800 and 2,000 Da, between 800 and 1,000 Da, between 800 and 1,500 Da, between 800 and 900 Da, between 900 and 1,000 Da, between 1,000 and 1,100 Da, between 1000 and 1,200 Da, between 800 and 1,200 Da, between 1,000 and 3,000 Da, including any range between. In case where the composition of the invention comprises conjugates containing spacers characterized by different molecular weights, the term “MW” as used herein, refers to an average molecular weight of the spacers within the composition.

In some embodiments, the compound of the invention is represented by Formula VI or VII, wherein the spacer is between 1 and 50, between 1 and 100, between 2 and 100, between 2 and 80, between 2 and 60, between 5 and 50, between 10 and 50, between 10 and 40, between 2 and 30, between 2 and 20, between 2 and 10, between 1 and 5, between 5 and 10, between 5 and 15, between 5 and 25, between 5 and 50 single C—C bonds long, including any range in between. In some embodiments, the spacer is characterized by an average molecular weight between 300 and 3,000 Da, and is between 2 and 50, between 2 and 10, between 2 and 20, between 5 and 30 C—C bonds long, including any range between.

In some embodiments, the compound of the invention is represented by Formula VI or VII, wherein the spacer comprises a polymer or oligomer, wherein polymer or oligomer is as described above (in Conjugates section).

In some embodiments, the compound of the invention is represented by Formula VI or VII, wherein the spacer is or comprises:

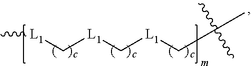

wherein a wavy bond represents an attachment point to Z; wherein c is an integer independently selected from 0 to 10; m is an integer ranging between 1 and 5 (e.g., 1, 2, 3, 4, or 5 including any range between); each L1 independently represents a bond, or is selected from —N—C(═O)—, —C(═O)N—, —S—S—, —S—, —NR′—, —O—, C(═O), C(═NH), C(═S), Y—(C₀₋₁₀)alkyl-X′—(C₀₋₁₀)alkyl-Y, C1-C10 linear or cyclic alkyl, and a click reaction product, including any combination thereof; wherein each X′ and Y is absent or is independently selected from a heteroatom, an oligomer, the click reaction product, —CONR′—, —CNNR′—, —CSNR′—, —NC(═O)O—, —NC(═S)O—, —NC(═S)N—, —SO2-, —SO—, —SR′, —C(═O)—, —OC(═O)—, —OC(═O)O—, —OC(═S)O—, and —OC(═S)N—.

In some embodiments, the spacer is as described above being at least one C—C bond long. In some embodiments, the spacer is as described above, wherein at least one c is not 0.

In some embodiments, the spacer is

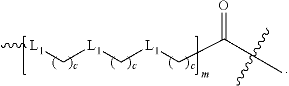

In some embodiments, the spacer is

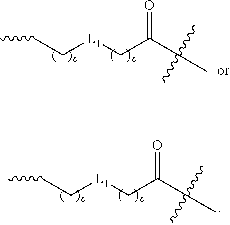

In some embodiments, the compound of the invention is represented by Formula VIIb:

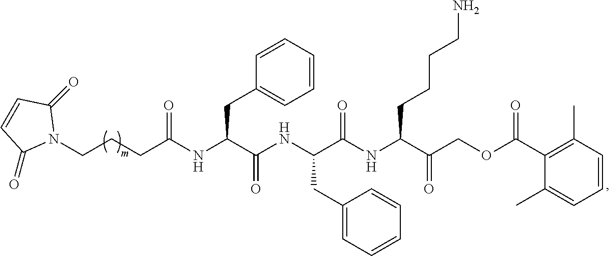

wherein a is in integer range in between 1 and 10, between 1 and 5, between 2 and 5, including any range between.

In some embodiments, the compound

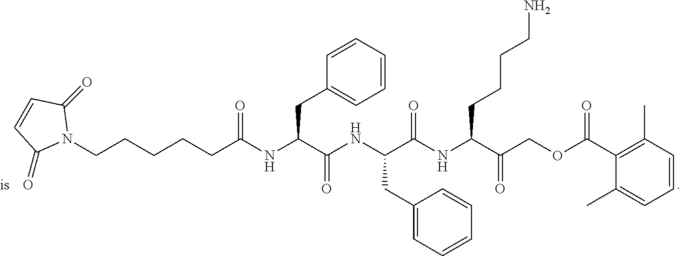

In some embodiments, the compound disclosed herein (i.e., the compound of Formulae VI-VIIb) is a precursor of the conjugate of the invention. In some embodiments, the compound disclosed herein (precursor) is configured to react (e.g., by click reaction) with the T variable disclosed herein, so as to obtain the conjugate of the invention.

In some embodiments, the conjugate and/or the compound of the invention has a pharmaceutical grade purity, i.e., the conjugate and/or the compound is characterized by a chemical purity of at least about 90%, at least about 95%, greater than 95%, or greater than 99%, greater than 97%, or between 95 and 100%, wherein purity is as determined by conventional analytical methods, such as LC-MS, HPLC, GC-MS, etc. In some embodiments, the conjugate of the invention is devoid of unreacted T molecules, or unreacted precursors. In some embodiments, the conjugate of the invention is devoid of un impurity above 0.5%, or above 1%, as determined by HPLC.

The term “substituent”, as used herein encompasses one or more substituents (e.g., 1, 2, 3, 4, 5, or 6), each independently selected from the group consisting of: —OH, oxo, carbonyl, halogen, —OR′, —NO₂, —CN, —CONH₂, —CONR′₂, —CNNR′₂, —CSNR′₂, —CONH—OH, —CONH—NH₂, —NHCOR′, —NHCSR′, —NHCNR, —NC(═O)OR′, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, —NR′R′, NR′NR′₂, C1-C6 haloalkyl, optionally substituted C1-C6 alkyl, —NH₂, —NH(C1-C6 alkyl), —N(C1-C6 alkyl)₂, C1-C6 alkoxy, C1-C6 haloalkoxy, hydroxy (C1-C6 alkyl), hydroxy (C1-C6 alkoxy), alkoxy (C1-C6 alkyl), alkoxy (C1-C6 alkoxy), C1-C6 alkyl-OR′, C1-C6 alkyl-NR′₂, C1-C6 alkyl-SR′, —CONH(C1-C6 alkyl), —CON(C1-C6 alkyl)₂, —CO₂H, —CO₂R′, —OCOR, —OCOR′, —OC(═O)OR′, —OC(═O)NR′, —OC(═S)OR′, —OC(═S)NR′, optionally substituted aryl, optionally substituted heteroaryl, optionally substituted cycloalkyl, optionally substituted heterocyclic alkyl, C2-C6 alkenyl, C2-C6 alkynyl, (C3-C6 cycloalkyl) (C0-C3 alkyl), (3- to 6-membered monocyclic heterocycle) (C0-C3 alkyl), (6- to 10-membered monocyclic or bicyclic aryl) (C0-C3 alkyl), (5- to 10-membered monocyclic or bicyclic heteroaryl) (C0-C3 alkyl), R′C(O)—O—(C0-C3 alkyl)-, R′C(O)—(R′N)—(C0-C3 alkyl)-, R'S(O)₂—O—(C0-C3 alkyl)-, R'S(O)₂—(R′N)—(C0-C3 alkyl)-, R′C(O)—, R'S(O)—, and R'S(O)₂—; wherein each R′ is independently selected from hydrogen, C1-C3 alkyl, C1-C6 haloalkyl, C2-C6 alkenyl, C2-C6 alkynyl, (C3-C7 cycloalkyl)-(C0-C3 alkyl)-, (4- to 6-membered heterocycle)-(C0-C3 alkyl)-, (5- to 10-membered monocyclic or bicyclic aryl)-(C0-C3 alkyl)-, (5- to 10-membered monocyclic or bicyclic heteroaryl)-(C0-C3 alkyl)-, OR′, —CONH₂, —CONR′₂, —CNNR′₂, —CSNR′2, —CONH—OH, —CONH—NH₂, —NHCOR′, —NHCSR′, —NHCNR, —NC(═O)OR′, —NC(═O)NR′, —NC(═S)OR′, —NC(═S)NR′, —NR′NR′2, and —NNR′, each of which may be optionally substituted as allowed by valency.

In some embodiments, the compounds described herein are chiral compounds (i.e., possess an asymmetric carbon atom). In some embodiments, diastereomers, geometric isomers and individual isomers are encompassed within the scope of the present invention. In some embodiments, a chiral compound described herein is in the form of a racemic mixture. In some embodiments, a chiral compound is in the form of a single enantiomer, with an asymmetric carbon atom having the R configuration. In some embodiments, a chiral compound is in the form of a single enantiomer, with an asymmetric carbon atom having the S configuration as described hereinabove.

In some embodiments, a chiral compound is in the form of a single enantiomer with enantiomeric purity of more than 70%. In some embodiments, a chiral compound is in the form of a single enantiomer with enantiomeric purity of more than 80%. In some embodiments, a chiral compound is in a form of a single enantiomer with enantiomeric purity of more than 90%. In some embodiments, a chiral compound is in the form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the compounds described herein can exist in unsolvated form as well as in solvated form, including hydrated form. In general, the solvated form is equivalent to the unsolvated form and is encompassed within the scope of the present invention. Certain compounds of the present invention may exist in multiple crystalline or amorphous forms. In general, all physical forms are equivalent for the uses contemplated by the present invention and are intended to be within the scope of the present invention.

The term “solvate” refers to a complex of variable stoichiometry (e.g., di-, tri-, tetra-, penta-, hexa-, and so on), which is formed by a solute (the conjugate described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

Unless otherwise indicated, structures depicted herein are also meant to include all isomeric (e.g., enantiomeric, diastereomeric, geometric, conformational, and rotational) forms of the structure. For example, the R and S configurations for each asymmetric center, (Z) and (E) double bond isomers, and (Z) and (E) conformational isomers are included in this invention. As would be understood to one skilled in the art, a substituent can freely rotate around any rotatable bond. Therefore, single stereochemical isomers as well as enantiomeric, diastereomeric, geometric, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a hydrogen by 18F, or the replacement of a carbon by a 13C- or 14C-enriched carbon are within the scope of this invention. Such compounds are useful, for example, as imaging probes.

In some embodiments, the compound of the invention includes any salt, any solvate, any hydrate, any stereoisomer, any isotope (e.g., a deuterated compound), and/or any derivative (e.g., a biologically active derivative) of any of the compounds or of the Formulae disclosed herein.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one embodiment, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively, or in addition, isotopes of carbon, e.g., ¹³C and ¹⁴C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

### Kit and Process of Manufacture

In another aspect there is provided a kit comprising the compound of the invention (i.e., precursor), and a targeting molecule having reactivity to the compound (i.e., reactivity to Z moiety). In some embodiments, the targeting molecule is a small-, or macro-molecule as described for T variable. The terms “compound of the invention” and “precursor” are used herein interchangeably.

In some embodiments, the small-, or macro-molecule comprise a reactive group having reactivity to Z moiety, wherein the reactive group comprises a thio group, an amino group, 1,3-nitrone, azide, diene, tetrazine, or any combination thereof. In some embodiments, the targeting molecule comprises at least one thio group. In some embodiments, the targeting molecule is an antibody, or an antigen binding fragment of an antibody.

In some embodiments, the targeting molecule is within a composition in the kit (e.g., a solid composition, or a liquid composition such as a solution, a dispersion, or a suspension).

In some embodiments, a molar ratio between the targeting molecule and the precursor within the kit is between about 1:2 and between about 1:300, between about 1:3 and 1:200, between about 1:10 and 1:200, between about 1:30 and 1:200, between about 1:50 and about 1:200, including any range in between.

In some embodiments, the kit further comprises instructions for reacting the targeting molecule and the precursor under suitable conditions, to obtain a conjugate of the invention. In some embodiments, suitable conditions comprise conditions appropriate for reacting the targeting molecule and the precursor of the invention, such as reaction time (e.g., between 0.1 and 10 h, including any range between), a temperature (e.g., between 5 and 90C, including any range between).

In some embodiments, the kit further comprises a solvent. In some embodiments, the solvent is appropriate for dissolving the polymer and the precursor. In some embodiments, the polymer and the precursor have a solubility within the solvent of at least 0.5 g/L, at least 10 g/L, or between 0.5 and 100 g/l, including any range between. In some embodiments, the solvent is selected from but not limited to an aqueous buffer, water, or an organic solvent.

### Pharmaceutical Composition

In another aspect of the invention disclosed herein, there is provided a pharmaceutical composition comprising the conjugate of the invention, a pharmaceutically acceptable salt thereof or both; and a pharmaceutically acceptable carrier. In some embodiments, the pharmaceutical composition of the invention comprises a therapeutically effective amount of the conjugate of the invention and/or any pharmaceutically acceptable salt and/or derivative thereof. In some embodiments, therapeutically effective amount is sufficient for reduction of at least one symptom, or for substantial reduction in the severity and/or inhibition of the progression of a disease, disorder, or condition as described hereinabove. In some embodiments, the therapeutically effective amount can be determined as described herein.

In some embodiments, there is provided herein a composition comprising one or more conjugates of the invention, including any salt (e.g., a pharmaceutically acceptable salt), any tautomer, and/or any stereoisomer thereof. In some embodiments, the conjugate as described hereinabove is the only active ingredient within the composition of the invention (e.g., pharmaceutical composition).

In some embodiments, the composition of the invention is a pharmaceutical composition comprising at least one conjugate of the invention and a pharmaceutically acceptable carrier, excipient or adjuvant. In some embodiments, the composition of the invention is a pharmaceutical composition comprising at least one conjugate of the invention as a first active ingredient and an additional active ingredient.

In some embodiments, the pharmaceutical composition comprises the compound of the invention and a pharmaceutically acceptable carrier. In some embodiments, the pharmaceutical composition comprises a therapeutically effective amount of the compound of the invention and the pharmaceutically acceptable carrier.

In some embodiments, the pharmaceutical composition is in the form of a combination or of a kit of parts. In some embodiments, the pharmaceutical composition of the invention is for use as a medicament.

For example, the term “pharmaceutically acceptable” can mean approved by a regulatory agency of the Federal or a state government or listed in the U.S. Pharmacopeia or other generally recognized pharmacopeia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for systemic use. In some embodiments, the composition is formulated for systemic administration. In some embodiments, the composition is formulated for intravenous administration. In some embodiments, the composition is formulated for intratumoral administration.

As used herein, the term “carrier” refers to a diluent, adjuvant, excipient, or vehicle with which the active ingredient is administered. Such carriers can be sterile liquids, such as water-based and oils, including those of petroleum, animal, vegetable or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has a sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition comprising the compound/conjugate of the invention is in a unit dosage form. In some embodiments, the pharmaceutical composition is prepared by any of the methods well known in the art of pharmacy. In some embodiments, the unit dosage form is in the form of a tablet, capsule, lozenge, wafer, patch, ampoule, vial or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral or transdermal dosage form.

As used herein, the terms “administering”, “administration”, and like terms refer to any method which, in sound medical practice, delivers a composition containing an active agent to a subject in such a manner as to provide a therapeutic effect. In some embodiments, administering is by an oral administration, a systemic administration or a combination thereof.

In some embodiments, the pharmaceutical composition is administered via oral (i.e., enteral), rectal, vaginal, topical, nasal, ophthalmic, transdermal, subcutaneous, intramuscular, intraperitoneal or intravenous routes of administration. The route of administration of the pharmaceutical composition will depend on the disease or condition to be treated. Suitable routes of administration include, but are not limited to, parenteral injections, e.g., intradermal, intravenous, intramuscular, intratumoral, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer. In some embodiments, administering is systemic administering. In some embodiments, administering is intravenous administering. In some embodiments, administering is intratumoral administering.

In some embodiments, for oral applications, the pharmaceutical composition or is in the form of a tablets or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in the form of a drinkable liquid. In some embodiments, oral application of the pharmaceutical composition or of the kit is in the form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes.

In some embodiments, the pharmaceutical composition is for use in the inhibition of cathepsin activity. In some embodiments, cathepsin activity comprises cysteine cathepsin activity. In some embodiments, cysteine cathepsin is selected from cathepsin B, cathepsin L and cathepsin S. In some embodiments, the pharmaceutical composition is for use in the inhibition of cathepsin S activity.

In some embodiments, cathepsin activity comprises an intracellular cathepsin activity, an extracellular cathepsin activity, or both. In some embodiments, cathepsin activity comprises abnormal activity. In some embodiments, abnormal cathepsin activity comprises increased cathepsin activity as compared to cathepsin activity within a cell or within a tissue of a healthy subject.

In some embodiments, inhibition refers to an inhibition of cathepsin enzymatic activity within at least one cell and/or within a tissue of the subject, wherein inhibition is by at least 10%, at least 20%, at least 50%, at least 70%, at least 80%, at least 90%, at least 95%, at least 99%, compared to an initial enzymatic activity (e.g., enzymatic activity within a cell/tissue without administration of the conjugate/compound of the invention). Each possibility represents a separate embodiment of the invention. In some embodiments, inhibition is at least 20% inhibition. In some embodiments, inhibition is at least 50% inhibition. In some embodiments, inhibition is at least 80% inhibition. In some embodiments, inhibition is at least 90% inhibition.

In some embodiments, inhibition is an irreversible inhibition. In some embodiments, the conjugate and/or compound of the invention is/are irreversible cathepsin inhibitors. In some embodiments, the pharmaceutical composition comprises the conjugate and/or compound of the invention as the therapeutically active ingredient, wherein the conjugate and/or compound of the invention is represented by any Formula or chemical structure disclosed herein and is an irreversible cathepsin inhibitor, wherein cathepsin inhibition is as disclosed hereinbelow.

In some embodiments, the conjugate and/or compound of the invention is/are irreversible cysteine cathepsin inhibitors. In some embodiments, the conjugate and/or compound of the invention is/are irreversible cysteine cathepsin inhibitors; and are further characterized by selectivity to a cysteine cathepsin selected form cathepsin B, cathepsin L and cathepsin S. In some embodiments, the conjugate and/or compound of the invention is/are irreversible cysteine cathepsin inhibitors; and are further characterized by selectivity to cathepsin S. The term “selectivity” in conjunction with cathepsin inhibition refers to a selective binding and inhibition of the cathepsin of interest (e.g., a cysteine cathepsin selected form cathepsin B, cathepsin L and cathepsin S), wherein selective inhibition encompasses at least 5 fold, at least 10 fold, at least 20 fold, at least 50 fold, at least 100 fold, at least 200 fold, at least 500 fold, at least 1000 fold greater selectivity constant to the cathepsin of interest including any range between, as compared to another cathepsin (e.g., a cysteine cathepsin which is not cathepsin B, cathepsin L or cathepsin S).

In some embodiments, IC50 of the conjugate of the invention (i.e., in context of cathepsin inhibition, as disclosed herein) is less than 1000 nM, less than 700 nM, less than 200 nM, less than 500 nM, less than 50 nM, less than 10 nM, including any range between. In some embodiments, the conjugate of the present invention has at least 2 times, at least 3 times, at least 4 times, at least 5 times, at least 10 times lower IC50 value for intracellular cathepsin activity, as compared to the compound of the invention (i.e., unconjugated precursor). In some embodiments, the conjugate or the compound of the invention is characterized by a selective inhibition of cathepsin B, cathepsin L and/or cathepsin S.

The pharmaceutical composition is for the treatment of a disease, disorder or pathological condition wherein a beneficial clinical effect is achieved by the inhibition of cathepsin (in particular cathepsin B, L or S) in the extracellular space.

The present invention further concerns a method for treatment of a disease, disorder or pathological condition wherein a clinical beneficial effect is achieved by inhibiting cathepsin activity in the extracellular space the method comprising: administering to a subject in need of such treatment a therapeutically effective amount of a compound of any one of Formulae VI-VII and D-E.

The present invention further concerns a method of treatment of a diseases, disorder or pathological condition wherein a clinically beneficial effect is evident by inhibiting of intracellular cathepsin activity (such as cysteine cathepsin activity, or specifically B, L or S cathepsin activity); the method comprising: administering to a subject in need of such treatment an effective amount of the conjugate of any one of Formulae I-V and A-C. Typically, such diseases, or conditions are inflammatory and cardiovascular diseases, neurodegenerative disorders, diabetes, obesity, cancer, kidney dysfunction.

In some embodiments, the disease is viral infection. In some embodiments, the inhibition of the intracellular activity prevents viral maturation, propagation, replication, assembly or secretion. A non-limiting example of such a virus is SARS-COV 2 and the targeting agent is an ACE2 receptor binding agents being an antibody or a RGD (receptor binding domain) sequence.

Another non-limiting example of a disease or conditions suitable for treatment by the conjugates of the invention is cancer, in particular lymphoma and in such a case the targeting agent is an antibody that targets the cancer cells such as Rituximab.

A detailed list of diseases wherein a clinically beneficial effect is evident in inhibiting cathepsin activity in target cell type, target tissue, target cell state comprises osteoarthritis (cathepsin B, K, L, S), cancer (cathepsin S), adipogenesis (cathepsin S), intestinal trauma (cathepsin S), osteoporosis (cathepsin K), rheumatized arthritis (cathepsin K), lung fibrosis (B, K, L, S), cardiovascular disease (B, K, L, S), neuropathic pain (cathepsin S).

As some diseases, disorders and pathological conditions (notably cancer and inflammation) can show clinical beneficial effect from inhibition of both intracellular and extracellular cathepsin activity the present invention further concerns a pharmaceutical composition comprising a mixture of: at least one of the compounds of Formulae VI-VII and D-E; and at least one of the compounds of Formulae I-V and A-C.

The present invention further concerns a kit comprising:
A) a container holding at least one of the compounds of Formulae VI-VII and D-E; and
B) a container holding at least one of the compounds of Formulae I-V and A-C; optionally with instruction for administration

The pharmaceutical composition and kit may be for the treatment of a disease, disorder or pathological condition wherein a clinically beneficial effect may be achieved by inhibition of both extracellular cathepsin and intracellular cathepsin in a target cell type, target tissue or target cell state.

The kit may be used in cases where the administration regime, dosing, or timing of the extracellular compounds of formula I, II or III and the target-directed intracellular compounds of III and V are different.

The present invention further concerns a method of treatment of a disease, disorder or, pathological conditions wherein a clinical beneficial effect is achieved by inhibition of both intracellular and extracellular cathepsin activity the method comprising: administering to a subject in need of such treatment at least one of the compounds of Formulae I-V and A-C; and at least one of the compounds of Formulae VI-VII and D-E.

### Methods of Treatment

According to some embodiments, the present invention provides a method for preventing or treating a disease or a disorder associated with cathepsin activity in a subject in need thereof, comprising administering to the subject a therapeutically effective amount of a compound or a pharmaceutical composition as described herein, thereby preventing or treating said disease or said disorder associated with said cathepsin activity in the subject.

In some embodiments, the disease or said disorder is selected from a cell proliferation related disease, an inflammatory disease, a cardiovascular disease, an autoimmune disease, a neurodegenerative disorder, diabetes, obesity, kidney dysfunction, an ocular disease and an infectious disease, including any combination thereof.

In some embodiments, the infectious disease is selected from: a viral disease, a bacterial disease and a parasitic disease. In some embodiments, the disease is a viral disease. In some embodiments, the viral disease is induced by a human pathogenic virus. In some embodiments, the viral disease is induced by a viral infection. In some embodiments, the viral infection is by a respiratory virus. In some embodiments, the viral disease is a respiratory viral disease. In some embodiments the viral infection is a coronavirus infection. In some embodiments the respiratory viral disease is induced by a coronavirus. In some embodiments, the respiratory viral disease is induced by a SARS-COV-2 virus infection.

In some embodiments, respiratory viruses are viruses such as Adenovirus, Coronavirus HKU1, Coronavirus NL63, Coronavirus 229E, Coronavirus OC43, Severe Acute Respiratory Syndrome Coronavirus 2 (SARS COV 2), Human Metapneumovirus, Human Rhinovirus/Enterovirus, Influenza A, Influenza A/H1, Influenza A/H3, Influenza A/H1-2009, Influenza B, Parainfluenza Viruses 1-4, Respiratory Syncytial Virus. In some embodiments the coronavirus is OC43, HKU1 or MERS-COV. In some embodiments, the coronavirus is SARS-CoV-1.

In some embodiments, the disease or disorder is selected from hypertension, Alzheimer's disease, CLN10 Disease, Gaucher Disease, pancreatitis, Papillion-Lefevre syndrome, periodontitis, Parkinson's Disease, Huntington's Disease, dermatitis, CLN13 Disease, diabetes, pycondysostosis, dementia, cancer and autoimmune arthritis. In some embodiments, the disease is a cell proliferation related disease. In some embodiments, the proliferation related disease comprises cancer. In some embodiments, the disease is cancer. In some embodiments, the cancer comprises any one of a metastatic cancer, a solid tumor, and a liquid tumor. In some embodiments, the cancer is a solid cancer. In some embodiments, the cancer is a tumor. In some embodiments, the cancer is PD-L1 positive cancer. In some embodiments, the cancer is a cancer treatable by immunotherapy. In some embodiments, the method further comprises administering at least one immunotherapy treatment to the subject.

In some embodiments, the cathepsin activity comprises an enhanced enzymatic cathepsin activity, wherein enhanced is by at least 10% as compared to enzymatic cathepsin activity within a healthy subject. In some embodiments, enhanced cathepsin activity comprises an increase by at least 10%, at least 50%, at least 100%, at least 200%, at least 500%, at least 1000%, or between 10 and 500%, as compared to cathepsin activity within a cell or within a tissue of a healthy subject, including any range between. Each possibility represents a separate embodiment of the invention. In some embodiments, enhanced cathepsin activity comprises an increase by at least 50%. In some embodiments, enhanced cathepsin activity comprises an increase by at least 80%. In some embodiments, enhanced cathepsin activity comprises an increase by at least 100%.

In some embodiments, administering comprises an administration route selected from intravenous administration, intraperitoneal administration, subcutaneous administration, intratumoral administration or any combination thereof.

In some embodiments, a therapeutically effective amount is sufficient for reducing the enhanced enzymatic cathepsin activity in the subject. In some embodiments, reducing is by at least 10%, at least 20%, at least 30%, at least 50%, at least 100%, at least 200%, at least 300%, at least 1000%, including any range between, as compared to enzymatic cathepsin activity within the same subject before treatment. Each possibility represents a separate embodiment of the invention. In some embodiments, reducing is by at least 10%. In some embodiments, reducing is by at least 50%. In some embodiments, reducing is by at least 80%. In some embodiments, reducing is by at least 90%

In some embodiments, the method further comprising a step preceding the administering step, comprising determining cathepsin levels in the subject, wherein an increase of the levels as compared to a control, is indicative of the subject being suitable for the treating. In some embodiments, the method further comprising a step preceding the administering step, comprising determining an enzymatic activity of a cathepsin in the subject, wherein an increase of the enzymatic activity in the subject compared to a control, is indicative of the subject being suitable for treating. In some embodiments, increase by at least 10%, at least 50%, at least 100%, at least 200%, at least 500%, at least 1000%, or between 10 and 500%, as compared to control (e.g., cathepsin activity within a cell or within a tissue of a healthy subject). Each possibility represents a separate embodiment of the invention. In some embodiments, increase by at least 10%. In some embodiments, increase by at least 50%. In some embodiments, increase by at least 80%. In some embodiments, increase by at least 100%. In some embodiments, the determining is in a sample obtained or derived from the subject. In some embodiments, the sample is a body tissue or a body fluid.

In some embodiments, cathepsin activity comprises a cysteine cathepsin enzymatic activity. In some embodiments, cysteine cathepsin is selected from cathepsin B, cathepsin L and cathepsin S. In some embodiments, determining is determining cathepsin S activity.

According to another aspect, the present invention provides a method for treating or preventing cancer in a subject, comprising administering to the subject a therapeutically effective amount of the compound, the conjugate or a pharmaceutical composition as described herein, thereby treating or preventing cancer in the subject. In some embodiments, the subject is a human subject.

According to another aspect, the present invention provides a method for treating or preventing cancer in a subject, comprising administering to the subject a therapeutically effective amount of the compound, the conjugate or a pharmaceutical composition as described herein and an immunotherapy, thereby treating or preventing cancer in the subject.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition or method herein needs only to reduce the severity of a disease, disorder, or condition, reduce the severity of symptoms associated therewith, or provide improvement to a patient or subject's quality of life. In some embodiments, treating or preventing is treating.

In some embodiments, the subject is a human subject. As used herein the term “subject” refers to an individual, or a patient, which is a vertebrate, e.g., a mammal, including especially a human. In some embodiments, the subject is a human. In some embodiments, the subject is a mammal. In some embodiments, the subject suffers from cancer.

In some embodiments, the method comprises administering the pharmaceutical composition of the invention at least 1 time, at least 2 times, at least 3 times, at least 4 times, at least 5 times, at least 7 times, or at least 10 times per day, or any value and range therebetween. Each possibility represents a separate embodiment of the invention. In some embodiments, the method comprises administering the composition of the invention 1-2 times per day, 1-3 times per day, 1-4 times per day, 1-5 times per day, 1-7 times per day, 2-3 times per day, 2-4 times per day, 2-5 times per day, 3-4 times per day, 3-5 times per day, or 5-7 times per day. Each possibility represents a separate embodiment of the invention.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g., decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21st Ed., Lippincott Williams & Wilkins, Philadelphia, Pa., (2005).

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, NY (2017)].

In some embodiments, the effective amount or dose of the active ingredient can be estimated initially from in vitro assays. In one embodiment, a dose can be formulated in animal models and such information can be used to more accurately determine useful doses in humans. In some embodiments, the effective amount or dose of the active ingredient can be estimated by performing a diagnostic method described herein (e.g., a detectable probe-based imaging).

The present disclosure also provides methods for treating or preventing cancer in a subject, comprising administering to the subject a therapeutically effective amount of the compound, the conjugate, or the composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject. Methods of killing a cancer or tumor cell are also provided comprising contacting the cancer or tumor cell with an effective amount of the compound, the conjugate, or the composition as described herein. In some embodiments, the compounds or the conjugates of the invention can inhibit the cathepsin activity (intra-, and/or extracellular activity). The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic composition prior to, during, or after surgical intervention to remove part or all of a tumor.

The term “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas. Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocelular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

### Methods of Administration

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intratumoral, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut,

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, c) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for case of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

### Methods of Production

In another aspect, there is provided a method of producing a conjugate, the method comprising: providing the precursor of the invention and a reactant having a reactivity to the precursor; and contacting the precursor with the reactant under suitable conditions, thereby obtaining a conjugate.

In some embodiments, the reactant has a reactivity to Z moiety of the precursor. In some embodiments, the reactant comprises a thio group, an amino group or both. In some embodiments, the reactant comprises a thio group. In some embodiments, a thio group is a thiol group. In some embodiments, the reactant is the targeting molecule. In some embodiments, the reactant is a macro-molecule having a reactivity to the precursor. In some embodiments, the reactant is an antibody or antigen binding fragment thereof. In some embodiments, the reactant is an antibody. In some embodiments, the reactant is an antibody or antigen binding fragment thereof comprising at least one reduced cysteine. In some embodiments, at least 50%, at least 90%, or at least 99% of the cysteines within the antibody or antigen binding fragment thereof are reduced. Each possibility represents a separate embodiment of the invention.

In some embodiments, contacting is performed at a molar ratio between the reactant and the precursor between about 1:2 and between about 1:300, between about 1:3 and 1:200, between about 1:10 and 1:200, between about 1:30 and 1:200, between about 1:50 and about 1:200, including any range in between, wherein the reactant is a macro-molecule having a reactivity to the precursor. In some embodiments, the reactant is an antibody or an antigen binding fragment thereof, wherein contacting is performed at a molar ratio between the antibody or an antigen binding fragment thereof and the precursor between about 1:2 and between about 1:300, between about 1:3 and 1:200, between about 1:10 and 1:200, between about 1:30 and 1:200, between about 1:50 and about 1:200, including any range in between.

In some embodiments, the method further comprises selecting the antibody or antigen binding fragment thereof. In some embodiments, the selecting comprises selecting an antibody that binds to a surface molecule expressed on a surface of a target cell. In some embodiments, the antibody binds to an antigen present on the surface of a target cell. In some embodiments, the molecule is an antigen. In some embodiments, the antigen is a protein. In some embodiments, the antigen is a peptide. In some embodiments, the peptide is a peptide of an infectious agent. In some embodiments, an infectious agent is a pathogen. In some embodiments, the infectious agent is a virus. In some embodiments, the infectious agent is a bacterium. In some embodiments, the virus is SARS-Cov-2. In some embodiments, the protein is a receptor. In some embodiments, the receptor is specific to the target cell. In some embodiments, the receptor characterizes the target cell. In some embodiments, the antigen is a cancer specific antigen. In some embodiments, a cancer specific antigen is a tumor specific antigen. Antibodies that bind to infectious agent peptides, tumor specific antigens and tissue/cell type specific receptors are well known in the art. Any such antibody can be used as the T-moiety.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

In some embodiments, the target cell is a disease cell. In some embodiments, the target cell is a cancerous cell. In some embodiments, the target cell is an infected cell. In some embodiments, the target cell is of a tissue or cell type of the disease. In some embodiments, the target cell is treatable by cathepsin inhibition. In some embodiments, the target cell is treatable by a method of the invention. In some embodiments, the target cell is infected by a pathogen and the antibody binds a pathogen protein or peptide. In some embodiments, the target cell is infected by a pathogen and the antibody binds a receptor expressed on the surface of cells infected by the pathogen. In some embodiments, the receptor is a virus binding receptor. In some embodiments, the receptor is angiotensin-converting enzyme 2 (ACE2). In some embodiments, the virus is a coronavirus and the virus binding receptor is angiotensin-converting enzyme 2 (ACE2). In some embodiments, the target cell is a cancer cell and the antibody binds a cancer antigen. In some embodiments, a cancer antigen is a cancer specific antigen. In some embodiments, the target cell is a neuronal cell and the antibody binds a neuronal protein. In some embodiments, a neuronal protein is a neuronal receptor. In some embodiments, a neuronal protein is a neuronal marker. In some embodiments, the target cell is an immune cell and the antibody binds to an immune cell protein. In some embodiments, an immune cell protein is an immune receptor. In some embodiments, an immune cell protein is an immune marker. In some embodiments, an immune cell protein is a protein target of an immunotherapy. In some embodiments, an immune cell protein is an immune checkpoint protein. In some embodiments, the antibody binds to an immune checkpoint protein or its ligand. In some embodiments, the antibody is an anti-PDL1 antibody.

In some embodiments, the method further comprises confirming the conjugate binds to a target cell. In some embodiments, the method further comprises testing binding of the conjugate to a target cell. In some embodiments, the method further comprises selecting a conjugate that binds to a target cell. In some embodiments, a conjugate that binds at a comparable level to the antibody when not conjugated is selected. In some embodiments, comparable is with a variance of less than 10%. In some embodiments, confirming or testing binding is by a method as provided hereinbelow. Generally, methods of determining binding of an antibody or antibody drug conjugate (ADC) to a target protein or cell are well known and any method of such testing may be performed. In some embodiments, ADC represents an exemplary conjugate of the invention.

In some embodiments, the method further comprises confirming delivery of the cathepsin inhibitor to the interior of a target cell. In some embodiments, the method further comprises testing delivery of the cathepsin inhibitor to the interior of a target cell. In some embodiments, the delivery is upon binding to the target cell. In some embodiments, a target cell is a cell to which the conjugate has bound. In some embodiments, the interior is a lysosome. In some embodiments, the interior is the cytoplasm. In some embodiments, the interior is an endosome. In some embodiments, the confirming or testing is confirming or testing endocytosis of the receptor upon binding of the conjugate. In some embodiments, the method further comprises selecting a conjugate that is delivered to the interior of a target cell upon binding to the target cell.

In some embodiments, the method further comprises confirming reduction of cathepsin activity in the target cell. In some embodiments, the method further comprises testing cathepsin activity in the target cell. In some embodiments, the reduction is after the conjugate binds. In some embodiments, the method further comprises selecting a conjugate that causes reduction of cathepsin activity in a target cell.

In some embodiments, the method further comprises confirming treatment of a disease. In some embodiments, the method further comprises testing the ability of the conjugate to treat disease. In some embodiments, treatment is upon contact of the conjugate with a disease cell. In some embodiments, contact comprises administration of the conjugate. In some embodiments, the method further comprises selecting a conjugate that treats the disease. In some embodiments, the disease is cancer. In some embodiments, the method further comprises confirming the conjugate improves an immunotherapy treatment. In some embodiments, the method further comprises testing the effect of the conjugate on an immunotherapy treatment. In some embodiments, the confirming or testing is in a cancer with cathepsin activity levels above a predetermined threshold. In some embodiments, the confirming or testing is in a sample or subject selected by a method of the invention. In some embodiments, the method further comprises selecting a conjugate that improves an immunotherapy treatment.

### Methods of Predicting Response to Immunotherapy

By another aspect, there is provided a method for predicting response of a subject to an immunotherapy treatment, the method comprising:
determining a level of cathepsin in the subject or in a sample obtained from the subject, wherein a level above a predetermined threshold indicates the subject is unlikely to respond to the immunotherapy;
thereby predicting response of a subject to an immunotherapy treatment.

In some embodiments, the method is a diagnostic method. In some embodiments, the method is an in vitro method. In some embodiments, the method is an ex vivo method. In some embodiments, the method is a computer implemented method. In some embodiments, the method is a statistical method. In some embodiments, the method is a method that cannot be performed in a human mind.

In some embodiments, the method is for predicting response to treatment. In some embodiments, the method is for determining response to treatment. In some embodiments, predicting is determining. In some embodiments, treatment is immunotherapy. In some embodiments, treatment is immunotherapy treatment. In some embodiments, predicting is predicting the probability of response. In some embodiments, response is being likely to respond. In some embodiments, response is being unlikely to respond. In some embodiments, the determining is determining if the subject is a responder to immunotherapy. In some embodiments, the determining is determining if the subject is a non-responder to immunotherapy. In some embodiments, a responder is a subject that responders. In some embodiments, a non-responder is a subject that does not respond. In some embodiments, determining is determining likelihood of response. In some embodiments, not responding is being unlikely to respond. In some embodiments, responding is being likely to respond. In some embodiments, being unlikely is being highly unlikely. In some embodiments, unlikely is less than a 1, 3, 5, 7, 10, 15, 20, 25, 30, 35, 40, 45 or 50% chance of responding. Each possibility represents a separate embodiment of the invention. In some embodiments, the method is for monitoring response to the therapy. In some embodiments, the method is for determining if the therapy should continue or be adjusted (e.g., by further treating the subject with an additional therapy or increasing the dose/frequency of the immunotherapy).

In some embodiments, non-response comprises progressive disease or disease progression. In some embodiments, non-response comprises cancer progression. In some embodiments, non-response comprises stable disease. In some embodiments, non-response comprises a worsening of symptoms of the disease. In some embodiments, non-response is not the development of side effects. In some embodiments, non-response comprises growth, metastasis and/or continued proliferation of a cancer. In some embodiments, response is stable disease. In some embodiments, response comprises remission. In some embodiments, remission is minimal remission. In some embodiments, remission is partial remission. In some embodiments, remission is complete remission. A trained physician will be familiar with methods of determining response and such method may be employed.

As used herein, the term “responder” or a subject “known to respond” are used interchangeably and refer to a subject that when administered a treatment displays an improvement in at least one criteria of the disease being treated by the treatment or does not show an increase in severity of the disease. In some embodiments, a responder is a subject that when administered a treatment displays an improvement in the disease that is being treated by the treatment. In some embodiments, a responder is a subject that when administered a treatment does not show an increase in severity of the disease. In some embodiments, an increase is severity is over time. In some embodiments, does not show an increase in severity is stable disease. In some embodiments, a responder is a subject for which the treatment produces an anti-disease response. In some embodiments, for a subject with cancer, a responder is a subject in which the treatment produces an anticancer response. In some embodiments, a response is not a reduction in side effects. In some embodiments, a response is a reduction in side effects. In some embodiments, a response is a response against the disease itself. In some embodiments, an anticancer response is an antitumor response. In some embodiments, an antitumor response comprises tumor regression. In some embodiments, an antitumor response comprises tumor shrinkage. In some embodiments, an antitumor response comprises a lack of tumor growth. In some embodiments, an antitumor response comprises a lack of tumor metastasis. In some embodiments, an antitumor response comprises a lack of tumor hyperproliferation. In some embodiments, an improvement is in at least one symptom of the disease. In some embodiments, response is complete response. In some embodiments, response is minimal response. In some embodiments, response is partial response. In some embodiments, response comprises stable disease. In some embodiments, responder is a subject with a favorable response to the therapy. In some embodiments, non-responder is a subject with a non-favorable response to the therapy. In some embodiments, a non-favorable response is an increase in tumor burden. Increases in tumor burden can encompass any increase in tumor size or total cancer cell number such as increase in tumor size, increase in tumor spread, increase in metastasis, increase in tumor cell proliferation or any other increase.

As used herein, a “favorable response” of the cancer patient indicates “responsiveness” of the cancer patient to the treatment with the treatment, namely, the treatment of the responsive cancer patient with the treatment will lead to the desired clinical outcome such as tumor regression, tumor shrinkage or tumor necrosis; reduction in tumor burden; an anti-tumor response by the immune system; preventing or delaying tumor recurrence, tumor growth or tumor metastasis. In some embodiments, a subject that is not a non-responder is a responder.

As used herein, the term “non-responder” and a subject “known to not respond” are used interchangeably and refer to a subject that when administered a treatment displays no improvement or stabilization in disease. In some embodiments, a non-responder displays a worsening of disease when administered a treatment. In some embodiments, non-responder is not a subject that experiences a side effect of the therapy. In some embodiments, a non-responder is a subject in which the disease progresses. In some embodiments, a non-responder is a subject in which the disease does not stabilize after treatment. In some embodiments, a non-responder is a subject in which the disease does not improve after treatment. In some embodiments, a non-responder is a subject that is not a responder as defined hereinabove. In some embodiments, a non-responder is a subject with a non-favorable response to the therapy. In some embodiments, a non-responder is a subject resistant to the therapy. In some embodiments, a non-responder is a subject refractory to the therapy.

As used herein a “non-favorable response” of the cancer patient indicates “non-responsiveness” of the cancer patient to the treatment with the treatment and thus the treatment of the non-responsive cancer patient with the treatment will not lead to the desired clinical outcome, and potentially to a non-desired outcomes such as tumor expansion, recurrence, or metastases. In some embodiments, the method further comprises discontinuing administration of the treatment to a subject that is a non-responder. In some embodiments the method further comprises continuing to administer the treatment to a subject, in combination with an additional therapy. In some embodiments, the additional therapy increases responsiveness of a non-responsive patient.

In some embodiments, the subject is a mammal. In some embodiments, the subject is a human. In some embodiments, the subject suffers from a disease. In some embodiments, the disease is treatable by the treatment. In some embodiments, the disease is cancer. In some embodiments, the disease is treatable by an immune checkpoint inhibitor (ICI). In some embodiments, the cancer is a PD-L1 positive cancer. In some embodiments, the cancer is a PD-L1 negative cancer. In some embodiments, the cancer is solid cancer. In some embodiments, the cancer is a tumor. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is selected from skin cancer, and lung cancer. In some embodiments, the cancer is skin cancer. In some embodiments, the cancer is lung cancer. In some embodiments, the skin cancer is melanoma. In some embodiments, the subject is naïve to treatment before the determining. In some embodiments, the subject has not received the treatment before the determining. In some embodiments, the subject has received the treatment previously. In some embodiments, the subject is naïve to any treatment. In some embodiments, the subject is naïve to immunotherapy. In some embodiments, the treatment is the first line of treatment. In some embodiments, the treatment is an advanced line of treatment.

Typically, the subject is afflicted with cancer that is of the type that responds to immunotherapy and in particulate immune checkpoint inhibition. Non-Limiting examples of such cancers being: melanoma, lung cancer, breast cancer, bladder cancer, cervical cancer, colon cancer, head and neck cancer, or Hodgkin lymphoma. Immunotherapy has been approved for the treatment of the following cancers: Bladder cancer, Breast cancer, Cervical cancer, Colorectal cancer, Esophageal cancer, Head and neck cancer, Kidney cancer, Leukemia, Liver cancer, Lung cancer, Lymphoma, Melanoma, Prostate cancer, and Skin cancer. Most preferably the cancer is melanoma.

As used herein, the term “anti-cancer immunotherapy” includes strategies used to activate effector immune cells to increase the efficacy of the patient's own immune response against neoplastic cells (e.g., lymphoma, melanoma, lung carcinoma, glioblastoma, renal carcinoma, gastrointestinal stromal carcinoma and leukemia). Immunotherapy includes cancer vaccine, oncolytic viruses, cell therapies and antibody conjugated drugs. By a preferred embodiment the anti-cancer therapy is immune checkpoint inhibition therapy.

In some embodiments, the treatment is an anticancer treatment. In some embodiments, the treatment is immunotherapy. In some embodiments, the anticancer treatment is immunotherapy. In some embodiments, the immunotherapy is selected from immune checkpoint inhibition, immune checkpoint modulation, immune checkpoint blockade, adoptive-cell transfer therapy, oncolytic virus therapy, vaccine therapy, immune system modulation and therapy using monoclonal antibodies. In some embodiments, an immunotherapy is selected from immune checkpoint inhibitors, immune checkpoint modulators, and immune checkpoint blockers. In some embodiments, the immunotherapy is an immune checkpoint inhibitor. In some embodiments, the immunotherapy is immune checkpoint blockade. In some embodiments, an immunotherapy is administered in combination with one or more conventional cancer therapy including chemotherapy, targeted therapy, steroids, and radiotherapy. Combinations of ICI and chemotherapy/radiotherapy/targeted therapy have been studied in multiple clinical trials. It will be understood by a skilled artisan that the predictive method disclosed herein is predictive in immunotherapy as a monotherapy, as well as part of a combination therapy.

In some embodiments, the immunotherapy is a plurality of immunotherapies. In some embodiments, the immunotherapy is immune checkpoint blockade. In some embodiments, the immunotherapy is immune checkpoint protein inhibition. In some embodiments, the immunotherapy is immune checkpoint protein modulation. In some embodiments, the immunotherapy comprises immune checkpoint inhibition. In some embodiments, the immunotherapy comprises immune checkpoint modulation. In some embodiments, immune checkpoint blockade and/or immune checkpoint inhibition comprises administering to the subject an immune checkpoint inhibitor. In some embodiments, inhibition comprises administering an immune checkpoint inhibitor. In some embodiments, the inhibitor is a blocking antibody. In some embodiments, the immunotherapy comprises immune checkpoint blockade. In some embodiments, modulation comprises administering an immune checkpoint modulator. In some embodiments, immune checkpoint modulation comprises administering to the subject an immune checkpoint modulator.

As used herein, the term “an immune checkpoint inhibitor (ICI)” refers to a single ICI, a combination of ICIs and a combination of an ICI with another cancer therapy. The ICI may be a monoclonal antibody, a dual-specific antibody, a humanized antibody, a fully human antibody, a fusion protein, or a combination thereof directed to blocking, inhibition or modulation of immune checkpoint proteins. In some embodiments, an immune checkpoint inhibitor is an immune checkpoint modulator. In some embodiments, an immune checkpoint inhibitor is an immune checkpoint blocker. In some embodiments, the immune checkpoint protein is selected from PD-1 (Programmed Death-1); PD-L1; PD-L2; CTLA-4 (Cytotoxic T-Lymphocyte-Associated protein 4); A2AR (Adenosine A2A receptor), also known as ADORA2A; B7-H3, also called CD276; B7-H4, also called VTCN1; B7-H5; BTLA (B and T Lymphocyte Attenuator), also called CD272; IDO (Indoleamine 2,3-dioxygenase); KIR (Killer-cell Immunoglobulin-like Receptor); LAG-3 (Lymphocyte Activation Gene-3); TDO (Tryptophan 2,3-dioxygenase); TIM-3 (T-cell Immunoglobulin domain and Mucin domain 3); VISTA (V-domain Ig suppressor of T cell activation); NOX2 (nicotinamide adenine dinucleotide phosphate NADPH oxidase isoform 2); SIGLEC7 (Sialic acid-binding immunoglobulin-type lectin 7), also called CD328; SIGLEC9 (Sialic acid-binding immunoglobulin-type lectin 9), also called CD329; OX40 (Tumor necrosis factor receptor superfamily, member 4) also called CD134; and TIGIT. In some embodiments, the immune checkpoint protein is selected from PD-1, PD-L1 and PD-L2. In some embodiments, the immune checkpoint protein is selected from PD-1 and PD-L1. In some embodiments, the immune checkpoint protein is CTLA-4. In some embodiments, the immune checkpoint protein is PD-1. In some embodiments, immune checkpoint blockade comprises an anti-PD-1/PD-L1/PD-L2 immunotherapy. In some embodiments, immune checkpoint blockade comprises an anti-PD-1 immunotherapy. In some embodiments, immune checkpoint blockade comprises an anti-PD-1 and/or anti-PD-L1 immunotherapy. In some embodiments, immune checkpoint blockade comprises an anti CTLA-4 immunotherapy. In some embodiments, immune checkpoint blockade comprises an anti-PD-1 and/or anti-PD-L1 immunotherapy and an anti CTLA-4 immunotherapy. In some embodiments, the immunotherapy comprises administering the ICI.

Non-limiting examples of immune checkpoint inhibitors include but are not limited to those provided in Table 1.

TABLE 1
Clinically approved ICIS

| | Brand | | | |
|---|---|---|---|---|
| Name | Name | Target | Approved | Indications |
| Ipilimumab | Yervoy | CTLA-4 | 2011 | metastatic melanoma, renal cell |
| | | | | carcinoma, colorectal |
| | | | | cancer, hepatocellular |
| | | | | carcinoma, non-small cell lung |
| | | | | cancer, malignant pleural |
| | | | | mesothelioma |
| Nivolumab | Opdivo | PD-1 | 2014 | metastatic melanoma, non-small |
| | | | | cell lung cancer, renal cell |
| | | | | carcinoma, Hodgkin's |
| | | | | lymphoma, head and neck |
| | | | | cancer, urothelial carcinoma, |
| | | | | colorectal cancer, hepatocellular |
| | | | | carcinoma, small cell lung |
| | | | | cancer, esophageal carcinoma, |
| | | | | malignant pleural mesothelioma |
| Pembrolizumab | Keytruda | PD-1 | 2014 | metastatic melanoma, non-small |
| | | | | cell lung cancer, head and neck |
| | | | | cancer, Hodgkin's lymphoma, |
| | | | | urothelial carcinoma, gastric |
| | | | | cancer, cervical cancer, |
| | | | | hepatocellular carcinoma, Merkel |
| | | | | cell carcinoma, renal cell carcinoma, |
| | | | | small cell lung cancer, esophageal |
| | | | | carcinoma, endometrial |
| | | | | cancer, squamous cell carcinoma |
| Atezolizumab | Tecentriq | PD-L1 | 2016 | bladder cancer, non-small cell lung |
| | | | | cancer, breast cancer, small cell |
| | | | | lung cancer, hepatocellular |
| | | | | carcinoma, metastatic melanoma |
| Avelumab | Bavencio | PD-L1 | 2017 | Merkel cell carcinoma, urothelial |
| | | | | carcinoma, renal cell carcinoma |
| Durvalumab | Imfinzi | PD-L1 | 2017 | non-small cell lung cancer, small |
| | | | | cell lung cancer |
| Cemiplimab | Libtayo | PD-1 | 2018 | squamous cell carcinoma, basal cell |
| | | | | carcinoma, non-small cell lung |
| | | | | cancer |

The cathepsin of the invention are any type of cathepsin but in accordance with the present invention they are preferably cysteine proteases and may be one or more of the following cathepsin: B, C, F, H, K, L, O, S, V, X and W. Preferably the cathepsin is one of B, L or S or a combination of at least two (preferably B and S) or all three of them. In some embodiments, the cathepsin is selected from cathepsin B, C, F, H, K, L, O, S, V, X and W. In some embodiments, cathepsin is a cysteine cathepsin. In some embodiments, the cathepsin is cathepsin B, C, F, H, K, L, O, S, V, X or W. Each possibility represents a separate embodiment of the invention. In some embodiments, the cathepsin is cathepsin B. In some embodiments, the cathepsin is cathepsin L. In some embodiments, the cathepsin is cathepsin S. In some embodiments, the cathepsin is a combination of at least two of cathepsin B, L and S. In some embodiments, the cathepsin is all of cathepsin B, cathepsin L and cathepsin S.

The cathepsin level may refer to the level of the cathepsin expression product such as the mRNA level (by blots, probes and amplification techniques or the protein level (by western blot, by antibodies (Eliza) etc. In some embodiments, cathepsin level is cathepsin protein level. In some embodiments, cathepsin level is cathepsin mRNA level. In some embodiments, cathepsin level is cathepsin expression level.

By another option the level of the cathepsin enzymatic activity is measured. In some embodiments, the cathepsin level is cathepsin activity level. The measurement may be carried out by measuring the enzymatic transformation of Cathepsin substrates (peptides of 2-10 amino acids) into products (peptides of 1-9 amino acids) for example by a FRET assay or mass spectrometry or in cell-based assays or quenched substrates or by fluorogenic substrates. Examples are provided in reviews by Galia Blum, 2008, “Use of fluorescent imaging to investigate pathological protease activity. Curr. Opi. Drug Discov. & Develop., 11 (5): 708-16 and Edgington et al., “Functional imaging of proteases: recent advances in the design and application of substrate-based and activity-based probes”, Current Opinion in Chemical Biology 2011, 15:798-805 all of which are incorporated herein by reference in their entirety. Inhibition of Cathepsin can be also assessed at the functional level for example by testing the inhibitory effect by measuring changes of antigen repertoire, T cell activation and accumulation of CD74 (p10) protein. Another option is the use of cathepsin activity-based probes that work in vitro, ex vivo or in vivo. In some embodiments, the determining is determining the activity level of a cathepsin. In some embodiments, the determining is determining the activity level of a cathepsin selected from B, L and S. In some embodiments, the determining is determining the activity level of all of cathepsin B, L and S.

In some embodiments, the determining is in macrophages. In some embodiments, the determining is determining the level of cathepsin in macrophages. In some embodiments, the macrophages are M2 macrophages. In some embodiments, M2 macrophages are MΦ. In some embodiments, the macrophages are inhibitory macrophages. Methods of identifying macrophages and M2 macrophages are well known in the art and are also provided herein. Any such method may be employed. For example, macrophages can be identified as being CD45 positive and CD68 positive and also CD3 negative. M2 macrophages can further be identified as being CD163 positive. It will be understood however, that any macrophage markers can be used. In some embodiments, the macrophages are tumor associated macrophages (TAMs).

In some embodiments, the determining is carried out in the subject. In some embodiments, the determining is in vivo determining. In some embodiments, the determining is carried out in the tumor. In some embodiments, the determining is carrier out in the tumor and/or tumor microenvironment (TME). In some embodiments, the determining is carried out in tumor associated or resident macrophages. In some embodiments, the determining is carried out in a sample. In some embodiments, the sample comprises macrophages.

In some embodiments, the sample is obtained from the subject. In some embodiments, the method further comprises obtaining a sample from the subject. In some embodiments, the method further comprises receiving a sample obtained from the subject. In some embodiments, the sample is a cancer sample. In some embodiments, the sample is a cancer sample. In some embodiments, the sample is a tumor sample. In some embodiments, the sample is a biopsy. In some embodiments, the sample comprises tumor infiltrating immune cells. In some embodiments, the immune cells are lymphocytes (TILs). In some embodiments, the immune cells are TAMs. In some embodiments, the sample is a bodily fluid sample. In some embodiments, the fluid is selected from blood, plasma, cerebrospinal fluid, urine, and sperm. In some embodiments, the bodily fluid is selected from, blood, plasma, serum, lymph, cerebral spinal fluid, urine, feces, semen, tumor fluid, milk, vitreous fluid and gastric fluid.

In some embodiments, the number of macrophages with high cathepsin levels is measured. In some embodiments, measured is determined. In some embodiments, the percentage of macrophages with high cathepsin levels is measured. In some embodiments, cathepsin levels is activity levels. In some embodiments, percentage is percentage of all macrophages in the sample. In some embodiments, percentage is percentage of all macrophages in the tumor. In some embodiments, high is above a predetermined threshold. In some embodiments, above is at least 5, 10, 15, 20, 25, 30, 35, 40, 45 50, 55, 60, 65, 70, 75, 80, 85, 90, 95 or 100% above. Each possibility represents a separate embodiment of the invention. In some embodiments, the predetermined threshold is the cathepsin levels in control macrophages. In some embodiments, control macrophages are macrophages that are not in the tumor. In some embodiments, the control macrophages are macrophages from the subject but not in the tumor. In some embodiments, the control macrophages are macrophages from subjects that respond to the immunotherapy. In some embodiments, the control macrophages are from responders. In some embodiments, the control macrophages are from tumors that respond to the immunotherapy. In some embodiments, the control macrophages are from tumors from responders.

In some embodiments, a cathepsin level above a predetermined threshold level indicates the subject is unlikely to respond. In some embodiments, a cathepsin level above a predetermined threshold level indicates the subject is a non-responder. In some embodiments, a cathepsin activity level above the predetermined threshold is indicative. In some embodiments, a cathepsin activity level in macrophages above the predetermined threshold is indicative. In some embodiments, a number of macrophages with a high cathepsin activity level above the predetermined threshold is indicative. In some embodiments, a percentage of macrophages with a high cathepsin activity level above the predetermined threshold is indicative.

In some embodiments, the predetermined threshold is the cathepsin level in a control subject. In some embodiments, a control subject is a plurality of control subjects. In some embodiments, a control subject is a population of control subjects. In some embodiments, control subjects are subjects determined to respond to the treatment. In some embodiments, the control subject are subjects with the same cancer as the subject and determined to respond. In some embodiments, the control subjects are responders. In some embodiments, in a control subject is in a sample from the control subject. In some embodiments, in a control subject is in macrophages from the control subject. In some embodiments, the in a control subject is in macrophages in a sample from the control subject. In some embodiments, the same cancer is the same type of cancer. In some embodiments, the same cancer is cancer of the same tissue or cell type.

By one option the method of the invention is carried on an immunotherapy naïve subject—i.e., a subject that has not yet begun immunotherapy treatment. Although the subject may have been treated by other anti-cancer treatments, preferably the subject has not been treated by any anti-cancer therapy. In such a case the cathepsin level or activity in the sample is compared to a threshold prepared by measuring the cathepsin level/activity in a plurality of clinically established responsive vs. clinically established nonresponsive subjects having the same cancer and are about to be treated by the same intended anti-cancer immunotherapy drug. If the specific subject's level of cathepsin or level of cathepsin activity is different than the respondents in a significant manner (preferably a higher level than that of the respondents typically closer to the high level of the non-respondent) that subject has a high likelihood of not responding to the therapy.

In some embodiments, above a predetermined threshold is the same as levels in a non-responding subject. In some embodiments, a non-responding subject is a plurality of non-responding subjects. In some embodiments, a non-responding subject is a population of non-responding subjects. In some embodiments, a level in a plurality of subjects or a population is the average level. In some embodiments, a level in a plurality of subjects or a population is the mean level. In some embodiments, a level in a plurality of subjects or a population is the maximum level. In some embodiments, a level in a plurality of subjects or a population is the minimum level. In some embodiments, a level in a plurality of subjects or a population is the 75% percentile value. In some embodiments, a level in a plurality of subjects or a population is the 90% percentile value. In some embodiments, the same as is within a 30, 25, 20, 15, 10, 5 or 1% variance. Each possibility represents a separate embodiment of the invention.

By another option the method includes measuring the cathepsin level or activity before the contact with the anti-cancer immunotherapy treatment and doing the same measurement after a single contact, or after a few contact cycles. If the cathepsin level increases—this means that the subject has a high likelihood of not responding to the anticancer immunotherapy treatment (as compared to respondents for which the level of the expression product or the activity either stays the same or decreases).

In some embodiments, the determining comprises determining cathepsin level at a first time point. In some embodiments, the determining comprises determining cathepsin level at a second time point. In some embodiments, at least one contact with the treatment occurs between the first and second time points. In some embodiments, the first time point is before contact with the treatment. In some embodiments, contacting is in vivo contacting. In some embodiments, in vivo contacting comprises administering the treatment. In some embodiments, the contacting is in vitro contacting. In some embodiments, in vitro is in culture. In some embodiments, the sample is contacted. In some embodiments, cells from the sample are contacted. In some embodiments, the cells are immune cells. In some embodiments, the cells are a mix of cancer cells and immune cells. In some embodiments, the immune cells are tumor resident immune cells. In some embodiments, the immune cells are macrophages. In some embodiments, cathepsin levels are determined in a portion of the cells from the sample not contacted with the treatment (i.e., the first time point) and in a portion of cells from the ample contacted with the treatment (i.e., the second time point). In some embodiments, a first sample is obtained at the first time point and a second sample is obtained at the second time point.

In some embodiments, an increase in cathepsin level from the first time point to the second time point indicates the subject is unlikely to respond. In some embodiments, an increase in cathepsin level from the first time point to the second time point indicates the subject is a non-responder. In some embodiments, an increase in cathepsin level between the first and the second time point indicates the subject is unlikely to respond. In some embodiments, an increase in cathepsin level between the first and the second time point indicates the subject is a non-responder. In some embodiments, an increase is a significant increase. In some embodiments, significant is statistically significant. In some embodiments, an increase is at least a 5, 10, 15, 20, 25, 30, 35, 40, 45, 50, 55, 60, 65, 70, 75, 80, 85, 90, 95 or 100% increase. Each possibility represents a separate embodiment of the invention. In some embodiments, an increase is at least a 20% increase. In some embodiments, cathepsin level is cathepsin activity level. In some embodiments, cathepsin level is cathepsin activity level in macrophages. In some embodiments, cathepsin level is the number or percentage of macrophages with high cathepsin activity levels.

The contact may be in vivo—i.e., administration to the subject of one cycle or a few cycles of the anti-cancer immunotherapy drug (the determination of the cathepsin level or activity may be ex vivo or in vivo). The contact may also be ex vivo—i.e., first measuring the level or activity in a sample (cancer biopsy), then contacting the sample with the drug ex vivo (one or several times) and then measuring the level or activity again. As indicated above the measurement may be done either in an ex vivo sample obtained from the subject or in vivo in the body of the subject.

The sample may be a body fluid sample (blood, plasma, cerebrospinal fluid, urine, sperm, etc.) but preferably the sample is a biopsy from the cancer. The sample can be lysed to release its intercellular contents to the test medium. In some embodiments, the sample is lysed. In some embodiments, the sample is not lysed. The level of the expression product may be measured by standard methods for measuring mRNA or protein levels. The level of the cathepsin activity may be carried out by using activity-based probes that produce a detectable label-fluorescent, chemi/bioluminescent, radio label, colorimetric reaction or other means of detection MRI, (we have X ray and are making ultrasound reagents)/in the presence of cathepsin activity. In some embodiments, the determining is with a cathepsin activity-based probe. In some embodiments, the determining comprises contacting the sample with a cathepsin activity-based probe. In some embodiments, the determining comprises administering to the subject a cathepsin activity-based probe.

As used herein, the term “cathepsin activity-based probe” (also used herein as “the probe”) refers to a molecule that measures cathepsin activity. In some embodiments, the probe is or comprises a 2-10 amino acid residues long peptide. In some embodiments, the probe is an irreversible cathepsin inhibitor. In some embodiments, a probe outputs a detectable signal proportional to the cathepsin activity. In some embodiments, the probe is a cathepsin B, L or S-activity based probe. In some embodiments, the probe is a cathepsin B, L and S-activity based probe. Non-limiting examples of such probes are defined in Galia, 2008, “Use of fluorescent imaging to investigate pathological protease activity”, Curr. Opi. Drug Discov. & Develop., 11 (5): 708-16 and Edgington et al., “Functional imaging of proteases: recent advances in the design and application of substrate-based and activity-based probes”, Current Opinion in Chemical Biology 2011, 15:798-805, and Schleyer and Cui, “Molecular probes for selective detection of cysteine cathepsins”, Org. Biomol. Chem., 2021, 19, 6182, the contents of which are all hereby incorporated herein by reference in their entirety.

In some embodiments, the probe is represented by Formula 4A:

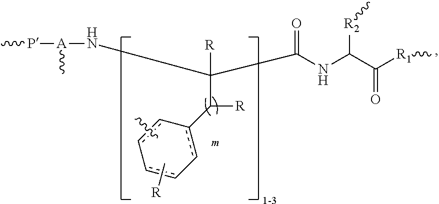

wherein: P′ is an amine protecting group; A is a bond or an amino acid residue; R1 is or comprises —NH—R′ (wherein R′ is as disclosed hereinabove), chloromethyl ketone, acyloxymethyl ketone, a Michael acceptor, phosphonate, cyano group, or

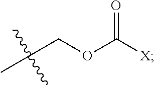

wherein X is selected from a substituted or unsubstituted alkyl; a substituted or unsubstituted alkenyl, a substituted or unsubstituted aryl, a substituted or unsubstituted heteroaryl; each R is independently H, or represents at least one substituent; A represents at least one amino acid residue; m is an integer ranging between 1 and 5 (e.g. 1, 2, 3, 4, or 5 including any range between); n is an integer ranging between 1 and 3 (e.g. 1, 2 or 3 including any range between); R2 comprises a substituted or unsubstituted alkylamine, or —[C(D′)2]n-, wherein each D′ is independently H, an amino acid side chain, a substituent, an optionally substituted C1-C20 alkylamine or an optionally substituted C1-C20 alkylguanidine; a wavy bond is absent or represents an attachment point to H, or to an imaging moiety, wherein at least one wavy bond is the attachment point to the imaging moiety, wherein the imaging moiety is described hereinbelow.

In some embodiments, R1 is

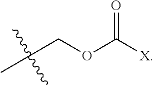

In some embodiments, R1 is

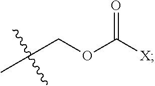

wherein X is a substituted aryl substituted aryl (including 2-nitro, 3-hydroxy benzyl and N-benzyloxycarbonyl [cbz]). In some embodiments, R1 is

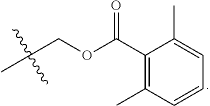 [SMILES: *COC(=O)c1c(C)cccc1C ; "."]

In some embodiments, R2 is —[C(D′)2]n-, wherein at least one D′ is a side chain of lysine, arginine or ornithine.

In some embodiments, the probe is represented by Formula 4:

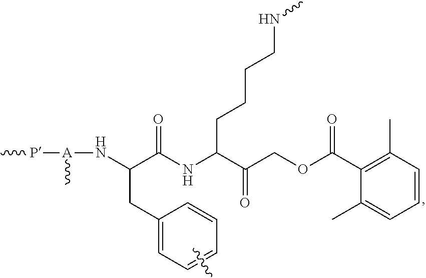

wherein P′ and A are as disclosed above.

In some embodiments, A is one or more aromatic amino acid residue and/or alanine residue. In some embodiments, A consists of one or more aromatic amino acid residue(s) selected from Phe, Tyr, His and Trp, including any non-natural or modified amino acid residues. In some embodiments, A is an alkyl-aryl, or an alkyl-carbocyclyl. In some embodiments, A comprises one or more Phe residue(s).

In some embodiments, amine protecting group is a carbamate-based protecting group. In some embodiments, amine protecting group is selected from 9-fluorenylmethyloxycarbonyl (Fmoc), Alloc, Dde, iv-Dde, benzyl, carboxy benzyl (Cbz), tert-butyloxycarbonyl (Boc), 2-[biphenylyl-(4)]-propyl-2-oxycarbonyl, dimethyl-3,5dimethoxybenzyloxycarbonyl, 2-(4-Nitrophenylsulfonyl) ethoxycarbonyl, 1,1-Dioxobenzo[b]thiophene-2-ylmethyloxycarbonyl, 2,7-Di-tert-butyl-Fmoc, 2-Fluoro-Fmoc, Nitrobenzenesulfonyl, Benzothiazole-2-sulfonyl, 2,2,2-Trichloroethyloxycarbonyl, and Dithiasuccinoyl, p-Nitrobenzyloxycarbonyl. In some embodiments, the amine protecting group is Cbz.

In some embodiments, the probe is represented by Formula 5:

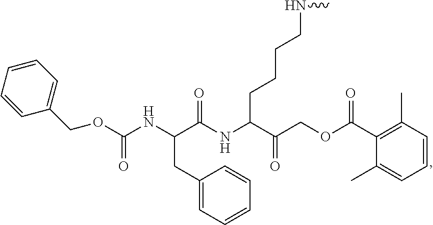

wherein the wavy bond represents the attachment point to the imaging moiety or to H. In some embodiments, the chemical moiety represented by any one of Formulae 4, 4A or 5 is bound to the imaging moiety via a linker. In some embodiments, the linker is any spacer, any polymeric linker (e.g. a polyether or a derivative thereof, a polyacrylate, a polyanhydride, a polyvinyl alcohol, a polysaccharide, a poly(N-vinylpyrrolidone), a polyglycerol (PG), a poly(N-(2-hydroxypropyl) methacrylamide), a polyoxazoline, a poly(amino acid)-based hybrid, a recombinant polypeptide, or a combination thereof), or wherein the linker is represented by the L variable disclosed herein.

In some embodiments, the imaging moiety comprises a luminophore, a fluorophore, a CT contrast agent, an MRI probe, a radioisotope, a dye, and a colorimetric probe. In some embodiments, the fluorophore is as described hereinabove In some embodiments, a luminophore comprises a compound capable of emitting luminescence upon excitation. In some embodiments, a luminophore comprises a luminescent transition metal complex (such as ruthenium tris-2,2′-bipyridine), inorganic luminophore (such as zinc sulfide doped with rare earth metal ions, rare earth metal oxysulfides doped with other rare earth metal ions, yttrium oxide doped with rare earth metal ions, zinc orthosilicate doped with manganese ions, quantum dots, etc.), a bioluminophore (e.g. luciferin, oxyluciferin, BtOxyLH2, AminoSeOxyLH2, coumarylaminooxyluciferin).

In some embodiments, the MRI probe comprises a metal selected from a superparamagnetic metal, a diamagnetic metal, a paramagnetic metal, a ferromagnetic metal, or any combination thereof. In some embodiments, at least one paramagnetic metal is selected from Barium (Ba), Tantalum (Ta), Tungsten (W), Dysprosium (Dy), Platinium (Pt), Gadolinium (Gd), and Manganese (Mn).

In some embodiments at least one diamagnetic metal is selected from Bismuth (Bi), and Gold (Au).

In some embodiments, the MRI probe comprises a metal ion. In some embodiments, the metal ion is selected from, without being limited thereto, gadolinium, iron, and manganese. In some embodiments, the MRI probe further comprises an organic metal coordinating compound (chelator). In some embodiments, the chelator comprises at least one metal coordinating chemical group. In some embodiments, the metal coordinating chemical group is selected from, without being limited thereto, imidazole, carboxylate, phosphate, and phosphonate. In some embodiments, the metal chelator is selected from, without being limited thereto, desferrioxamine (DFOA), tetraazacyclododecane-1,4,7,10-tetraacetic acid or gadoteric acid (DOTA), diethylenetriamine penta-acetic acid (DTPA) and dipyridoxyl diphosphate (DPDP). In some embodiments, the chelator having a high binding affinity and a specific coordination geometry towards a specific metal ion. In some embodiments, the MRI probe comprises a specific chelator-metal ion pair. In some embodiments, the specific pair is selected from desferrioxamine (DFOA)-Fe, tetraazacyclododecane-1,4,7,10-tetraacetic acid-Gd, gadoteric acid (DOTA)-Gd, diethylenetriamine penta-acetic acid (DTPA)-Mn, and dipyridoxyl diphosphate (DPDP)—Mn. In some embodiments, the chelator-metal ion complex is in the form of a cage or a metal-organic framework (MOF).

In some embodiments, the MRI probe comprises a SPION particle, a lanthanide series metal (or a cation thereof complexed by a chelator). In some embodiments, the MRI probe is selected from T1-class and T2-class MRI contrast agents.

The phrase “MRI contrast agents” refers to a group of contrast media typically used to improve the visibility of internal body structures in magnetic resonance imaging.

The phrase “T1-class and T2-class MRI contrast agents” is used herein to denote that tissue can be characterized by two different relaxation times, typically referred to as T1 and T2. T1 (longitudinal relaxation time) is known to a skilled artisan as the time constant which determines the rate at which excited protons return to equilibrium. It is a measure of the time taken for spinning protons to realign with the external magnetic field. T2 (transverse relaxation time) is known to a skilled artisan as the time constant which determines the rate at which excited protons reach equilibrium or go out of phase with each other. It is a measure of the time taken for spinning protons to lose phase coherence among the nuclei spinning perpendicular to the main field.

In some embodiments, the radioisotope is a positron emitting isotope, such as C-11, F-18, Ga-68, Lu-177, Cu-64, etc.). In some embodiments, the radioisotope is bound to the linker. In some embodiments, the radioisotope is a metal cation (e.g., Ga-68, Lu-177, Cu-64) coordinatively bound to a chelator. In some embodiments, the radioisotope is C-11 or F-18 covalently bound to a small molecule (e.g., a biologically active molecule such as deoxyglucose, a peptide, or a linker).

In some embodiments, the probe is GB111. GB111 is disclosed in Blum et al., 2007, “Noninvasive optical imaging of cysteine protease activity using fluorescently quenched activity-based probes”, Nat Chem Biol., 3, Pp. 668-677 which is hereby incorporated by reference in its entirety. GB111 (also used herein as “GB-111-NH₂) is represented by the formula:

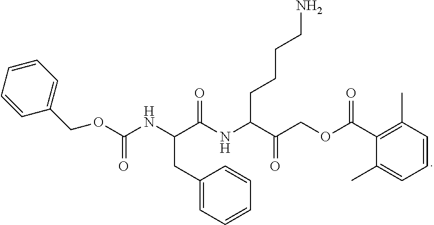 [SMILES: Cc1cccc(C)c1C(=O)OCC(=O)C(CCCCN)NC(=O)C(Cc1ccccc1)NC(=O)OCc1ccccc1 ; "."]

In some embodiments, GB111 is conjugated to a detectable imaging moiety (e.g., via the amino group of the lysine side chain). In some embodiments, the probe is configured to produce the detectable moiety in the presence of cathepsin activity. In some embodiments, the moiety is detectable in the presence of cathepsin activity. In some embodiments, the level of the moiety or level of detectability of the moiety is proportional to cathepsin activity. In some embodiments, the detectable moiety is a selected from a fluorescent moiety, a chemiluminescent moiety, a bioluminescent moiety, a radio-moiety, a dye, a colorimetric moiety and an imagine moiety. In some embodiments, the moiety is a fluorescent moiety. In some embodiments, a fluorescent moiety is a fluorophore. In some embodiments, the fluorophore is a flow cytometry detectable fluorophore. In some embodiments, the fluorophore is Cy5. In some embodiments, the fluorophore is Cy5, and the probe is GB123.

A preferred example of such an activity-based probe is GB123 also described in Blum et al 2007 and in U.S. Pat. No. 8,968,700, both of which are hereby incorporated herein by reference. In some embodiments, the probe is GB123. GB123 is represented by the formula:

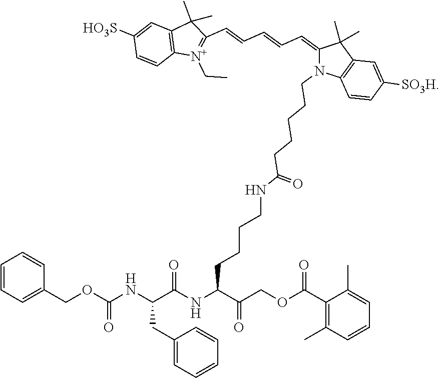 [SMILES: CC[N+]1=C(/C=C/C=C/C=C2\N(CCCCCC(=O)NCCCC[C@H](NC(=O)[C@H](Cc3ccccc3)NC(=O)OCc3ccccc3)C(=O)COC(=O)c3c(C)cccc3C)c3ccc(S(=O)(=O)O)cc3C2(C)C)C(C)(C)c2cc(S(=O)(=O)O)ccc21]

By another option the activity level is determined by using a substrate which produces a detectable label upon cleavage for example as described in Schleyer et al. In some embodiments, a detectable moiety is a detectable label. In some embodiments, the moiety is released upon cleavage. In some embodiments, the moiety is detectable upon cleavage. In some embodiments, cleavage is by the cathepsin.

By another option the determination of the cathepsin activity is carried out in vivo using a cathepsin activity probe attached to an imaging entity. In some embodiments, attached is conjugated to. In some embodiments, an imagine entity is an imaging moiety. In some embodiments, the moiety is an imagine moiety. In some embodiments, the imagine moiety is gold. In some embodiments, gold is a gold nanoparticle. An example of a probe for CT imaging is provided in Tsvirkun, et al., 2018, “CT imaging of enzymatic activity in cancer using covalent probes reveal a size-dependent pattern,” J Am Chem Soc. 2018 Sep. 26; 140 (38): 12010-12020, and International Patent Application WO2017141251 both of which are hereby incorporated herein by reference in their entirety. A non-limiting example of a probe for use in an in vivo assay is a cathepsin based probe complexed with/conjugated to a gold imaging agent, being a gold nanoparticle coated with polyethylene glycol attached to GB111 or capped with O-Methyl.

In some embodiments, the imaging moiety comprises a CT contrast agent. In some embodiments, the CT contrast agent comprises iodine-based moieties (iodine-substituted aryl moiety (which may comprise one or more aryl groups, e.g., two fused or covalently associated aryl rings, such as ioxehol, tri-iodo phenyl, or N-acetyl iopanoamide) comprising between 1 and 6 iodine atoms, lanthanide-based contrast agents, gold-based moieties and heavy metal-based contrast agents (tantalum and bismuth nanoparticles). In some embodiments, the CT contrast agent comprises a polymer such as a polyamine (e.g. a polymeric nanoparticle, a dendrimer, such as PAMAM dendrimer, etc.) covalently bound to an iodine-based moiety.

In some embodiments, the CT contrast agent comprises a metal nanoparticle; optionally wherein said metal nanoparticle is a gold-nanoparticle (GNP).

some embodiments, the CT contrast agent is a lanthanide-based moiety. Lanthanides with high atomic numbers may be used as CT contrast agents. Among the lanthanides, gadolinium has been most intensively studied for biomedical applications because it is also used as a MRI contrast agent due to its paramagnetic property. Since free lanthanide ions are very toxic, chelating agents such as diethylenetriamine pentaacetic acid (DTPA) and 1,4,7,10-tetraazacyclododecane-1,4,7,10-tetraacetic acid (DOTA) may be employed to reduce the toxicity, and several Gd-chelates are approved by the FDA. In addition, gadolinium may be in the form of gadolinium nanoparticles, optionally coated. In some embodiments, the lanthanide is not gadolinium.

In some embodiments, the CT contrast agent is a bismuth-based moiety. Bismuth-based contrast agents are explored for in vivo use as an alternative to GNPs. Bismuth has a high atomic number (Z=83) and X-ray attenuating properties (absorption edge k=91) and is used as an ingredient in pharmaceuticals and cosmetics. Since metallic bismuth is too reactive to be used in vivo, Bi2S3 nanoparticles coated with polyvinyl pyrrollidone (PVP) may be used as CT contrast agents. Chelating agents such as diethylenetriamine penta-acetic acid (DTPA) and 1,4,7,10-tetraazacyclo dodecane-1,4,7,10-tetraacetic acid (DOTA) may be used with Bi.

In some embodiments, the CT contrast agent is a gold-based moiety or a moiety comprising at least one gold metal atom (non-ionic). The gold-based moiety may be a plurality of gold nanoparticles (GNP), each being a colloidal gold nanoparticle that is non-toxic and non-immunogenic.

In some embodiments, the probe is an in vivo probe. In some embodiments, the probe is an in vivo CT-imaging probe. In some embodiments, the in vivo CT-imaging probe comprises a gold nanoparticle (GNP) as the CT contrast agent.

In some embodiments, the GNP is selected to have an average size (average diameter) of between about 5 nm to about 200 nm. In some embodiments, the GNP is selected to have an average size (average diameter) of between about 10 nm and about 100 nm. In some embodiments, the GNP diameter is on average about 10 nm. In some other embodiments, the GNP diameter is on average about 30 nm. In some further embodiments, the GNP dimeter is on average about 100 nm.

The GNPs may be surface-modified or functionalized by surface ligands, e.g., with multiple tumor markers such as antibodies, peptides or small molecules. In general, the targeting efficacy of the functionalized nanoparticles may depend on the nature of the ligand, the selected coupling reaction (or coupling moiety) and the ligand surface density.

In some embodiments, the in vivo CT-imaging probe comprises a gold nanoparticle (GNP) coated with polyethylene glycol. In some embodiments, the gold nanoparticle is conjugated with polyethylene glycol. In some embodiments, the polyethylene glycol is a plurality of polyethylene glycol molecules. In some embodiments, at least one polyethylene glycol molecule is attached to GB111. In some embodiments, at least one polyethylene glycol molecule attaches GB111 to the gold nanoparticle.

In some embodiments, at least one of polyethylene glycol molecule is capped with O-methyl. In some embodiments, O-methyl is an O-methyl group. In some embodiments, polyethylene molecule not attached to GB111 are capped with O-methyl. In some embodiments, the GB111 and O-methyl are at the end of molecule that is not the end that contacts the nanoparticle. In some embodiments, contacts is adhered to.

In some embodiments, the in vivo CT-imaging probe is represented by:

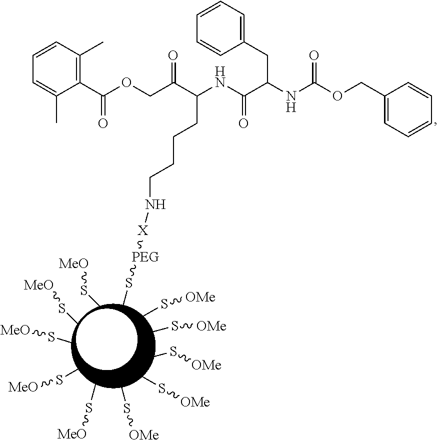

wherein a wavy bond represents PEG, and wherein X is a bond.

In some embodiments, X is

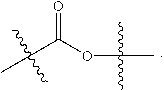 [SMILES: *OC(*)=O]

Once the likelihood of being non-responsive to the immune-checkpoint inhibition therapy is established a new therapeutic regime may be designed.

Thus, the present invention concerns a method for treating a subject being a candidate for anti-cancer immunotherapy the method comprising:
a. Establishing the likelihood of the subject being non-responsive to immunotherapy by the method described above.
b. In a subject found to have a high likelihood of being non-responsive:
   Ceasing or not starting the anti-cancer immunotherapy all together
   Increasing the immune therapy (by dose, frequency, or both)
   Adding to the immune therapy another anti-cancer therapy (chemotherapy, radiation surgery)
   Potentiating the immune check point therapy by administration of a potentiating agent.

In some embodiments, the methos described above is a methos of the invention.

In some embodiments, the method further comprises administering the anticancer immunotherapy treatment to a subject that is likely to respond. In some embodiments, a subject that is likely to respond is not a subject that is unlikely to respond. In some embodiments, the anticancer immunotherapy treatment is administered to a subject with a cathepsin level at or below the predetermined threshold level.

In some embodiments, the method further comprises administering to a subject determined to be unlikely to respond to a higher dose of the treatment. In some embodiments, a higher dose is higher than a standard dose. In some embodiments, a standard dose is the dose that is administered to a responder. In some embodiments, the method further comprises administering to a subject determined to be unlikely to respond a more frequent dose. In some embodiments, a more frequent dose is a more frequent dose schedule. In some embodiments, more frequent is more frequent than a standard frequency. In some embodiments, a standard frequency is the frequency that is administered to a responder. In some embodiments, the method further comprises administering to a subject determined to be unlikely to respond the treatment in combination with a second anticancer agent. In some embodiments, the second anticancer agent is not an immunotherapy. In some embodiments, the second anticancer agent is a second anticancer therapy. In some embodiments, the second anticancer therapy is a standard therapy. In some embodiments, a standard therapy is a first line therapy. In some embodiments, the second anticancer therapy is selected from chemotherapy, radiation therapy and surgery. In some embodiments, the method further comprises administering to a subject determined to be unlikely to respond the treatment in combination with a potentiating agent. In some embodiments, the potentiation agent increases effectiveness of the treatment. In some embodiments, the potentiation agent is at least one cathepsin inhibitor. In some embodiments, the potentiation agent is a molecule of the invention. In some embodiments, the potentiation agent is a conjugate of the invention.

By a preferred option the potentiation of the immune checkpoint therapy is by the administration of at least one cathepsin inhibitor. A list of cathepsin inhibitors can be found in, for example, Siklos et al., “Cysteine proteases as therapeutic targets: does selectivity matter? A systematic review of calpain and cathepsin inhibitors”, Acta Pharmaceutica Sinica B 2015; 5 (6): 506-519; and Pišlar, et al., “The role of cysteine peptidases in coronavirus cell entry and replication: The therapeutic potential of cathepsin inhibitors”, PLOS Pathog. 2020 Nov. 2; 16 (11): e1009013, both of which are incorporated herein by reference in their entirety. In some embodiments, the cathepsin inhibitor is a molecule of the invention. In some embodiments, the cathepsin inhibitor is a cathepsin inhibitor of the invention. A preferred inhibitor is GB111-NH2 of the formula C33H39N3O6. In some embodiments, the at least one cathepsin inhibitor is GB111-NH2. GB111-NH2 is represented in the formula:

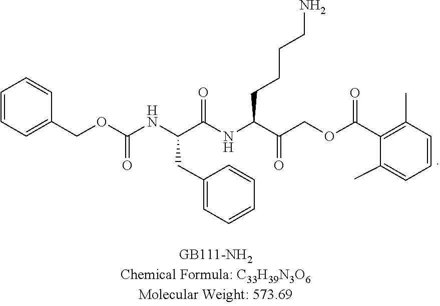 [SMILES: Cc1cccc(C)c1C(=O)OCC(=O)[C@H](CCCCN)NC(=O)[C@H](Cc1ccccc1)NC(=O)OCc1ccccc1]

By another option the present invention concerns a method for treating a subject eligible for anticancer immunotherapy, the method comprising:
a. determining if the subject is non-responsive to treatment with the anticancer immunotherapy; and
b. if (a) is affirmative, adding to the anti-cancer immunotherapy at least one cathepsin inhibitor agent.

Typically, the determination of (a) is by the method of the invention that measures cathepsin level or activity. The method may also measure other parameters relating to responsiveness of the immune system. If the subject is found non-responsive to the immunotherapy treatment, he should be administered also with at least one cathepsin inhibition agent as described above preferably an inhibitor of B, L and S cathepsins). The administration of the agents may be together with administration of the anticancer immunotherapy drug, may be before beginning of therapy, after beginning t of therapy or in between administration cycles of the anticancer immunotherapy.

By another aspect, there is provided a method of treating a subject non-responsive to immunotherapy, the method comprising administering to the subject the immunotherapy and a cathepsin inhibitor, thereby treating a subject non-responsive to immunotherapy.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R—) or (S—) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded, and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol. Additional substituents are disclosed herein. Further, the term “substituted” encompasses or more (e.g., 2, 3, 4, 5, 6, or more) substituents, wherein the substituent(s) may be same or different, and wherein each of the substituents is as described herein.

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

In some embodiments, the term “carbocyclyl” comprises an aryl, a polycyclyl, a heteroaryl, a cycloalkyl, or heterocyclyl or any combinations thereof.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein. or “carboxylate”

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)₂R′ group, where R′ is as defined herein.

A “carbamyl” or “carbamate” group describes an —OC(O)NR′R″ group, where R′ is as defined herein and R″ is as defined for R′. A “nitro” group refers to a —NO₂ group. The term “amide” as used herein encompasses C-amide and N-amide. The term “C-amide” describes a —C(O)NR′R″ end group or a —C(O)NR′— linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′as defined hereinabove. The term “guanidine” describes a —R′NC(N)NR″R′″ end group or a —R′NC(N)NR″— linking group, as these phrases are defined hereinabove, where R′, R″ and R″ are as defined herein. As used herein, the term “azide” refers to a —N₃ group. The term “sulfonamide” refers to a —S(O)2NR′R″ group, with R′ and R″ as defined herein.

The term “phosphonyl” or “phosphonate” describes an —OP(O)—(OR′)₂ group, with R′as defined hereinabove. The term “phosphinyl” describes a —PR′R″ group, with R′ and R″ as defined hereinabove. The term “alkylaryl” describes an alkyl, as defined herein, which is substituted by an aryl, as described herein. An exemplary alkylaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2 (1H)-quinolinonyl, 4-oxo-1,4-dihydroquinolinyl, 2H-1-oxoisoquinolyl, 1,2-dihydroquinolinyl, (2H) quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H) quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]pyridyl, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, O—C₁₋₆-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine or iodine, also referred to herein as fluoride, chloride, bromide and iodide.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption. Lists of pharmaceutically acceptable salts may be found, e.g., in Remington's Pharmaceutical Sciences, 17th ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### General

As used herein the term “about” refers to +10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

Determining the TAM content and cathepsin activity in human melanoma tissue from naïve, and resistant IT treated patients

The change in M2 macrophages (MP) and cathepsin activity during immunotherapy including checkpoint inhibitors in human samples was evaluated using a fluorescent cathepsin ABPs that reports on the activity of cathepsin B, L and S. The Cathepsin activity was evaluated in human tumor lysates samples from naïve metastatic melanoma patients and of those that were resistant to IT, by SDS PAGE using the GB123 a cathepsin activity-based probe.

Freshly excised metastatic melanoma human samples were collected under Cleveland Clinic IRB (IRB #3164, PI Brian Gastman), and tissues were processed and analyzed after applying GB123 labelling ex vivo. For FACS, single cell suspension from tumors treated with GB123 washed and analyzed. A combination of antibodies such as (CD45+, CD3- and CD68+) with CD80+ and CD163+ for M1 and M2 macrophages respectively were used and monitored count and cathepsin activity in these cells.

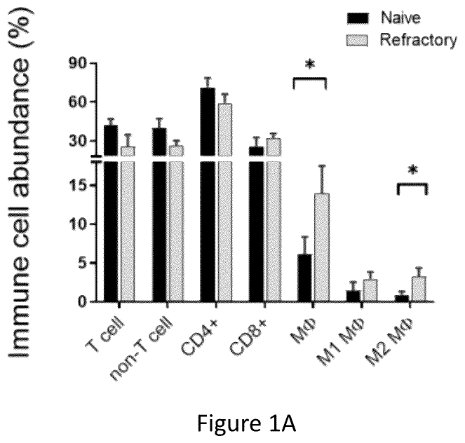

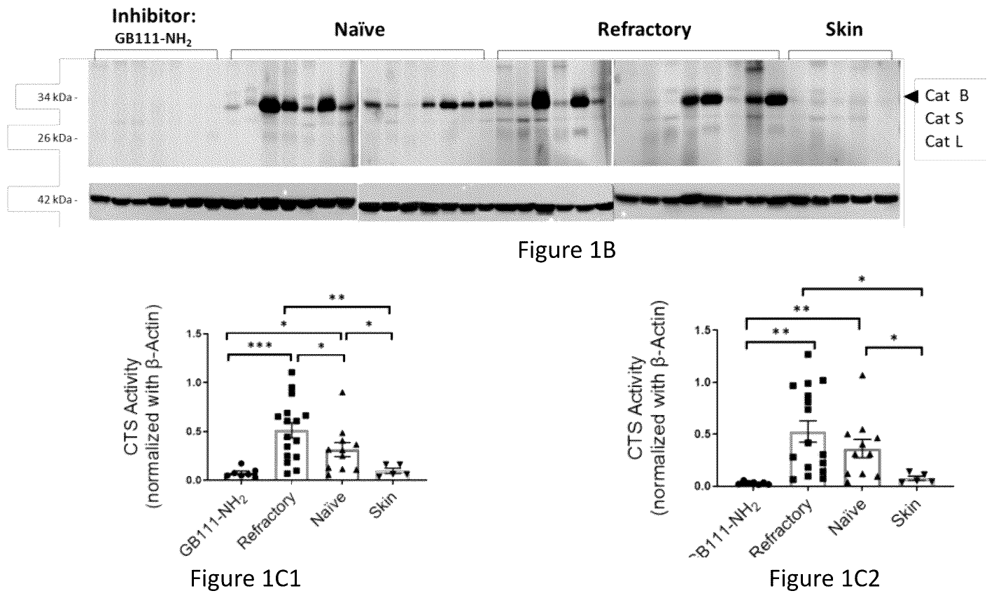

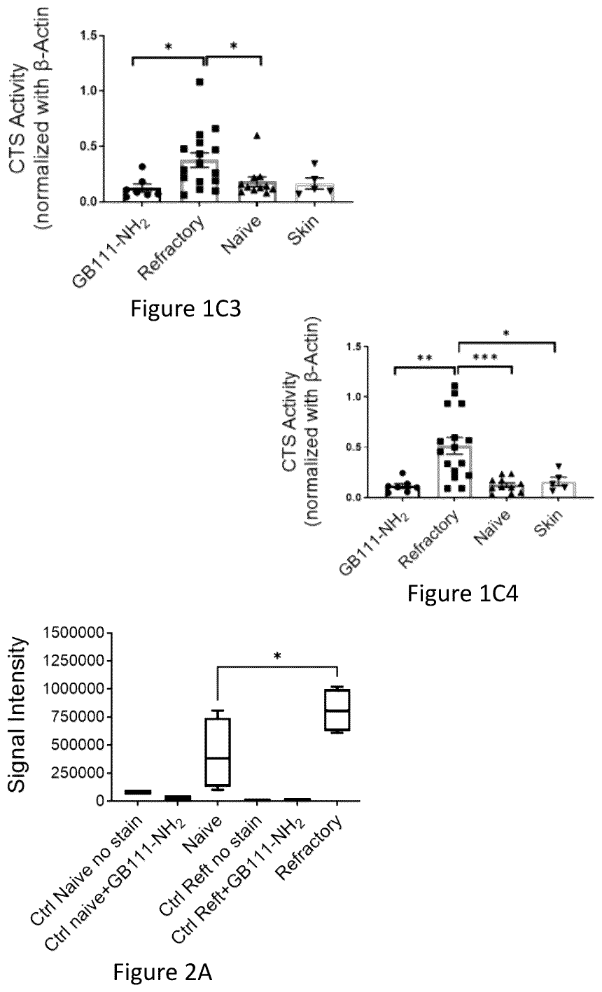

FACS measurements showed that isolated TIL components contain several types of immune cells, but interestingly a significant difference between the naïve and refractory tumors was only seen in the macrophage (MΦ) number population (MΦ and M2 MΦ), which was higher in refractory tumors (FIG. 1A).

A fresh tissue of the samples was collected and flashed frozen in liquid N2, and the tissue lysates were subjected to GB123 labeling. The cathepsin-probe adducts were resolved by gel, enabling the detection of cathepsin B, S, and L activity in the tissue in comparison to adjacent skin's and a non-fluorescent cathepsin inhibitor (GB111-NH2) treated controls (FIG. 1B). Quantification of cathepsin activity in the samples exhibit a significant difference in cathepsin B activity in normal adjacent skin samples and tumor samples. Furthermore, cathepsin S and L isoforms showed a significant difference in the activity between naïve and refractory tumor samples (FIG. 1C1-1C4). Thus, proving that the activity of cathepsin enzymes could differentiate between normal skin, naïve and refractory tissues, as well as the potential of the GB123 probe as a marker for IT resistance.

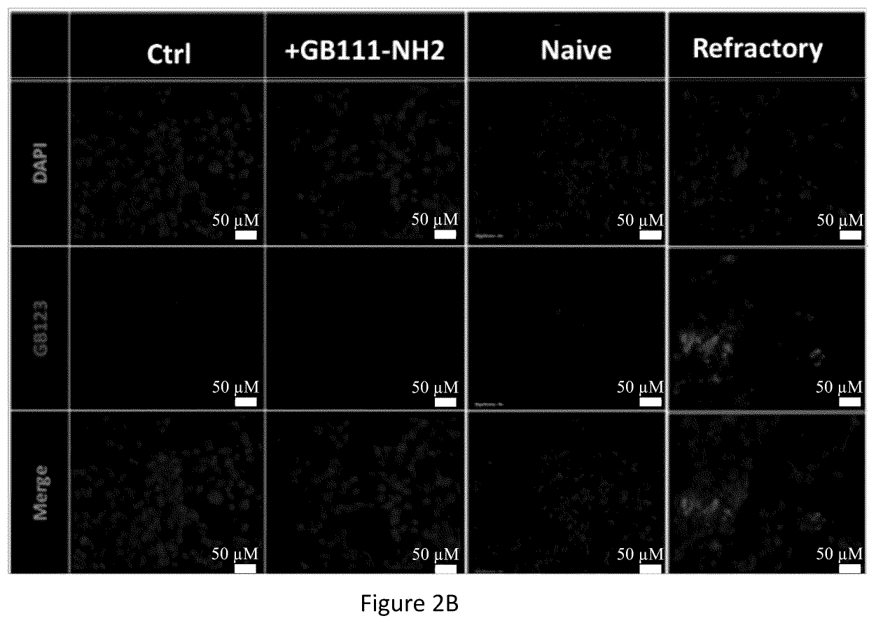

Further corroboration was obtained using fluorescent microscopy analysis. A fresh tissue section was embedded in OCT, cryo-sectioned, and treated with GB123 probe and specific antibodies for cell markers (M1/M2, and T cells). This analysis not only provides information of the GB 123 probe binding to the enzyme activity, but also exhibits the number and location of the cathepsin enzyme producing by MP. The fluorescent quantification of GB123 (Cy5) showed a significantly higher signal intensity in the refractory tissue sample compared to the naïve sample (FIG. 2), confirming that the cathepsin enzyme activity is more pronounced in refractory sample as well as GB123 is applicable as a detection marker for the cathepsin enzyme.

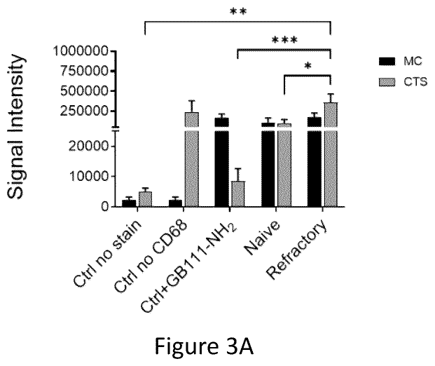

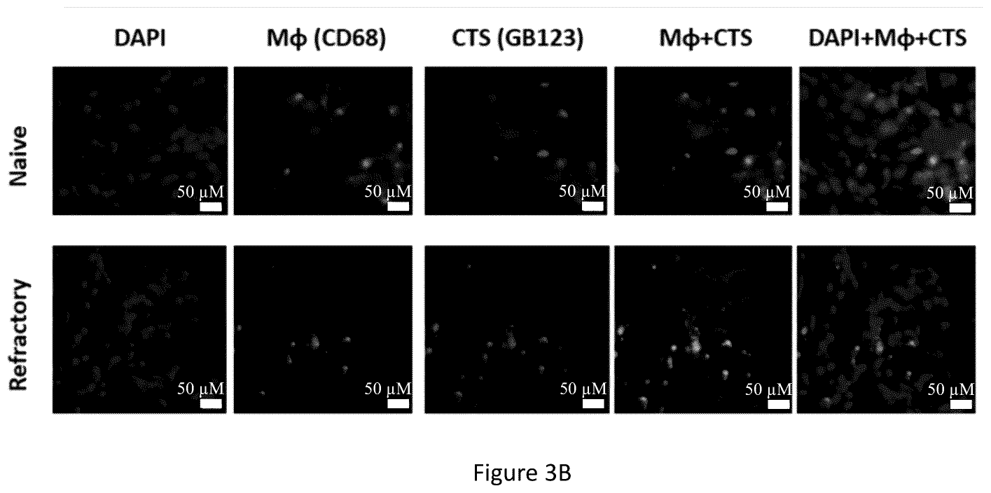

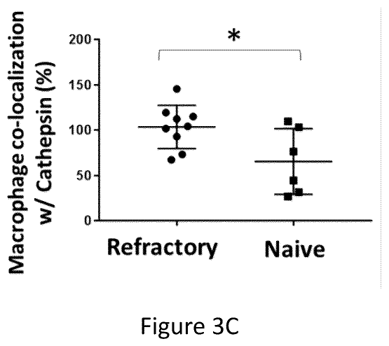

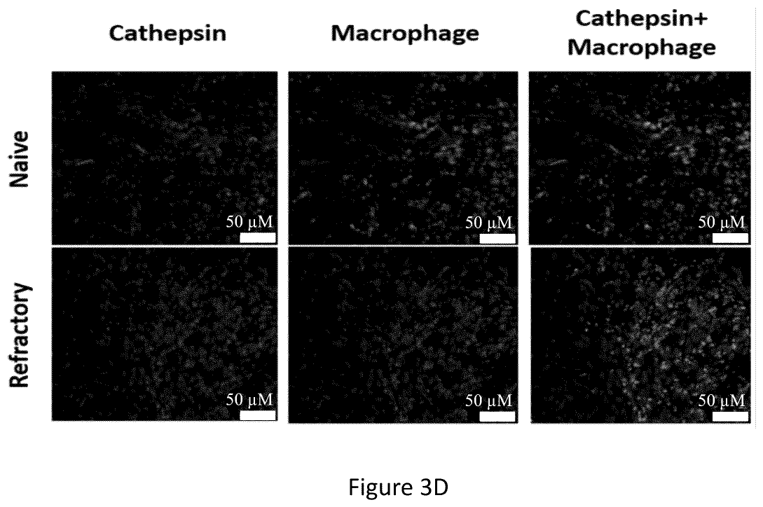

The inventors further examined if the elevated activity detected in human melanoma tissues are mainly produced by macrophages. Therefore, tissue sections were co-stained with GB123 and with a macrophage marker, CD68 that binds to both (M1 and M2 macrophages). The activity of cathepsins was significantly higher in refractory tumors tissue compared to the naïve, and the sample treated with cathepsin inhibitor, GB111-NH2 (FIGS. 3A-3B). Furthermore, the co-localization of the MP and cathepsin activity, changes according to the patient's response to therapy. These results reflect that a higher amount of cathepsin enzyme are produced from MP (probably produced from MΦ M2) in the refractory tumor compared to the naïve tumor (FIGS. 3C-3D).

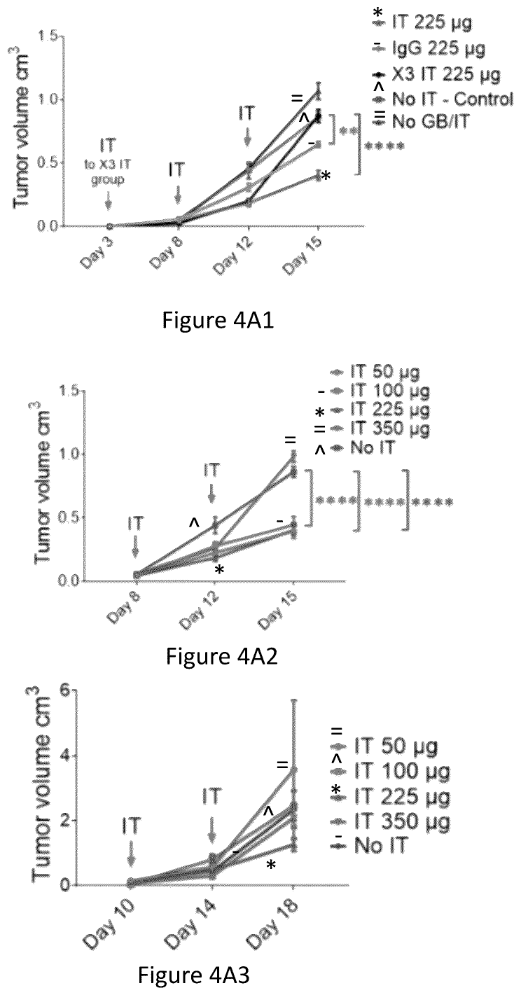

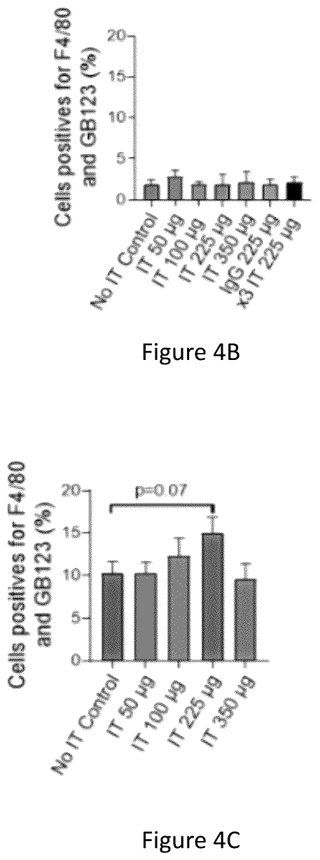

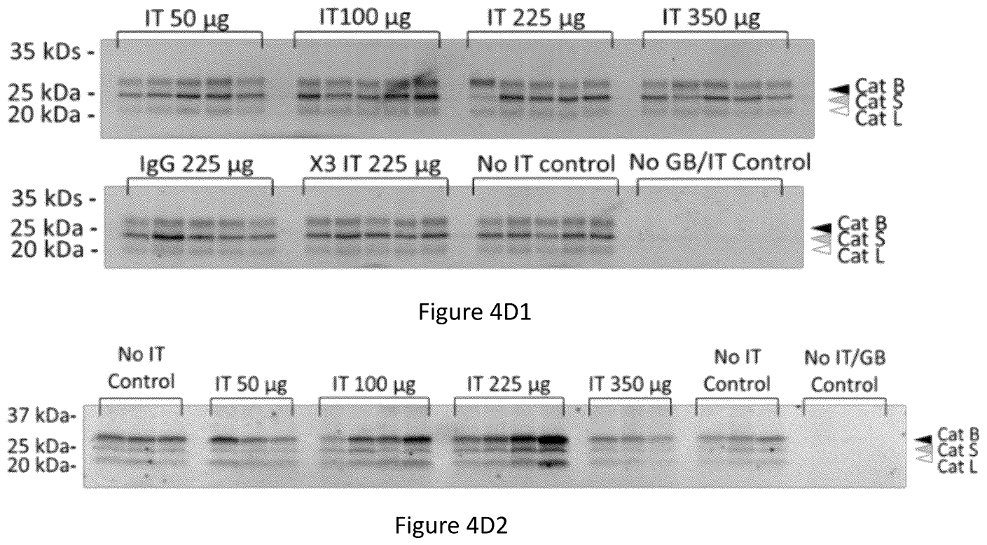

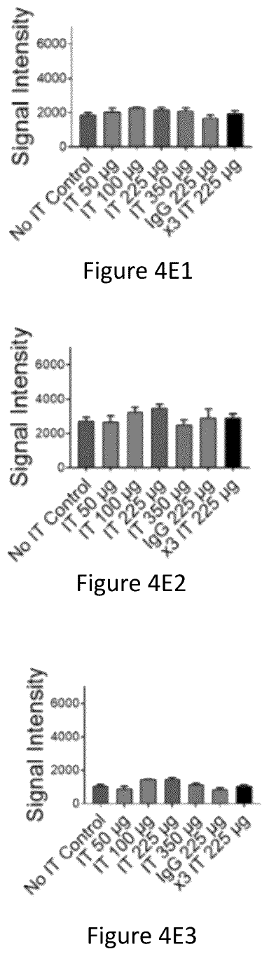

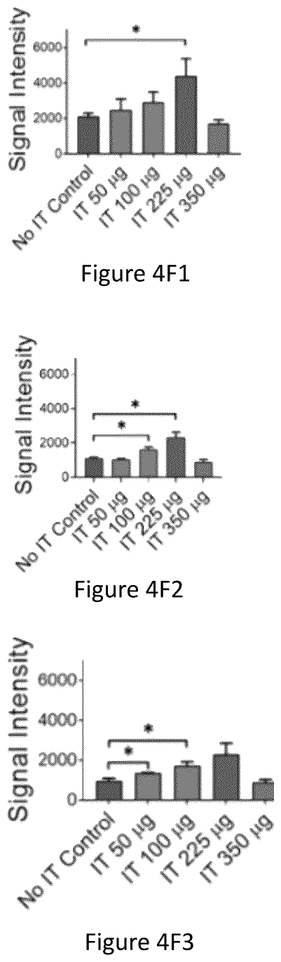

To further show that changes in M2 macrophages and cathepsin activity during immunotherapy (IT) treatment are involved in promoting resistance, two melanoma in vivo models were established. A sensitive IT treatment model, D4M tumors and, a more resistant model, IT B16-F10 tumor Cells were injected subcutaneously (SC) to the back of the mice and their growth was measured continuously by caliper. Anti PD-1 was administered intra peritoneally (IP) twice, a few days after the tumor could be palpated, and on indicated days and doses as described in the methods, changes in tumor volume were measured during the experiment (FIG. 4A).

As expected, the sensitive D4M tumors responded to treatment, their tumor volume was significantly reduced with IT in comparison to the control group, they showed no visible necrotic tissue and grow slower than the B16-F10 tumors. Whereas, the B16-F10 tumors were much more aggressive and there was a high variance between the tumor size, in addition, the tumors ulcers and there was necrotic tissue in the tumor core.

The IT treatment doses in both resistant melanoma model and sensitive model was examined, as anticipated the B16-F10 tumors were less responsive to the IT treatment. In addition, surprisingly, a slight reduction in tumor volume was detected with IgG treatment, though it was less than the reduction produced by IT treatment (FIG. 4A).

To determine the cathepsin activity in both models, one day prior to sacrifice, GB123 (a fluorescent cathepsin activity-based probe) was administered IV, and tumors were harvested for further biochemical examination. Tumor tissue was subjected to fluorescence-activated cell sorting (FACS) analysis to determine the percentage of tumor associated macrophages (TAMs) that express high cathepsin activity. The responding tumors (D4M) had a low percent (2-4%) of macrophages expressing high cathepsin activity (FIG. 4B), while the resistant B16 tumors had a higher percent of cathepsin positive TAMs (9-15%). Furthermore, an increase in the percentage of cathepsin positive TAMs with IT dose was detected in the B16 tumors while the D4M tumors, had similar percentages throughout all treatments (FIGS. 4B-4C).

These results were further confirmed by SDS PAGE as reflected by the intensity of the cathepsin-GB123 fluorescently labeled band of the cathepsin B, L and S activities. The D4M tumors present constant cathepsin activity in all IT concentrations, similar to the control group (FIG. 4D). In the quantification of individual cathepsin activity in D4M tumors, no significant differences in cathepsin activity were observed (FIG. 4E).

In B16-F10, the cathepsin activity rose as the IT concentration increased (up to 225 μg/dose), surprisingly, the response to IT 350 μg was different, while the cathepsin activity was significantly lower than 100 μg IT group the macrophage count was higher, this phenomenon emphasized the selection of accurate dose for therapy (FIG. 4F). All three cathepsins, B, L and S activity in B16-F10 tumors showed significant increase in activity with treatment. It is postulated that the increase in cathepsin activity originates from the macrophages in the tumor microenvironment (TME) that are involved in resistance mechanisms.

In addition, the microscopy scans show that not only the cathepsin activity is elevated in a dose-response manner but also the macrophage content is increased in the B16-F10 resistant tumors and in a dose-response manner up to 225 μg while there is no significant difference in tumor size. The D4M samples showed constant cathepsin activity with dramatically lower macrophage count relative to the B16-F10 tumors, indicating that macrophages and in particular their cathepsin activity my drive resistance to IT.

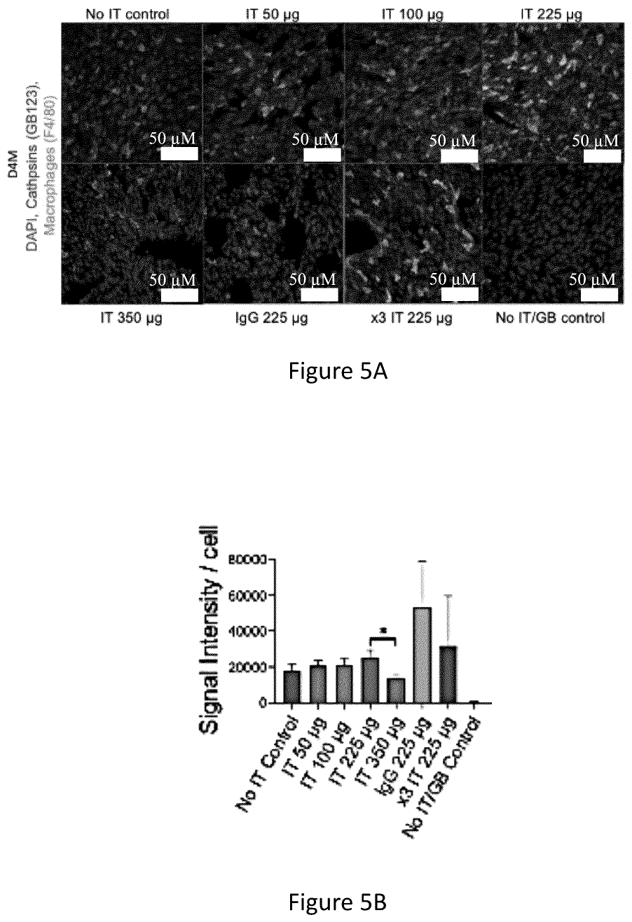

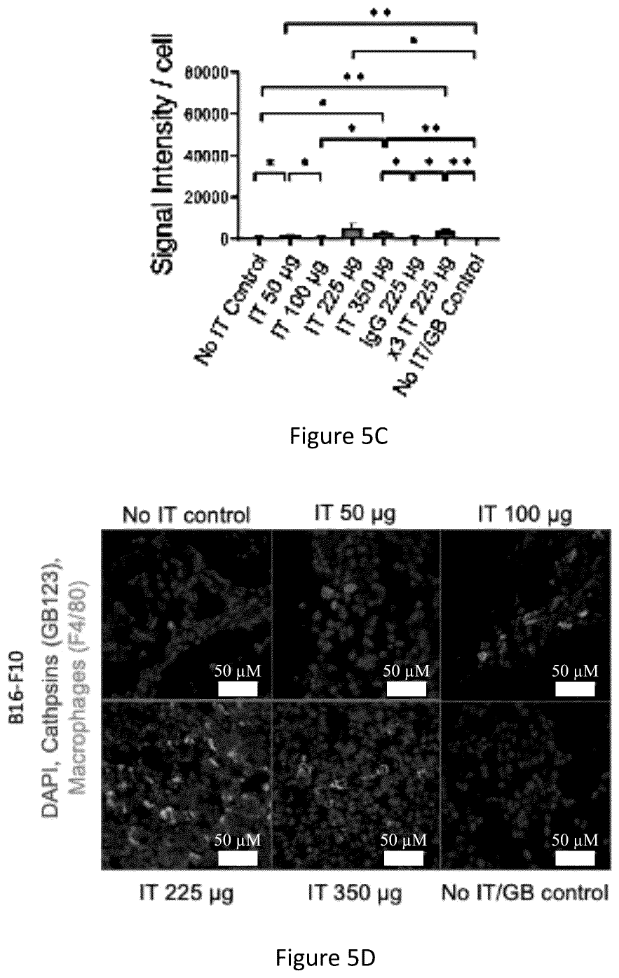

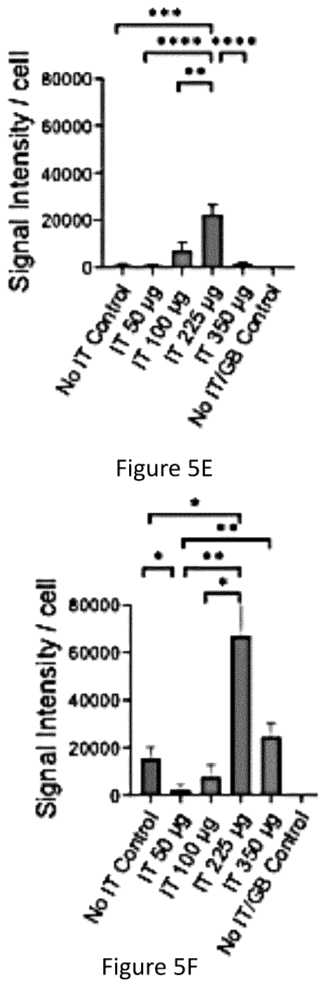

Furthermore, the cathepsin activity was monitored in TAMs by placing fresh resected tumor tissues in OCT block and subjecting it to cryosectioning, slides were then they were fixed and stained with a Cy3-F4/80 macrophage antibody, and the cathepsin activity was fluorescently labeled in vivo by the Cy5-GB123 cathepsin probe (FIG. 5). Microscopy images of D4M tumors show that the cathepsin activity is easily detected and is constant between treated mice and the control without IT treatment (with the exception of 350 μg/dose) (FIG. 5A). Quantification of fluorescent signals of Cy5, GB123 signal, and Cy3, F4/80 signals correspond well with the images (FIGS. 5B-5C). While the cathepsin signal is steady between the groups the macrophage content varied significantly between treatment groups (FIG. 5C). In general, the B16-F10 showed lower fluorescence as these are black tissues that absorb part of the fluorescence signal. Microscopy images of B16-F10 tumors show an increase in cathepsin activity with the IT dose (FIG. 5D). Quantification of fluorescent signals show a significant gradual increase in cathepsin activity in a dose-response manner relative to the non-treated controls.

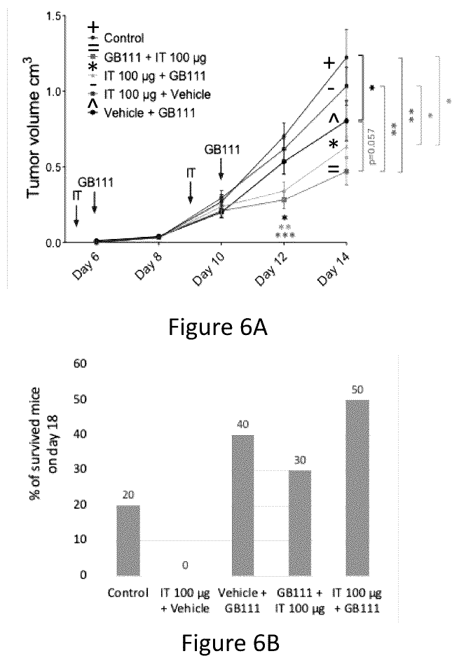

The effect of the inhibition of cathepsin activity on the resistance to IT was examined using the resistant B16-F10 tumor-bearing mice with IT (anti-PD-1), GB111-NH₂ (cathepsin inhibitor), or their combinations. The B16-F10 tumor-bearing mice were treated every 4 days, starting on day 5 after cell inoculation with anti PD-1 (100 μg/dose), GB111-NH2 (30 mg/kg) or their combinations; IT was given a day before GB111-NH2 (a group of GB111-NH2 first and IT a day later was included). Tumor size was measured over the course of the experiment (FIG. 6A), Tumor volume (cm³) show significantly smaller tumors in mice treated with the combination of a cathepsin inhibitor and the IT (anti-PD-1) treatment. In addition, survival plots were generated (FIG. 6B).

### Example 2—Antibody-Drug Conjugate

### Materials and Methods

### Competitive Inhibition

Recombinant cathepsin B and L (rCatB and rCatL, respectively) were incubated for 45 minutes at 37° C. with 1 μl of increasing concentrations of cathepsin inhibitors (GB111-NH2 or MGB, respectively), residual cathepsin activity was labeled by addition of 1 μM of GB123 fluorescent activity-based probe for 45 minutes at 37° C., sample buffer was added for 2 min and the sample was boiled at 100° C. for 2 minutes. Equal amount of each sample was separated by a 12.5% SDS PAGE, this experiment was repeated 3 times. The gel was scanned for fluorescence by a Typhoon scanner. The bands were quantified by ImageJ software and then IC50 curves were generated from triplicate experiment by Prism-GraphPad software.

### Cathepsin Inhibition Cells

2.5*105 cells per well (A549, U87 or HepG2), were seeded in 6 well plates one day before incubation with indicated concentrations of inhibitors (GB111-NH2 or MGB, respectively) for 1 hour at 37° C. Residual cathepsin activity was labeled by the addition of 2 μM of GB123 for 2 hours at 37° C. Sample buffer was added and samples were boiled at 100° C. for 10 min. Equal protein amounts of each sample were resolved by a 12.5% SDS PAGE. a) Gels were scanned for fluorescence by a Typhoon scanner. b) The gel bands were quantified using ImageJ software and IC50 curves were generated from triplicate experiments using Prism-GraphPad software.

### Immunoprecipitation

A total of 100 μg of cell lysate were treated with an anti-cathepsin L antibody (Cathepsin L RD-06-0007). Fluorescently labeled cathepsin L was immune precipitated using protein A/G beads, beads were washed and cathepsin L was eluted by boiling with sample buffer. Samples were separated by SDS PAGE and scanned for fluorescence.

### Cell Viability Assay

Indicated cells (Hacat, U87, A549 or HepG2) were seeded in 96 well plates, 2500 cells per well a day prior to the addition of the cathepsin inhibitors: ACE2-ab-MGB (an exemplary conjugate of the invention), MGB or GB111-NH2. The cells were incubated with compounds at indicated concentrations for 72 hours at 37° C. and then fixed by 2.5% glutaraldehyde for 1 hour at room temperature and stained with methylene blue. Growth was compared to vehicle-treated cells designated as 100%.

### Optimization of Antibody Drug Ratio

Intact A549 cells, 2.5*10\hat{ }5 per well, were seeded in 6 well plates one day pretreatment. Cells were incubated for 1 hour at 37° C. with different ADCs each with a different number of MGB molecules per each antibody, (the MGB was kept at 2 μM while the antibody concentration increased). Residual cathepsin activity was labeled by the addition of 2 μM of GB123 for 2 hours at 37° C. Sample buffer was then added, and samples were boiled for 10 min at 100° C. Equal protein amounts of each sample were resolved by a 12.5% SDS PAGE.

### The Synthesis Process of MGB

The initial cathepsin inhibitor GB111-NH2 was modified to form MGB by solid-phase peptide synthesis where the carbobenzoxy group of GB111-NH2 was replaced with maleimide moiety which acts as the attachment point to a thiol-containing substance such as an antibody. Furthermore, MGB has an additional phenylalanine amino acid residue, which renders less cell permeability as well as increases binding interactions, while maintaining its potency.

The synthetic pathway is represented in scheme 1. In general, the commercial Fmoc and Boc protected lysine bromomethyl ketone was conjugated to dimethyl benzoic acid using KF as a mild base. The Boc was removed from the newly synthesized acyloxymethyl ketone (warhead) and the compound was linked to chlorotrityl resin.

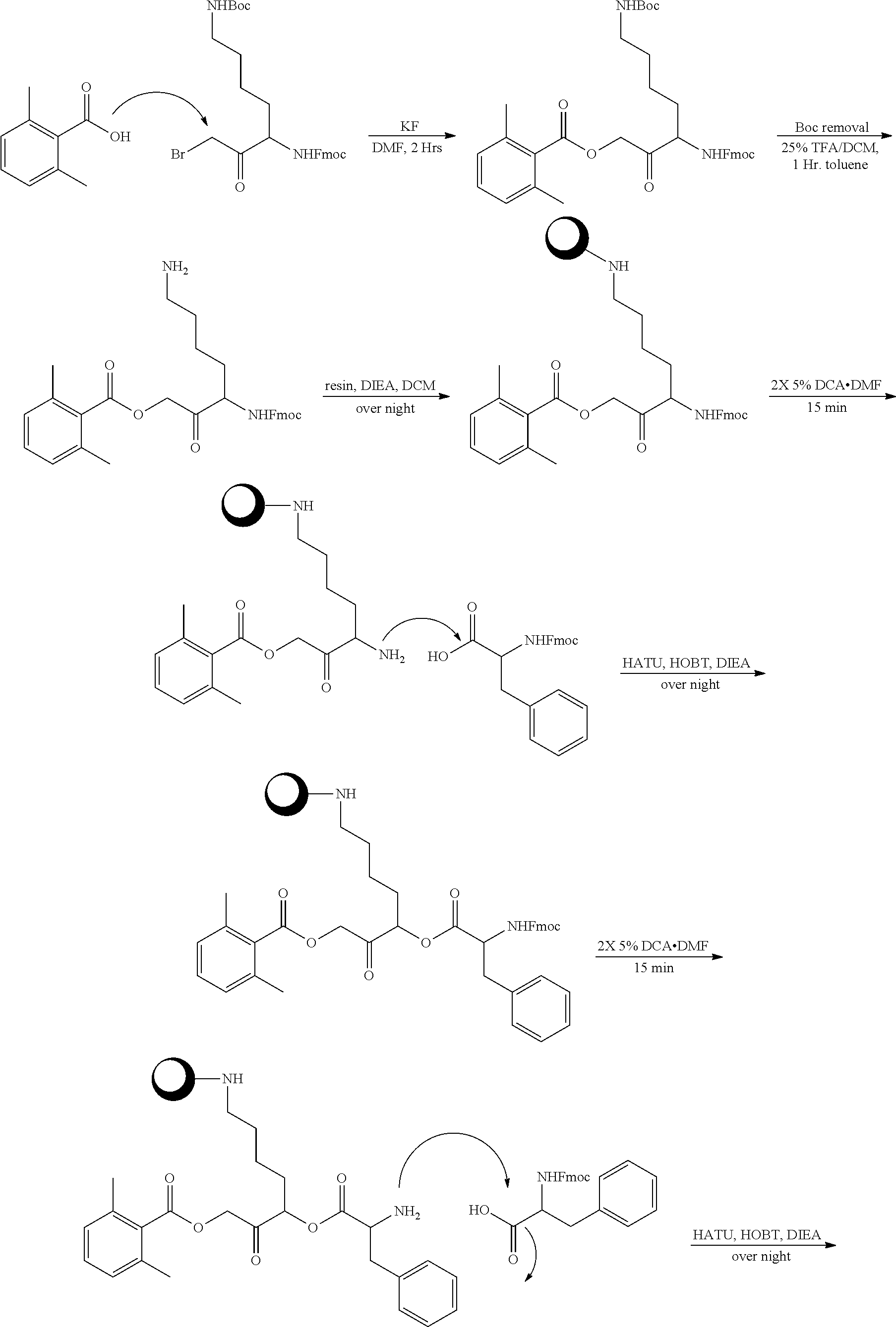

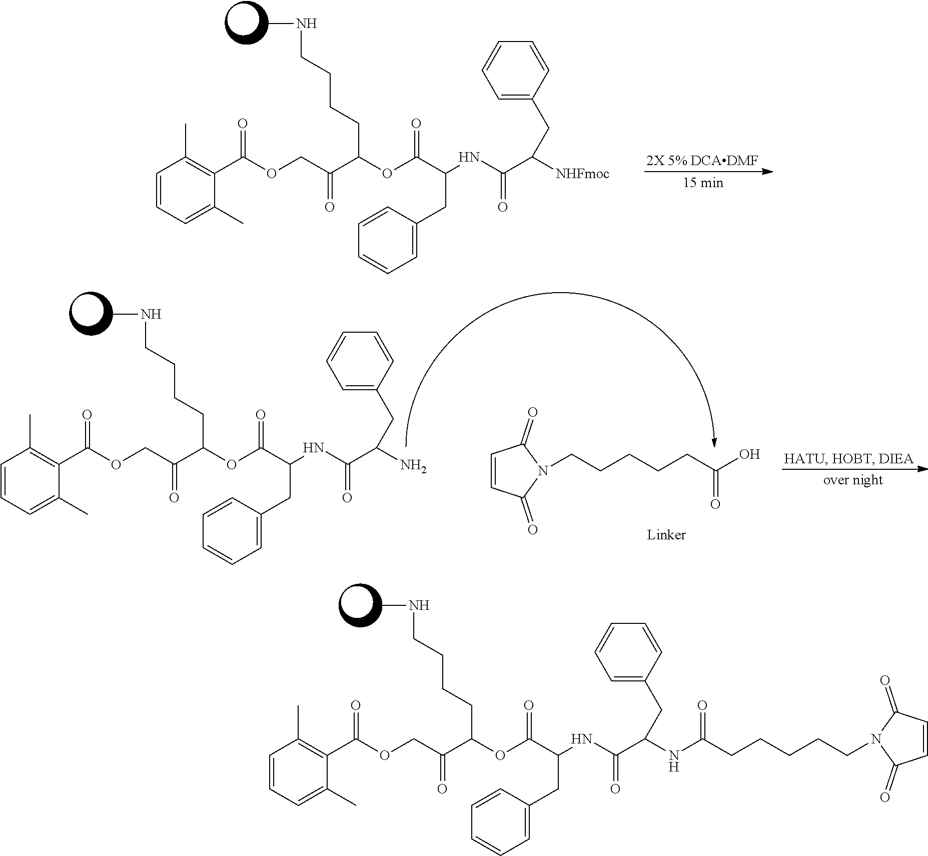

Scheme 1. The synthetic pathway for the synthesis of MGB on the solid support.

Subsequently the MGB on the solid support was cleaved by subjecting thereof to a standard TFA-based cleavage cocktail followed by purification (e.g. HPLC-based purification), to obtain:

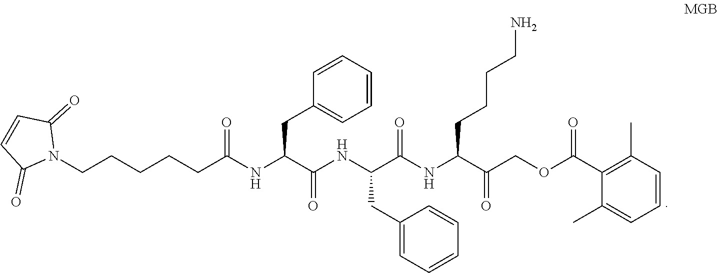 [SMILES: Cc1cccc(C)c1C(=O)OCC(=O)[C@H](CCCCN)NC(=O)[C@H](Cc1ccccc1)NC(=O)[C@H](Cc1ccccc1)NC(=O)CCCCCN1C(=O)C=CC1=O]

The MGB was conjugated to ACE-2 antibody (abbreviated by “ACE2-ab”) by agitating MGB and the ACE2-ab at 37C° for 30 minutes in PBS using a molar excess of MGB, resulting in the formation of ACE2-ab-MGB (example conjugate of the invention):

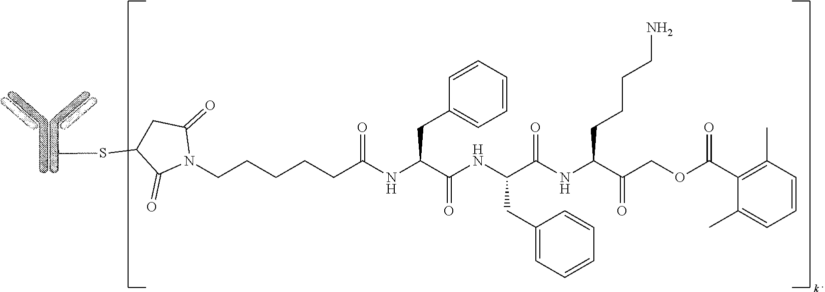

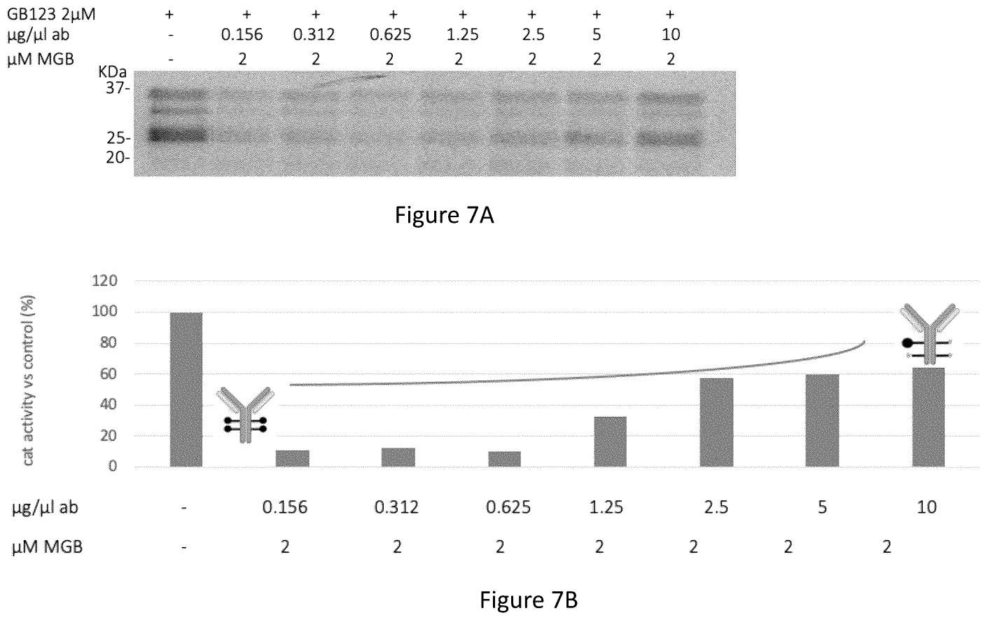

To improve the potency of the ADC in cells the optimal ratio between MGB and the ACE2 antibody was examined. Several conjugated were generated keeping a constant concentration of the drug, MGB, with increasing antibody concentrations. Ratios between 1:3 and 1:200, molar antibody to molar MGB were examined (i.e. various molar excess ratios of MGB). The optimal ratios were found to be between 1:50 and 1:200 molar antibody to molar MGB (1:10-2:5 w/w of antibody to MG) (FIGS. 7A-B).

Despite numerous attempts the inventors couldn't determine the exact MGB: ACE2-ab ratio in the final conjugate. However, it is presumed that the preferable ratio (in terms of inhibition of cathepsin activity) is in the range between 2-20, or between 5-20.

### MGB as an Inhibitor of Cathepsins

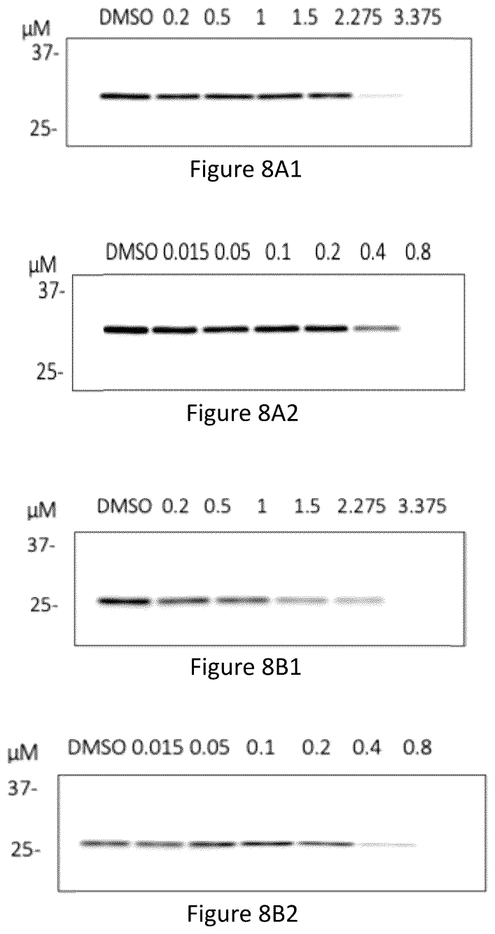

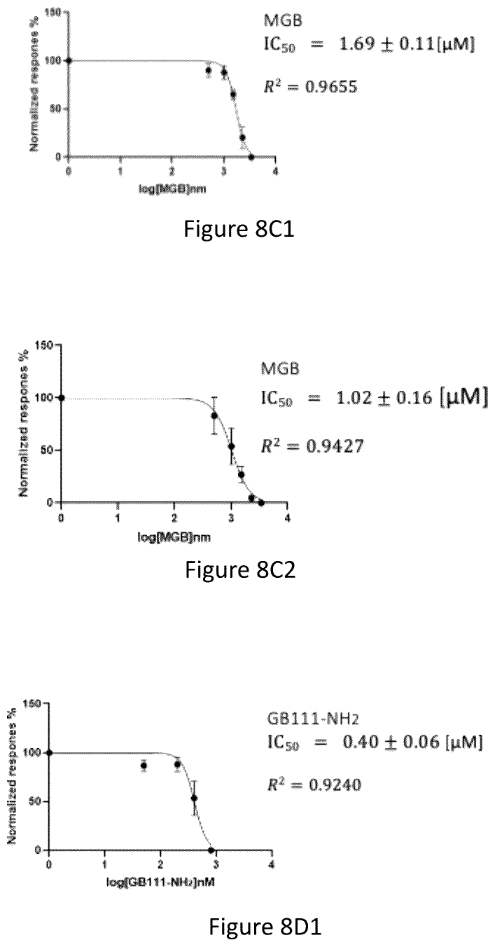

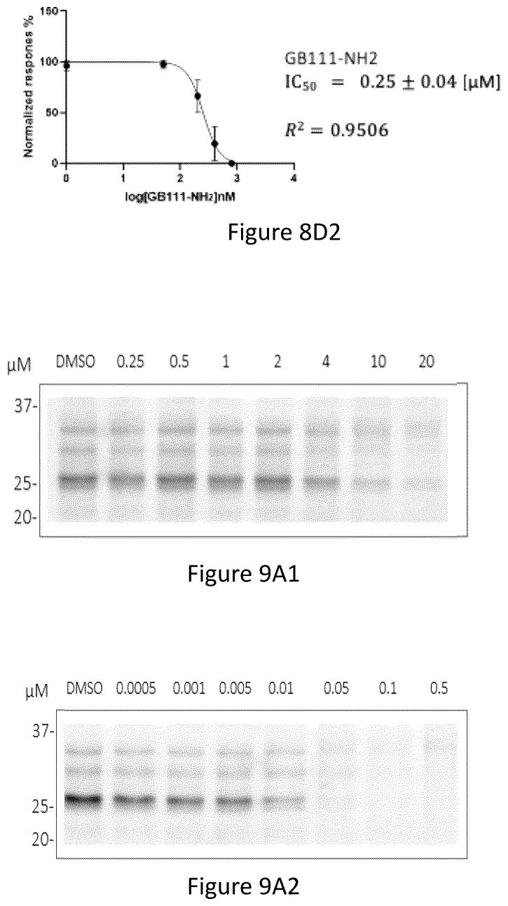

The ability of MGB to act as a cathepsin inhibitor was examined and compared to GB111-NH2 (FIG. 8). Inhibition was detected on SDS-PAGE Potent inhibitors reduce the residual activity and as a result, a reduction in the fluorescently labeled cathepsins is seen (FIG. 8A-8B). MGB inhibits rCatB and rCatL (FIG. 8C). IC50 values show that GB111-NH2 is 4 times more potent than MGB (FIG. 8C-8D).

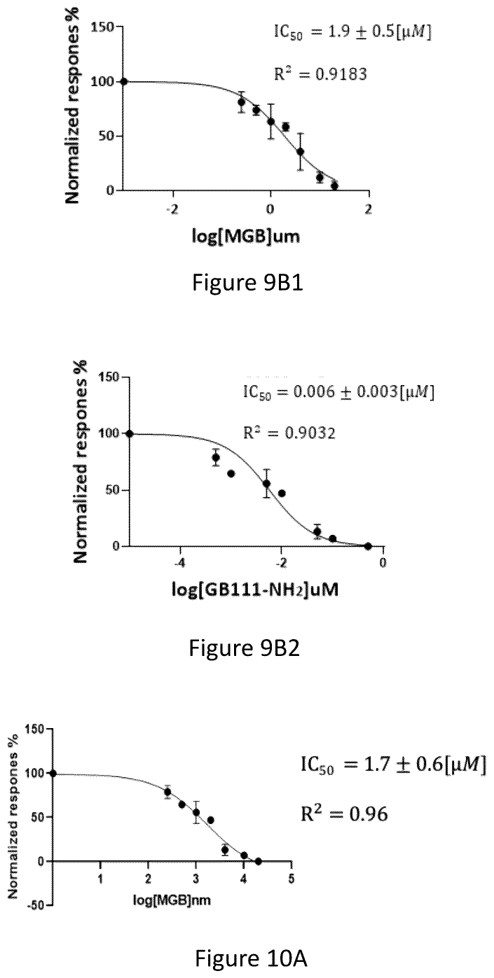

The ability of MGB to penetrate cells and inhibit endogenous cathepsin activity was tested in lung cancer A549 cells. This model (lung cells) was chosen since corona virus is known to infect the respiratory system. Intact cells were treated with inhibitors and the residual activity was determined by GB123. Inhibition was detected on SDS-PAGE Potent inhibitors reduce the residual activity and as a result, a reduction in fluorescently labeled cathepsins is seen (FIG. 9A). The cathepsin activity was quantification and IC50 curves were plotted. A significant difference was observed in the cell permeability of MGB and GB111-NH2. While GB111-NH2 accumulated in lysosomal compartments and inhibited intact cathepsin activity very potently with IC50 of 6 nM, MGB had IC50 of 1.9 μM (FIG. 9B), meaning GB111-NH2 was over 200-fold more potent than MGB.

### The Antibody-Drug Conjugate (ACE2-Ab-MGB) as an Inhibitor of Cathepsins

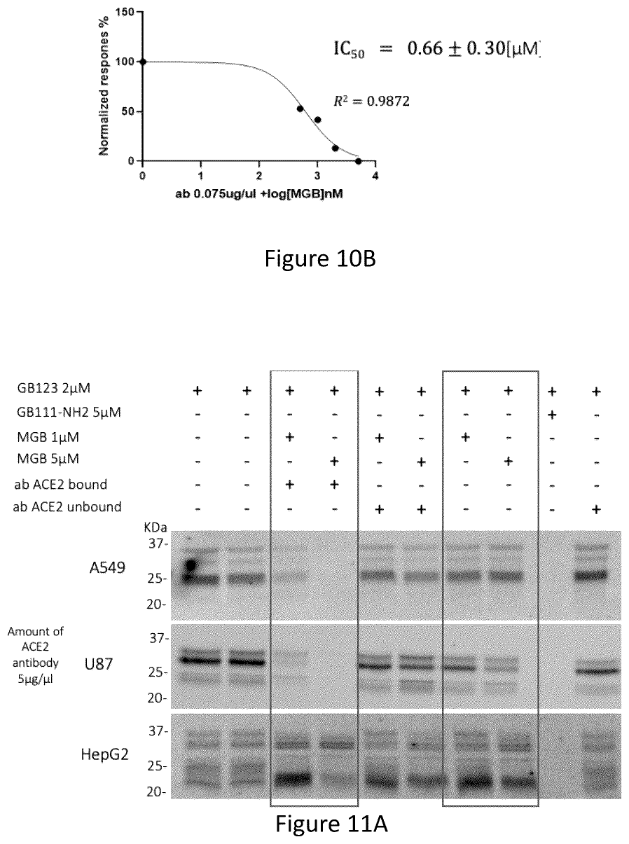

The endogenous inhibition of cathepsin activity of the antibody-drug conjugate ACE2-ab-MGB, was examined and compared to the MGB inhibitor. MGB had an IC50 of 1.7±0.6 μM, the ACE2 antibody conjugated to MGB was more potent with an IC50 of 0.62±0.3 μM (FIG. 10A-10B). The ACE2-ab-MGB is three times more potent than the MGB inhibitor alone.

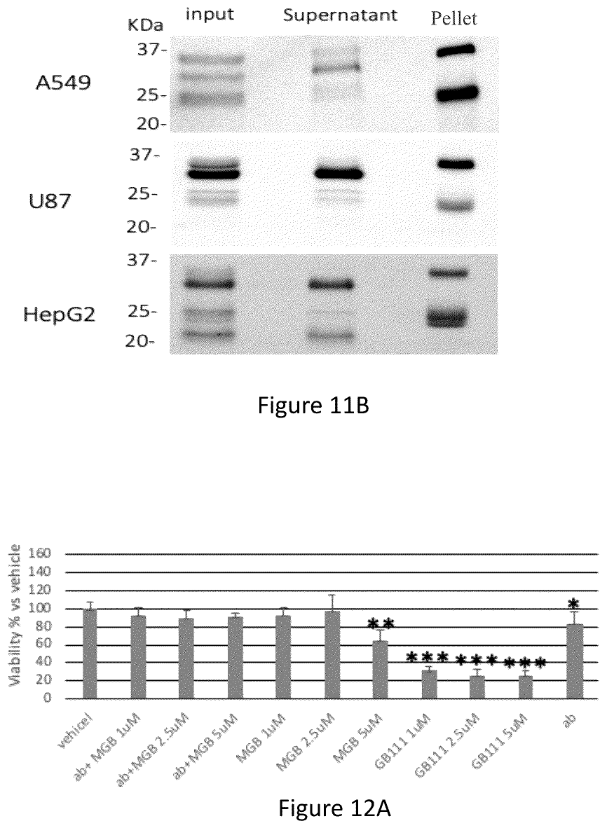

To examine the permeability and selectivity of ACE2-ab-MGB, different cell lines (U87, and HepG2) that express ACE2 were tested. Intact cells were treated with the ACE2-ab-MGB or MGB. In addition, to examine if the linkage between the inhibitor and the antibody is important for the ADC (ACE2-ab-MGB) potency, the inventors treated cells with un-bound ACE2 antibody and MGB in the same concentration. In all three cell lines the ADC (ACE2-ab-MGB) improved cell permeability and potency of the inhibitor relative to the MGB inhibitor alone as well as compared to the unbound inhibitor and antibody (FIG. 11A). In addition, immunoprecipitation was performed in order to identify which of the bands is cathepsin L-all of cell lines express active cathepsin L (FIG. 11B).

### Cell Viability/Inhibitor Toxicity

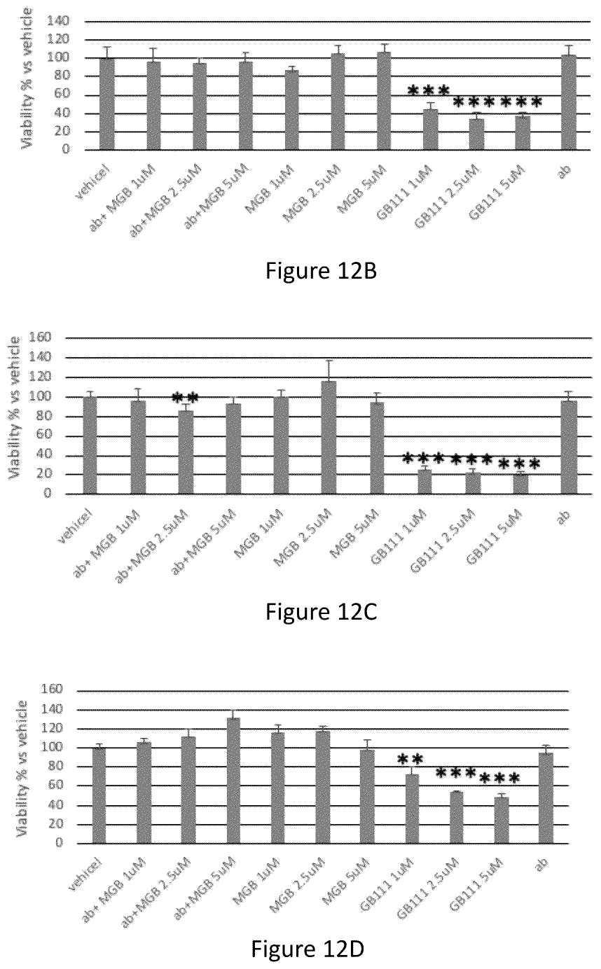

The cell viability after treatment with the inhibitors: ACE2-ab-MGB, GB111-NH2, and MGB was examined and compared in the following cell lines A549, HepG2, U87, and Hacat. As expected GB111-NH2 reduced cell viability significantly even at low concentrations as 1 μM, while the ACE2-ab-MGB and the MGB were safe to cells even at a concentration of 5 μM (FIG. 12).

### Example 3

### Method and Materials

### Cathepsins Activity in Hematological Malignancies Patients

Cell lysates from hematological malignancies patients were pretreated with vehicle, or a cathepsin inhibitor GB111-NH2 for 0.5 hour, then labeled by addition of 1 μmol/L GB123 probe for 1 hour. Equal amounts of protein were separated and visualized by scanning of the gel with a Typhoon flatbed laser scanner. Each lane was taken from a different patient. MZL-Marginal zone lymphoma, CLL-chronic lymphocytic leukemia, DLBCL-Diffuse large B-cell lymphoma.

Crude detergent lysates from cells that were pretreated with either cathepsin inhibitor GB111-NH2 or with control DMSO; vehicle for 20 minutes and then labeled by addition of 1 μmol/L GB123 probe for 1 hour. Equal amounts of protein (see Coomassie) were separated and visualized by scanning of the gel with a Typhoon flatbed laser scanner.

### Immunoprecipitation (IP)

Cells were pretreated with 5 μM GB123, and then lysed in RIPA buffer. Following immunoprecipitation (IP), samples were separated on a 12% SDS PAGE gel and scanned for fluorescence using a Typhoon flatbed scanner.

### Lymphoma Cell Death Assay

OCI-Ly19 DLBCL cells were treated with GB111-NH2 for 1 hour and the cells were evaluated for caspase-3 activation and annexin V staining as indications for apoptosis.

Caspase-3 activation was assessed through the evaluation of caspase-3 enzyme activity (100 ng of protein) with the substrate Ac-DEVD-pNA (N-acetyl-Asp-Glu-Val-Asp-p-nitroaniline) in a colorimetric assay 24 hours post treatment.

### NF-κB Signaling Assay

Cells were transfected by electroporation with NF-κB reporter plasmid. After selection, the cells were harvested, and the luciferase activities were measured and normalized. 5 μM GB111-NH2 inhibited activity of NF-κB reporter in OCI-Ly 19 cells induced by 0.5 μg LPS (24 h induction). 10 μM SC-514, a specific suppressor of NF-κB activity, was used as positive control.

### Cell Apoptosis Assay of Combined Therapy

OCI-Ly19 cells were incubated with 5 μM GB111-NH2 with or without 10 μg Rituximab (anti CD20), Daratumumab (anti CD138) and anti-CD74, cell death was analyzed 24 hours after treatment.

### Inhibition of Purified Recombinant Cathepsins B and S

Recombinant enzymes in buffer (pH 5.5) were treated either at the indicated concentrations of inhibitors or with DMSO (0.1%) for 30 min followed by labeling with GB123 for 30 min. Samples were analyzed by SDS-PAGE and fluorescent signal was measured by scanning of gels with a Typhoon laser flatbed scanner.

### Results

The cathepsins activity in peripheral blood cells and serum of patients with non-Hodgkin's lymphoma (NHL) was investigated. Blood samples were collected with informed consent and institutional ethical committee approval from all patients, at the time of diagnosis, before therapy. Cathepsin B, L and S activity was detected using a fluorescent cathepsin activity-based probe, GB 123, that binds irreversibly to the active protease and labels them fluorescently.

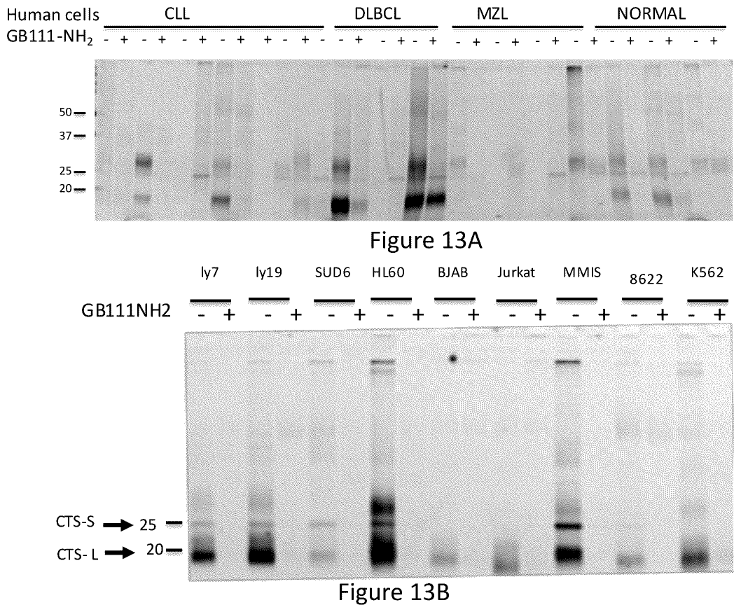

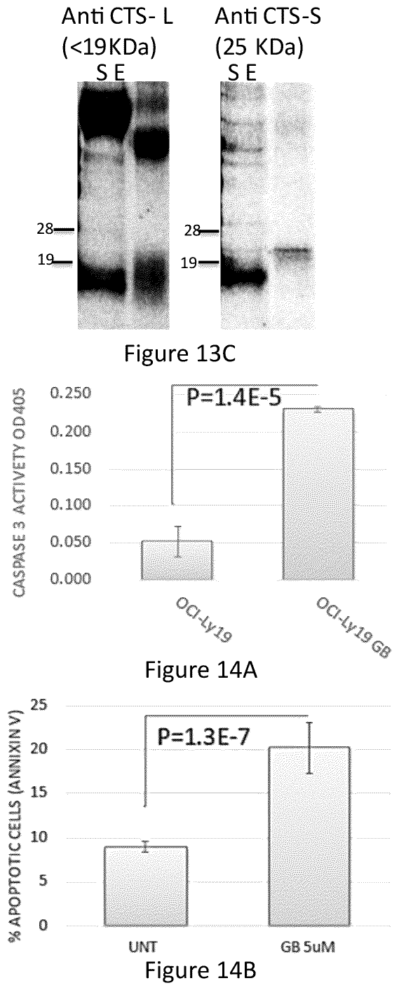

To verify activity dependent labeling, indicated samples were pre-incubated with GB111-NH₂, a potent cathepsin inhibitor, prior to GB123 labeling. After incubation with GB123, a fluorescent scan of SDS-PAGE was done. Cathepsin activity was detected in lysates of cells from human Chronic lymphocytic leukemia (CLL) and Diffuse large B-cell lymphoma (DLBCL) patients as well as healthy population (FIG. 13).

The activity of cathepsins in hematological malignancies cell lines: DLBCL cell lines (OCI-Ly7, OCI-Ly19, SU-DHL-6), Burkitt lymphoma (BJAB), acute myeloid leukemia (HL60), acute T cell leukemia (Jurkat), Multiple myeloma (MM.1s, RPMI 8226) and chronic myeloid leukemia (K562) was evaluated (FIG. 13B). To identify the GB123-labeled bands the labeled lysates were immunoprecipitated with cathepsin S and cathepsin L specific antibodies, the bottom band at ˜20 kDa was found to be cathepsin L and band at ˜25 kDa cathepsin S. The pulled-down protein experiment confirmed the presence of cathepsin S (band at 25 kDa) and L (the band at ˜20 kDa protein ladder) activities (FIG. 13C).

All DLBCL, AML and MM cell lines show cathepsin activity and 25 kDa, corresponding to cathepsins L and S respectively. In BJAB, Jurkat and K562 cells only cathepsin L activity was detected. OCI-Ly7 and OCI-Ly19 showed higher cathepsin L activity than SU-DHL-6. In addition, a weak signal at 32 kDa, corresponding to cathepsin B activity was detected in OCI-Ly7 and OCI-Ly 19 cells, however, the cathepsin B activity is high in HL-60 cells.

As expected, GB111-NH2 abolished cathepsins activity, demonstrating that it is a powerful and potent inhibitor of cathepsins in both patients' cells and in lymphoma cell lines.

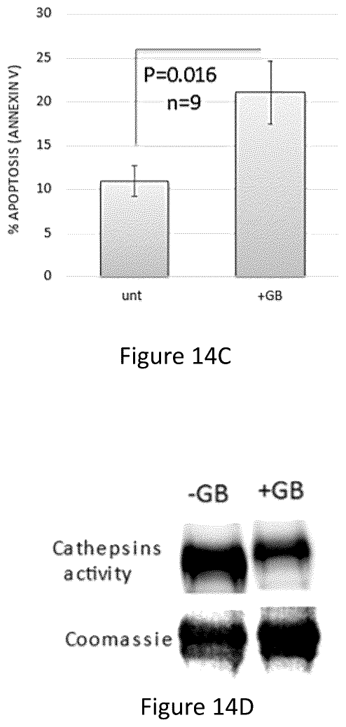

The ability of GB111-NH2 to promote lymphoma cell death was examined. The inhibitor treatment resulted in a significant increase in apoptosis, a 2.5 fold increase in Annexin V staining and a 4.5 fold increase in caspase-3 activity (FIGS. 14A-14B). Furthermore, GB111-NH2 treatment resulted in increased Annexin V staining of blood cells isolated from peripheral blood and bone marrow of CLL patients (FIG. 14C) (p value 0.016). Cathepsin S activity in these cells was initially high and was reduced to 40% (+10%).

In addition, the inhibition effect of cathepsin on NF-κB signaling that is mediated by CD74 cleavage was examined.

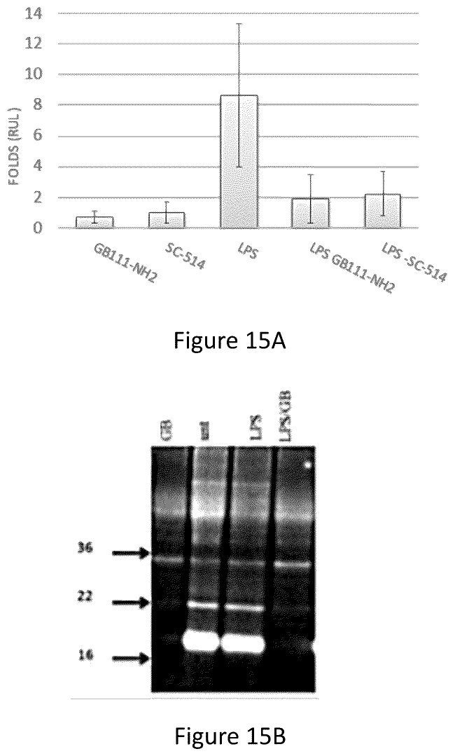

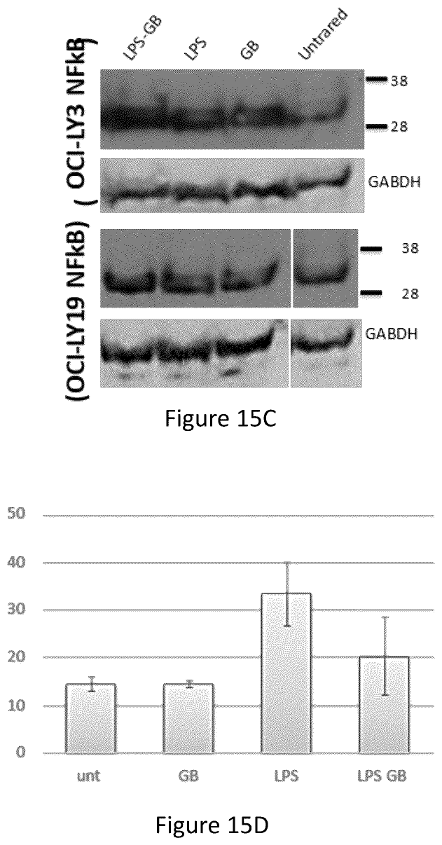

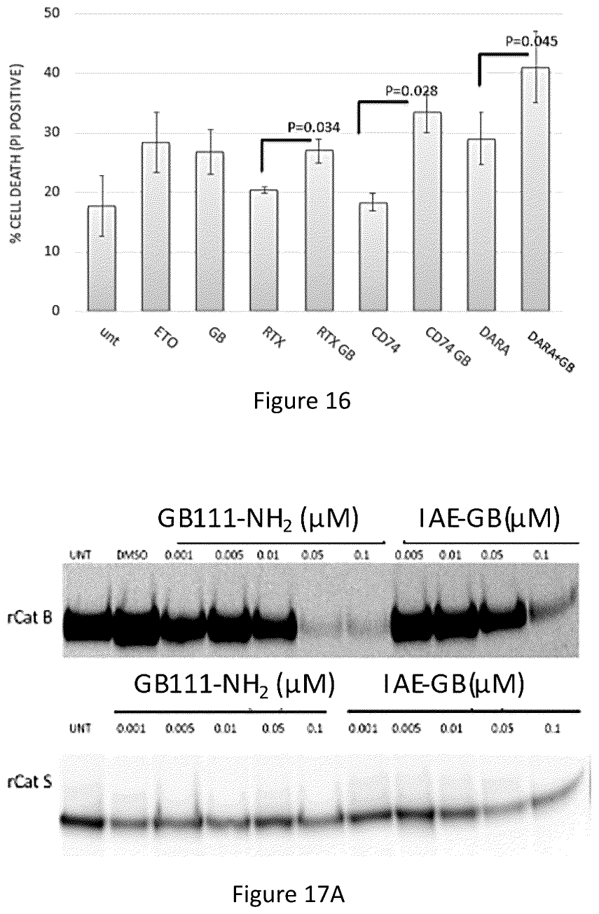

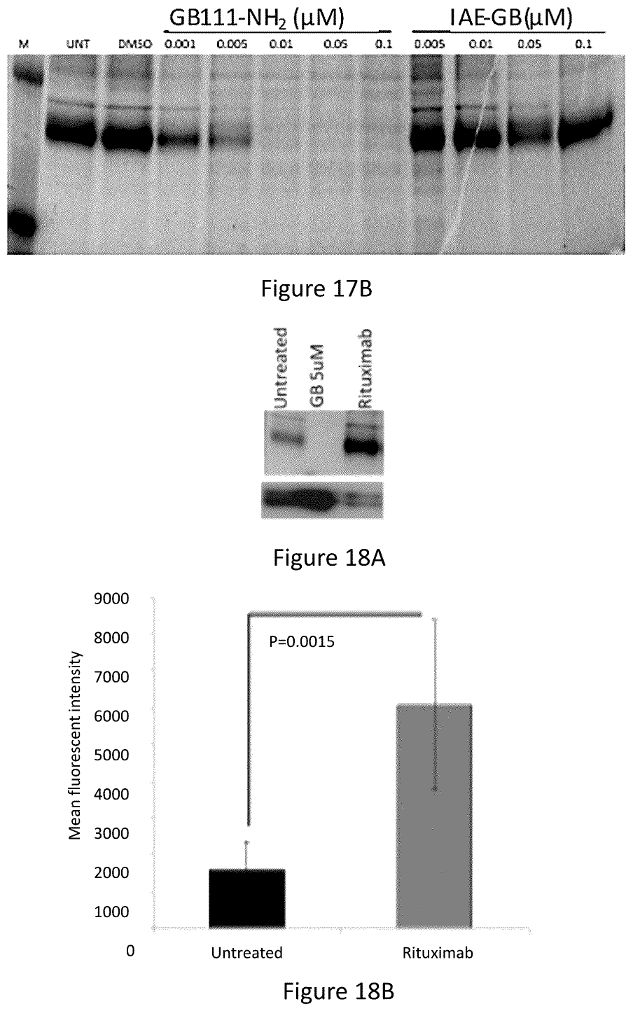

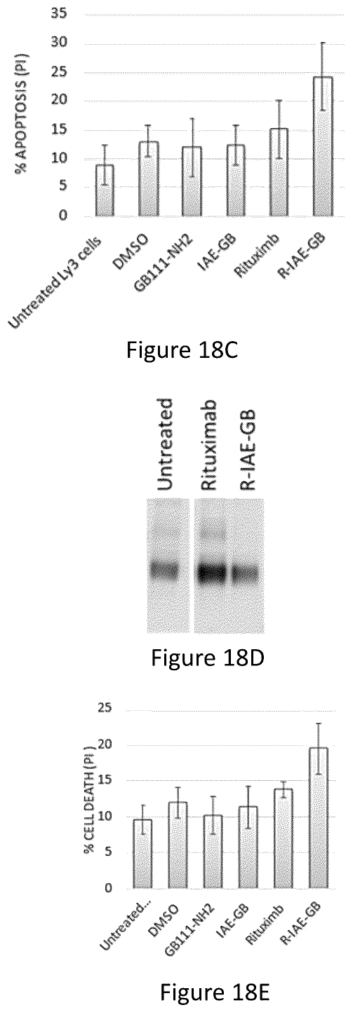

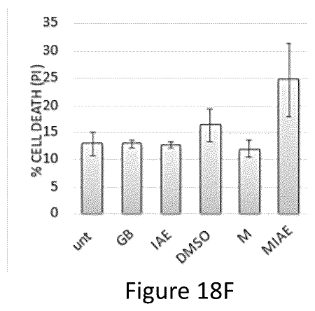

OCI-Ly19 cells stably expressing NF-κB-RE vector (NF-κB activity reporter) were treated with LPS (FIG. 15). As expected, LPS increased the transcriptional activity of NF-κB by 7 folds (FIG. 15A). GB111-NH2 blocked cathepsin activity (FIG. 3B) and suppressed LPS-induced NF-κB transcriptional activity, this inhibition is potent as the specific inhibitor of p65 SC-514, (FIG. 15A).

The CD74 expression was examined by Western blot (WB) and FACS in OCI-Ly3 and OCI-Ly19 treated with GB111-NH2, LPS or LPS pretreated with GB111-NH2. While LPS increased total CD74 expression levels in the cells (FIG. 15C-15D), FACS analysis indicates that GB111-NH2 treatment reduces the surface expression of CD74 (FIG. 15D).

1. A conjugate including any salt thereof and any enantiomer thereof, wherein said conjugate is represented by Formula I: 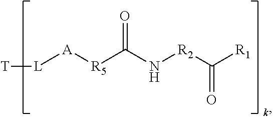
wherein
R1 comprises any one of: chloromethyl ketone, acyloxymethyl ketone, a Michael acceptor, phosphonate, cyano group, or 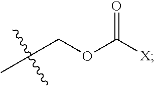
wherein X is selected from a substituted or unsubstituted alkyl; a substituted or unsubstituted alkenyl, a substituted or unsubstituted aryl, a substituted or unsubstituted heteroaryl;
R2 comprises a substituted or unsubstituted alkylamine, or —[C(D′)₂]ₙ—, wherein each D′ is independently H, a substituent, or an optionally substituted C1-C20 alkylamine; and wherein at least one D′ is the optionally substituted C1-C20 alkylamine;
R5 comprises 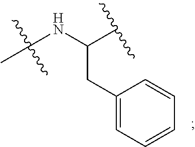 [SMILES: *NC(*)Cc1ccccc1]
A represents at least one amino acid residue;
n is an integer ranging between 1 and 3;
k is an integer ranging between 1 and 30;
L represents a spacer; and
T comprises a small molecule comprising a thiol or an amino and having a binding affinity to an extracellular domain, or a macromolecule comprising a polyamino acid, a glycoprotein, or a polynucleic acid, including any salt, any conjugate or any combination thereof.

2. (canceled)

3. The conjugate of claim 1, wherein A comprises an aromatic amino acid residue, alanine residue, or both.

4. The conjugate of claim 1, wherein said macromolecule is an antibody, or an antigen binding fragment of an antibody; wherein said optionally substituted C1-C20 alkylamine is an optionally substituted C1-C20alkyl-NB′B′ and wherein B′ is R′ or a fluorophore; and wherein said macromolecule is covalently bound to L via a sulfur atom of a cysteine side chain of said macromolecule.

5. (canceled)

6. The conjugate of claim 1, wherein X is

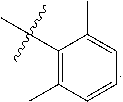 [SMILES: *c1c(C)cccc1C]

7. The conjugate of claim 1, wherein said conjugate is represented by Formula II:

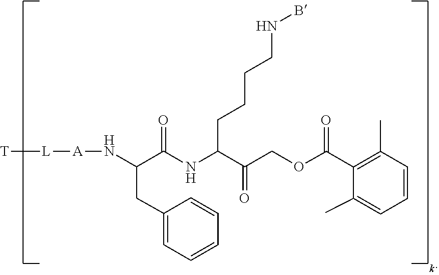

8. The conjugate of claim 1, wherein T and L are covalently bound to each other via a S—C bond; wherein T and L are covalently bound to each other via a click reaction product; and wherein said click reaction product comprises any one of: succinimide-thioether; Michael addition product, azide alkyne cycloaddition product; Diels Alder reaction product; inverse electron demand Diels Alder reaction product; dibenzyl cyclooctyne 1,3-nitrone cycloaddition product, or any combination thereof.

9. (canceled)

10. (canceled)

11. The conjugate of claim 1, represented by Formula III:

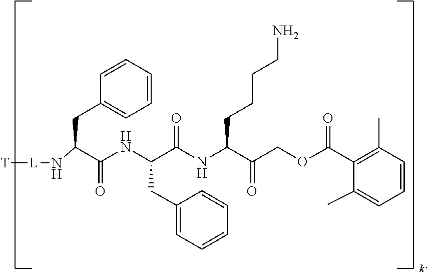

12. The conjugate of claim 1 and wherein said spacer is between 1 and 50 single C—C bonds long and comprises: , wherein a wavy bond represents an attachment point to the macromolecule; wherein c is an integer independently selected from 0 to 10; each L1 independently represents a bond, or is selected from —N—C(═O)—, —C(═O)N—, —S—S—, —S—C(═O), Y—(C0-10)alkyl-X′—(C0-10)alkyl-Y, C1-C10 linear or cyclic alkyl, and a click reaction product, including any combination thereof: wherein each X′ and Y is absent or is independently selected from a heteroatom, an oligomer, a click reaction product, —CONR′—, —CNNR′—, —CSNR′—, —NC(═O)O—, —NC(═S)O—, —NC(═S)N—, —SO2-, —SO—, —SR′, —C(═O)—, —QC(═O)—, —OC(═O)O—, —OC(═S)O—, and —OC(═S)N—; and wherein W represents said click reaction product.

13. (canceled)

14. The conjugate of claim 12, represented by Formula IV:

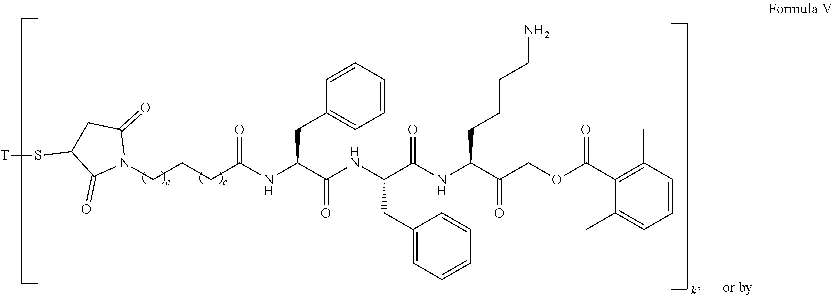

15. The conjugate of claim 14, wherein L1 is a bond.

16. (canceled)

17. (canceled)

18. A compound represented by Formula VI: 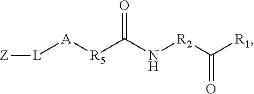
wherein
R1 comprises any one of: chloromethyl ketone, acyloxymethyl ketone, a Michael acceptor, phosphonate, cyano group, or 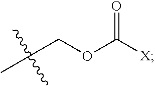
wherein X is selected from a substituted or unsubstituted alkyl; a substituted or unsubstituted alkenyl, a substituted or unsubstituted aryl, a substituted or unsubstituted heteroaryl;
R2 comprises a substituted or unsubstituted alkylamine, or —[C(D′)₂]ₙ-; wherein each D′ is independently H, a substituent, or an optionally substituted C1-C20 alkylamine; and wherein at least one D′ is the optionally substituted C1-C20 alkylamine;
R5 comprises 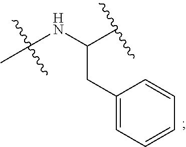 [SMILES: *NC(*)Cc1ccccc1]
A represents at least one amino acid residue;
n is an integer ranging between 1 and 3;
L represents a spacer; and
Z is an electrophilic functionality having a reactivity to a thiol group, an amine group, or both.

19. (canceled)

20. (canceled)

21. (canceled)

22. (canceled)

23. (canceled)

24. (canceled)

25. The compound of claim 18, wherein said compound is

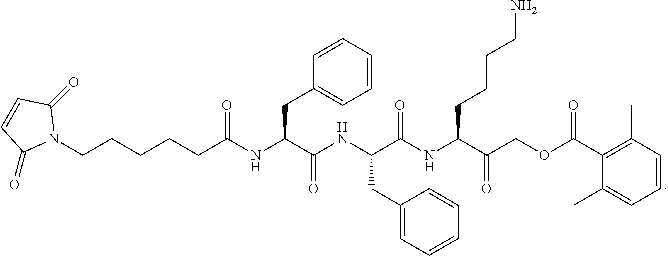 [SMILES: Cc1cccc(C)c1C(=O)OCC(=O)[C@H](CCCCN)NC(=O)[C@H](Cc1ccccc1)NC(=O)[C@H](Cc1ccccc1)NC(=O)CCCCCN1C(=O)C=CC1=O ; "."]

26. A kit comprising the compound of claim 18 and optionally a macromolecule or a small molecule, wherein each of the macromolecule and the small molecule comprises a thio group, an amino group or both.

27. (canceled)

28. (canceled)

29. A pharmaceutical composition comprising the compound of claim 1 including any pharmaceutically acceptable salt thereof, and a pharmaceutically acceptable carrier.

30. (canceled)

31. (canceled)

32. (canceled)

33. (canceled)

34. (canceled)

35. A method for preventing or treating a disease or a disorder associated with a cathepsin activity in a subject in need thereof, comprising administering to said subject a therapeutically effective amount the pharmaceutical composition of claim 29, thereby preventing or treating said disease or said disorder associated with said cathepsin activity in the subject.

36. The method of claim 35, wherein said disease or said disorder is selected from a cell proliferation related disease, an inflammatory, a cardiovascular disease, autoimmune disease, a neurodegenerative disorder, diabetes, obesity, kidney dysfunction, an ocular disease and a viral disease, including any combination thereof.

37. The method of claim 35, wherein said disease or disorder is selected from hypertension, Alzheimer's disease, CLN10 Disease, Gaucher Disease, pancreatitis, Papillion-Lefevre syndrome, periodontitis, Parkinson's Disease, Huntington's Disease, dermatitis, CLN13 Disease, diabetes, pycondysostosis, dementia, cancer and autoimmune arthritis.

38. (canceled)

39. The method of claim 37, further comprising administering at least one immunotherapy treatment to said subject.

40. (canceled)

41. (canceled)

42. (canceled)

43. The method of claim 35, further comprising a step preceding said administering, comprising determining an enzymatic activity of a cathepsin in the subject, wherein an increase of said enzymatic activity in said subject compared to a control, is indicative of said subject being suitable for said treating.

44. The method of claim 43, wherein said determining is via in-vivo imaging or in a sample obtained or derived from the subject; wherein said cathepsin comprises a cysteine cathepsin; optionally wherein said cysteine cathepsin is selected from cathepsin B, cathepsin L and cathepsin S.

45.-84. (canceled)
